(19) United States
(12) Patent Application Publication
(10) Pub. No.: US 2026/0020567 A1
(43) Pub. Date: Jan. 22, 2026

(54) CRYSTALLINE FORMS OF 1,2,4-OXADIAZOLE FUNGICIDES

(71) Applicant: SYNGENTA CROP PROTECTION AG, Basel (CH)

(72) Inventors: Jochen ELSNER, Münchwilen (CH); Miroslav TERINEK, Basel (CH)

(73) Assignee: SYNGENTA CROP PROTECTION AG, Basel (CH)

(21) Appl. No.: 18/997,442

(22) PCT Filed: Jul. 20, 2023
(86) PCT No.: PCT/EP2023/070186

§ 371 Date: Jan. 21, 2025

(30) Foreign Application Priority Data
Jul. 21, 2022 (EP) 22186283.2

(51) Int. Cl. A01N 43/82; A01P 3/00; C07D 271/06

(52) U.S. Cl. CPC A01N 43/82; A01P 3/00; C07D 271/06

(57) ABSTRACT

The invention relates to crystalline forms of compounds of formula (I-A) and (I-B)(I-A)(I-B) compositions comprising said crystalline forms and methods of their use as fungicides.

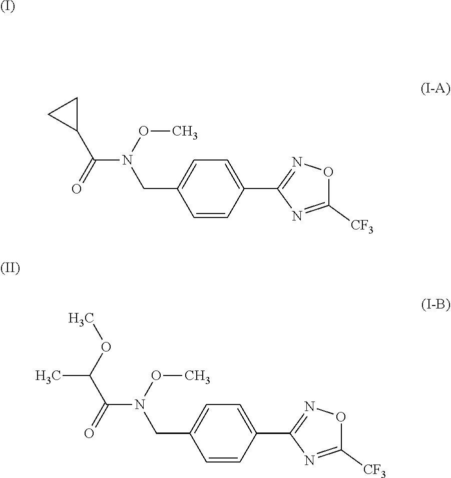 [SMILES: "(I)" ; CON(Cc1ccc(-c2noc(C(F)(F)F)n2)cc1)C(=O)C1CC1 ; "(II)" ; COC(C)C(=O)N(Cc1ccc(-c2noc(C(F)(F)F)n2)cc1)OC]

This invention relates to solid forms of oxadiazole derivatives, compositions comprising the solid forms, to their use in agriculture or horticulture for controlling diseases caused by phytopathogens, especially phytopathogenic fungi, and to methods of controlling diseases on useful plants.

WO2017/055473 discloses that certain oxadiazole derivatives have microbiocidal activity, in particular, fungicidal activity.

In particular, a polymorph of a compound of formula (I-A) and a polymorph of a compound of formula (I-B) is disclosed:

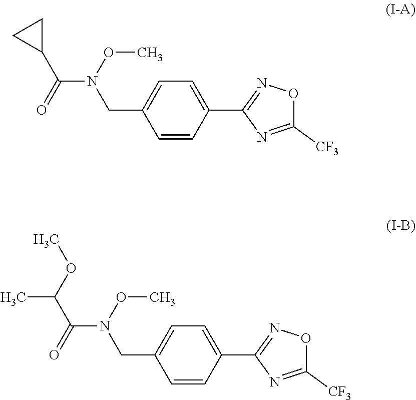 [SMILES: CON(Cc1ccc(-c2noc(C(F)(F)F)n2)cc1)C(=O)C1CC1 ; COC(C)C(=O)N(Cc1ccc(-c2noc(C(F)(F)F)n2)cc1)OC]

Methods for preparing compound of formula (I-A) have been disclosed in WO2018/177894 and in WO2020/212513. Methods for preparing compound of formula (I-B) have been disclosed in WO2018/177894 and in WO2020/212513.

Agrochemical compositions comprising compound (I-A) and (I-B) have been disclosed generically in WO2017/055473. Mixtures of this compound with fungicides are disclosed in WO2018/177894, WO2022117653A1, WO2022106304 and in WO2022117373.

However, the application of certain types of formulations is dependent on the particular form, i.e., polymorphic or amorphous form, used to prepare the formulation. For example, if the form used to prepare a suspension concentrate (SC) is not stable in such a SC formulation, polymorphic conversion might occur in the formulation leading to unwanted crystal growth. Such crystal growth may be detrimental because it may lead to thickening and potentially solidification of the formulation which can in turn lead to blockages in application equipment, e.g., in spray nozzles in agricultural application machinery. Hence, there is a need to provide stable crystalline forms of the above compound to prepare agricultural or pharmaceutical formulations thereof.

New solid forms of this compound and its isomers, their compositions and methods of their preparation and use have now been discovered.

In the context of the present invention, a polymorph is a particular crystal form of a chemical compound that can exist in more than one crystal form in the solid state. A crystal form of a compound contains the constituent molecules arranged in orderly repeating patterns extending in all three spatial dimensions (in contrast, an amorphous solid form has no long-range order in the position of molecules). Different polymorphs of a compound have different arrangements of atoms and or molecules in their crystal structure. When the compound is a biologically active compound, such as an insecticide or fungicide, the difference in crystal structures can lead to different polymorphs having different chemical, physical and biological properties. Properties which may be affected include crystal shape, density, hardness, colour, chemical stability, melting point, hygroscopicity, suspensibility, dissolution rate and biological availability. As such, a specific polymorph may have properties which make it more advantageous in a particular use relative to another polymorph of the same compound: in particular, the physical, chemical and biological properties listed above can have a significant effect on the development of production methods and formulations, the ease with which a compound can be combined in a formulation with other active ingredients and formulation components and the quality and efficacy of plant treatment agents, such as insecticides or fungicides. It is noted that predicting whether the solid state of a compound may be present as more than one polymorph is not possible and nor is it possible to predict the properties of any of these crystal forms.

Several techniques are commonly used to characterize polymorphs. For example, powder X-ray diffraction (pXRD) techniques are often used, other techniques which may be used include differential scanning calorimetry (DSC), thermogravimetric analysis (TGA) and Raman or Infra-red spectroscopy, nuclear magnetic resonance (NMR), gas chromatography, HPLC and in particular single crystal X-ray diffraction.

In a first aspect, the present invention provides a crystalline form of N-methoxy-N-[[4-[5-(trifluoromethyl)-1,2,4-oxadiazol-3-yl]phenyl]methyl]cyclopropanecarboxamide (compound I-A).

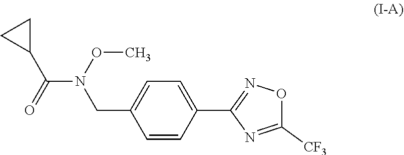 [SMILES: CON(Cc1ccc(-c2noc(C(F)(F)F)n2)cc1)C(=O)C1CC1]

characterized by an X-ray diffraction pattern comprising four or more 2-theta angle values selected from the group 11.9±0.2, 12.5±0.2, 16.5±0.2, 17.1±0.2, 19.5±0.2, 22.1±0.2, 23.9±0.2 and 24.9±0.2 at a temperature of 21-26° C.

In one embodiment of the first aspect, the crystalline form of compound of formula (I-A) is characterized by a powder X-ray diffraction pattern comprising six or more 2-theta angle values selected from the group of 11.9±0.2, 12.5±0.2, 16.5±0.2, 17.1±0.2, 19.5±0.2, 22.1±0.2, 23.9±0.2, 24.9±0.2, 25.1±0.2, and 27.6±0.2 at a temperature of 21-26° C.

In another embodiment of the first aspect, the crystalline form of compound of formula (I-A) is characterized by a powder X-ray diffraction pattern comprising the 2-theta angle values selected from the group of 6.2±0.2, 8.2±0.2, 11.9±0.2, 12.5±0.2, 16.5±0.2, 17.1±0.2, 18.9±0.2, 19.0±0.2, 19.5±0.2, 19.8±0.2, 21.4±0.2, 21.6±0.2, 22.1±0.2, 22.7±0.2, 23.5±0.2, 23.9±0.2, 24.9±0.2, 25.1±0.2, 25.5±0.2, 26.1±0.2, 26.9±0.2, 27.6±0.2, 27.6±0.2, 28.5±0.2 and 29.0±0.2 at a temperature of 21-26° C.

In another embodiment of the first aspect, the crystalline form of compound of formula (I-A) has an X-ray powder diffraction pattern which is substantially the same as the X-ray powder diffraction spectrum shown in FIG. 1 at a temperature of 21-26° C.

In another embodiment of the first aspect, the crystalline form of compound of formula (I-A) is further characterized by the following unit cell parameters at a calculated density [g/cm⁻³] of 1.503:

| | |
|---|---|
| cell-length a | 15.2 ± 0.1 A |
| cell-length b | 4.8 ± 0.1 Å |
| cell-length c | 21.5 ± 0.1 Å |
| cell-angle α | 90 ± 0.2° |
| cell-angle-β | 106.4 ± 0.2° |
| cell-angle γ | 90 ± 0.2° |
| cell-volume | 1508 ± 16 Å³ |

These unit cell parameters have been collected as described under examples. The crystalline form of compound of formula (I-A) is further characterized by a space group of P 2₁/n.

In yet another embodiment of the first aspect, the melting peak of the crystalline form of compound of formula (I-A) is a broad water endotherm in the DSC trace at about 38.6° C. (FIG. 3).

In a second aspect, the present invention provides a crystalline form of N,2-dimethoxy-N-[[4-[5-(trifluoromethyl)-1,2,4-oxadiazol-3-yl]phenyl]methyl]propenamide (compound I-B).

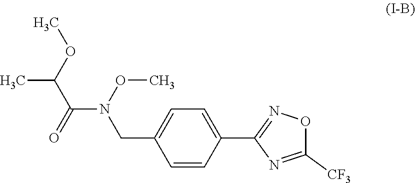 [SMILES: COC(C)C(=O)N(Cc1ccc(-c2noc(C(F)(F)F)n2)cc1)OC]

characterized by an X-ray diffraction pattern comprising four or more 2-theta angle values selected from the group 8.7±0.2, 9.7±0.2, 14.4±0.2, 15.8±0.2, 17.3±0.2 and 20.7±0.2 at a temperature of 21-26° C.

In one embodiment of the second aspect, the crystalline form of compound of formula (I-B) is characterized by a powder X-ray diffraction pattern comprising six or more 2-theta angle values selected from the group 8.7±0.2, 9.7±0.2, 12.1±0.2, 14.4±0.2, 15.8±0.2, 17.3±0.2, 20.7±0.2, 23.0±0.2, 29.1±0.2, and 30.0±0.2 at a temperature of 21-26° C.

In another embodiment of the second aspect, the crystalline form of compound of formula (I-B) is characterized by a powder X-ray diffraction pattern comprising the 2-theta angle values selected from the group of 8.7±0.2, 9.7±0.2, 12.1±0.2, 13.3±0.2, 14.4±0.2, 15.2±0.2, 15.8±0.2, 16.2±0.2, 17.3±0.2, 18.5±0.2, 19.1±0.2, 20.7±0.2, 21.8±0.2, 22.1±0.2, 23.0±0.2, 23.7±0.2, 29.1±0.2, and 30.0±0.2 at a temperature of 21-26° C.

In another embodiment of the second aspect, the crystalline form of compound of formula (I-B) has an X-ray powder diffraction pattern which is substantially the same as the X-ray powder diffraction spectrum shown in FIG. 4 at a temperature of 21-26° C.

In another embodiment of the second aspect, the crystalline form of compound of formula (I-B) is further characterized by the following unit cell parameters at a calculated density [g/cm⁻³] of 1.434:

| | |
|---|---|
| cell length a | 7.4 ± 0.1 Å |
| cell length b | 18.2 ± 0.1 Å |
| cell length c | 12.7 ± 0.1 Å |
| cell angle α | 90 ± 0.2° |
| cell angle β | 104.3 ± 0.2° |
| cell angle γ | 90 ± 0.2° |
| cell volume | 1664 ± 17 Å³ |

These unit cell parameters have been collected as described under examples. The crystalline form of compound of formula (I-B) is further characterized by a space group of P 2₁/n.

In yet another embodiment of the second aspect, the melting peak of the crystalline form of compound of formula (I-B) is a broad water endotherm in the DSC trace at about 63.5° C. (FIG. 6).

According to a third aspect of the invention, there is provided an agrochemical composition comprising a fungicidally effective amount of a crystalline form according to the first aspect of a compound of formula (I-A), or according to the second aspect a compound of formula (I-B) according to the invention. Such an agricultural composition may further comprise at least one additional active ingredient and/or an agrochemical-acceptable diluent or carrier.

In one embodiment of the third aspect there is provided an agrochemical composition comprising a fungicidally effective amount of a crystalline form according to the first aspect of a compound of formula (I-A) according to the invention. Such an agricultural composition may further comprise at least one additional active ingredient and/or an agrochemical-acceptable diluent or carrier.

In another embodiment of the third aspect there is provided an agrochemical composition comprising a fungicidally effective amount of a crystalline form according to the second aspect of a compound of formula (I-B) according to the invention. Such an agricultural composition may further comprise at least one additional active ingredient and/or an agrochemical-acceptable diluent or carrier.

The polymorphs of the invention may be applied in unchanged form but is more preferably incorporated into agrochemical or pharmaceutical compositions, in particular agrochemical compositions, by conventional means.

According to a fourth aspect of the invention, there is provided a method of controlling or preventing infestation of useful plants by phytopathogenic microorganisms, wherein a fungicidally effective amount of a crystalline form according to the first aspect of a compound of formula (I-A), or according to the second aspect a compound of formula (I-B) according to the invention, or a composition comprising the crystalline form according to the first aspect of a compound of formula (I-A) or a compound of formula (I-B), is applied to the plants, to parts thereof or the locus thereof.

In one embodiment of the fourth aspect of the invention, there is provided a method of controlling or preventing infestation of useful plants by phytopathogenic microorganisms, wherein a fungicidally effective amount of a crystalline form according to the first aspect of a compound of formula (I-A) according to the invention, or a composition comprising the crystalline form according to the first aspect of a compound of formula (I-A) is applied to the plants, to parts thereof or the locus thereof.

In another embodiment of the fourth aspect of the invention, there is provided a method of controlling or preventing infestation of useful plants by phytopathogenic microorganisms, wherein a fungicidally effective amount of a crystalline form according to the second aspect of a compound of formula (I-B) according to the invention, or a composition comprising the crystalline form according to the first aspect of a compound of formula (I-B) is applied to the plants, to parts thereof or the locus thereof.

In another embodiment of the invention, there is provided a method of controlling or preventing infestation of genetically modified plants by phytopathogenic microorganisms, especially phytopathogenic fungi, wherein a compound of formula (I-A) or a polymorph of formula (I-A) is applied to said plants. Preferably the genetically modified plants are soybean plants. Examples of genetically modified plants of soybean are, but not limited to, Intacta®, Intacta®2, Intacta® Roundup Ready™ 2 Pro (Intacta® RR2 PRO), Intacta®2 Xtend™, Cultivance, Conkesta Soybean, Conkesta Enlist E3™ Soybean, Enlist™ Soybean, Enlist E3™ Soybean, Roundup Ready™ Soybean, Roundup Ready™ 2 Xtend™, Genuity® Roundup Ready™ 2 Xtend™. Genuity® Roundup Ready 2 Yield™, Herbicide-tolerant Soybean line, Optimum GAT™, Liberty Link™ Soybean, Vistive Gold™, Verdeca HB4 Soybean, Treus™, Plenish™. More preferably the genetically modified plants are Bt soybean plants. Examples of “Bt plants” are for example soybean varieties which are sold under the trade names Intacta®, Intacta®2, Intacta® Roundup Ready™ 2 Pro (Intacta® RR2 PRO), Cultivance, Conkesta Soybean, Conkesta Enlist E3™ Soybean, Enlist™ Soybean, Enlist E3™ Soybean, Roundup Ready™ Soybean, Genuity® Roundup Ready™ 2 Xtend™, Genuity® Roundup Ready 2 Yield™, Herbicide-tolerant Soybean line, Optimum GAT™, Liberty Link™ Soybean, Vistive Gold™, Verdeca HB4 Soybean, Treus™, Plenish™. Even more preferably, the Bt soybean plants are selected from Intacta RR2 PRO®, or Conkesta Enlist E3®.

In another embodiment of the invention, there is provided a method of controlling or preventing infestation of genetically modified plants by phytopathogenic microorganisms, especially phytopathogenic fungi, wherein a compound of formula (I-B) or a polymorph of formula (I-B) is applied to said plants. Preferably the genetically modified plants are soybean plants. Examples of genetically modified plants of soybean are, but not limited to, Intacta®, Intacta®2, Intacta® Roundup Ready™ 2 Pro (Intacta® RR2 PRO), Intacta®2 Xtend™, Cultivance, Conkesta Soybean, Conkesta Enlist E3™ Soybean, Enlist™ Soybean, Enlist E3™ Soybean, Roundup Ready™ Soybean, Roundup Ready™ 2 Xtend™, Genuity® Roundup Ready™ 2 Xtend™, Genuity® Roundup Ready 2 Yield™, Herbicide-tolerant Soybean line, Optimum GAT™, Liberty Link™ Soybean, Vistive Gold™, Verdeca HB4 Soybean, Treus™, Plenish™. More preferably the genetically modified plants are Bt soybean plants. Examples of “Bt plants” are for example soybean varieties which are sold under the trade names Intacta®, Intacta®2, Intacta® Roundup Ready™ 2 Pro (Intacta® RR2 PRO), Cultivance, Conkesta Soybean, Conkesta Enlist E3™ Soybean, Enlist™ Soybean, Enlist E3™ Soybean, Roundup Ready™ Soybean, Genuity® Roundup Ready™ 2 Xtend™, Genuity® Roundup Ready 2 Yield™, Herbicide-tolerant Soybean line, Optimum GAT™, Liberty Link™ Soybean, Vistive Gold™, Verdeca HB4 Soybean, Treus™, Plenish™. Even more preferably, the Bt soybean plants are selected from Intacta RR2 PRO®, or Conkesta Enlist E3®.

In a preferred embodiment of the invention, there is provided a method for controlling pests on soybean plants, wherein said soybean plants are Bt soybean plants, preferably Bt soybean plants selected from Intacta RR2 PRO®, or Conkesta Enlist E3®, characterized by comprising the step of contacting the plant, parts thereof, propagation material thereof, the pests, their food source, habitat, or breeding ground with a compound of formula (I-A)

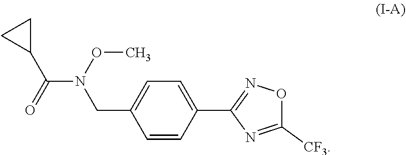 [SMILES: CON(Cc1ccc(-c2noc(C(F)(F)F)n2)cc1)C(=O)C1CC1]

In another preferred embodiment of the invention, there is provided a method for controlling pests on soybean plants, wherein said soybean plants are Bt soybean plants, preferably Bt soybean plants selected from Intacta RR2 PRO®, or Conkesta Enlist E3®, characterized by comprising the step of contacting the plant, parts thereof, propagation material thereof, the pests, their food source, habitat, or breeding ground with a polymorph of a compound of formula (I-A) according to the present invention.

In a preferred embodiment of the invention, there is provided a method for controlling pests on soybean plants, wherein said soybean plants are Bt soybean plants, preferably Bt soybean plants selected from Intacta RR2 PRO®, or Conkesta Enlist E3®, characterized by comprising the step of contacting the plant, parts thereof, propagation material thereof, the pests, their food source, habitat, or breeding ground with a compound of formula (I-B).

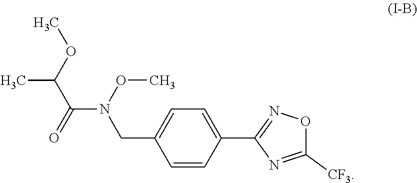 [SMILES: COC(C)C(=O)N(Cc1ccc(-c2noc(C(F)(F)F)n2)cc1)OC]

In another preferred embodiment of the invention, there is provided a method for controlling pests on soybean plants, wherein said soybean plants are Bt soybean plants, characterized by comprising the step of contacting the plant, parts thereof, propagation material thereof, the pests, their food source, habitat, or breeding ground with a polymorph of a compound of formula (I-B) according to the present invention.

Commercially available examples of genetically modified soybean plants, which can preferably be treated according to the invention, include commercially available products such as plant seeds, which are under the Intacta®, Intacta®2, Intacta®2 Xtend™, Intacta® Roundup Ready™ 2 Pro (Intacta® RR2 PRO), Cultivance, Conkesta Soybean, Conkesta Enlist E3™ Soybean, Enlist™ Soybean, Enlist E3™ Soybean, Roundup Ready™ Soybean, Roundup Ready™ 2 Xtend™, Genuity® Roundup Ready™ 2 Xtend™ Genuity® Roundup Ready 2 Yield™, Herbicide-tolerant Soybean line, Optimum GAT™, Liberty Link™ Soybean, Vistive Gold™, Verdeca HB4 Soybean, Treus™, or Plenish™ trade names are sold or distributed.

Commercially available examples of Bt soybean plants, which can preferably be treated according to the invention, include commercially available products such as plant seeds, which are sold under the trade names Intacta®, Intacta®2, Intacta®2 Xtend™, Intacta® Roundup Ready™ 2 Pro (Intacta® RR2 PRO), Cultivance, Conkesta Soybean, Conkesta Enlist E3™ Soybean, Enlist™ Soybean, Enlist E3™ Soybean, Roundup Ready™ Soybean, Roundup Ready™ 2 Xtend™, Genuity® Roundup Ready™ 2 Xtend™ Genuity® Roundup Ready 2 Yield™, Herbicide-tolerant Soybean line, Optimum GAT™, Liberty Link™ Soybean, Vistive Gold™, Verdeca HB4 Soybean, Treus™, or Plenish™.

Preferably genetically modified soybean plants, which can be treated according to the invention, are selected from Intacta RR2 PRO®, or Conkesta Enlist E3®.

According to a fifth aspect of the invention, there is provided the use of a crystalline form according to the first aspect of a compound of formula (I-A), or according to the second aspect of a compound of formula (I-B) according to the invention as a fungicide. According to this aspect of the invention, the use may exclude methods for the treatment of the human or animal body by surgery or therapy.

In one embodiment of the fifth aspect of the invention, there is provided the use of a crystalline form according to the first aspect of a compound of formula (I-A) according to the invention as a fungicide. According to this aspect of the invention, the use may exclude methods for the treatment of the human or animal body by surgery or therapy.

In another embodiment of the fifth aspect of the invention, there is provided the use of a crystalline form according to the second aspect of a compound of formula (I-B) according to the invention as a fungicide. According to this aspect of the invention, the use may exclude methods for the treatment of the human or animal body by surgery or therapy.

In another embodiment of the invention, there is provided the use of a compound of formula (I-A) or the polymorph thereof (I-A) for controlling phytopathogenic fungi in genetically modified plants. Preferably the genetically modified plants are soybean plants. More preferably said genetically modified soybean plants are Bt soybean plants, even more preferably Bt soybean plants selected from Intacta RR2 PRO®, or Conkesta Enlist E3®.

In another embodiment of the invention, there is provided the use of a compound of formula (I-B) or the polymorph thereof (I-B) for controlling phytopathogenic fungi in genetically modified plants. Preferably said genetically modified plants are soybean plants. More preferably said genetically modified soybean plants are Bt soybean plants, even more preferably Bt soybean plants selected from Intacta RR2 PRO®, or Conkesta Enlist E3®.

In another embodiment of the invention, there is provided the use of a compound of formula (I-A) or the polymorph thereof (I-A) for controlling Phakopsora pachyrhizi in genetically modified plants. Preferably the genetically modified plants are soybean plants. More preferably said genetically modified soybean plants are Bt soybean plants, even more preferably Bt soybean plants selected from Intacta RR2 PRO®, or Conkesta Enlist E3®.

In another embodiment of the invention, there is provided the use of a compound of formula (I-B) or the polymorph thereof (I-B) for controlling Phakopsora pachyrhizi in genetically modified plants. Preferably the genetically modified plants are soybean plants. More preferably said genetically modified soybean plants are Bt soybean plants, even more preferably Bt soybean plants selected from Intacta RR2 PRO®, or Conkesta Enlist E3®.

In a preferred embodiment of the invention, there is provided the use of a compound of formula (I-A)

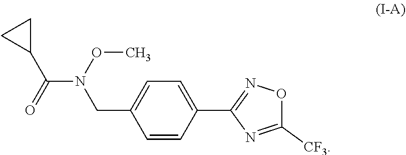 [SMILES: CON(Cc1ccc(-c2noc(C(F)(F)F)n2)cc1)C(=O)C1CC1]

for controlling phytopathogenic fungi in genetically modified soybean plants. Preferably said genetically modified soybean plants are Bt soybean plants, preferably Bt soybean plants selected from Intacta RR2 PRO®, or Conkesta Enlist E3®.

In a preferred embodiment of the invention, there is provided the use of a compound of formula (I-A)

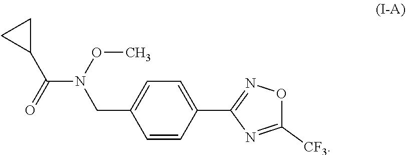 [SMILES: CON(Cc1ccc(-c2noc(C(F)(F)F)n2)cc1)C(=O)C1CC1]

for controlling Phakopsora pachyrhizi in genetically modified soybean plants. Preferably said genetically modified soybean plants are Bt soybean plants, preferably Bt soybean plants selected from Intacta RR2 PRO®, or Conkesta Enlist E3®.

In another preferred embodiment of the invention, there is provided the use of a polymorph of compound of formula (I-A) according to the present invention, for controlling Phakopsora pachyrhizi in genetically modified soybean plants. Preferably said genetically modified soybean plants are Bt soybean plants, preferably Bt soybean plants selected from Intacta RR2 PRO®, or Conkesta Enlist E3®.

In a preferred embodiment of the invention, there is provided the use of a compound of formula (I-B)

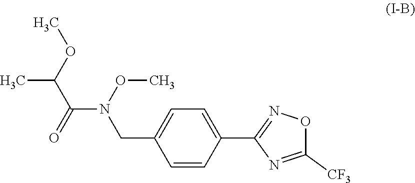 [SMILES: COC(C)C(=O)N(Cc1ccc(-c2noc(C(F)(F)F)n2)cc1)OC]

for controlling phytopathogenic fungi in genetically modified soybean plants. Preferably said genetically modified soybean plants are Bt soybean plants, preferably Bt soybean plants selected from Intacta RR2 PRO®, or Conkesta Enlist E3®.

In a preferred embodiment of the invention, there is provided the use of a compound of formula (I-B)

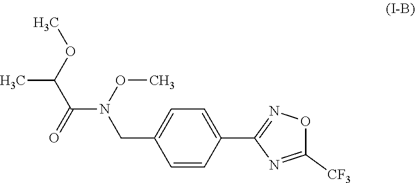 [SMILES: COC(C)C(=O)N(Cc1ccc(-c2noc(C(F)(F)F)n2)cc1)OC]

for controlling Phakopsora pachyrhizi in genetically modified soybean plants. Preferably said genetically modified soybean plants are Bt soybean plants, preferably Bt soybean plants selected from Intacta RR2 PRO®, or Conkesta Enlist E3®.

In another preferred embodiment of the invention, there is provided the use of a polymorph of compound of formula (I-B) according to the present invention, for controlling Phakopsora pachyrhizi in genetically modified soybean plants. Preferably said genetically modified soybean plants are Bt soybean plants, preferably Bt soybean plants selected from Intacta RR2 PRO®, or Conkesta Enlist E3®.

Commercially available examples of genetically modified soybean plants, which can preferably be treated according to the invention, include commercially available products such as plant seeds, which are under the Roundup Ready® (RR1), Roundup Ready 2 Xtend®, Roundup Ready 2 Yield®, XtendFlex®, Intacta RR2 PRO®, Intacta 2 Xtend®, Vistive® Gold™, Conkesta Enlist E3®, Conkesta E3®, Enlist E3®, Genuity® Roundup Ready 2 Yield™, Genuity® Roundup Ready™ 2 Xtend™, Herbicide-tolerant Soybean line, Optimum GAT™, Liberty Link™ Soybean, Vistive Gold™, Verdeca HB4 Soybean, Treus™, or Plenish™ trade names sold or distributed.

Preferably genetically modified soybean plants, which can be treated according to the invention, are selected from Intacta RR2 PRO®, or Conkesta Enlist E3®.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain, and timber); and those pests associated with the damage of man-made structures. The term pest encompasses all stages in the life cycle of the pest.

As used herein, the term “crops” is to be understood as including also crop plants which have been so transformed using recombinant DNA techniques that they are capable of synthesising one or more selectively acting toxins, such as are known, for example, from toxin-producing bacteria, especially those of the genus Bacillus.

As used herein, the term “genetically modified plant” or “genetically modified soybean plant” refers to a plant or soybean plant, in which the genetic material has been altered in a way that does not occur naturally by mating and/or natural recombination. These plants are also called transgenic or genetically engineered plants. Genetic modification of plants involves adding a specific stretch of DNA into the plant's genome, giving it new or different characteristics. This could include changing the way the plant grows or making it resistant to a particular disease. Examples of genetically modified soybean plants are available under the tradenames YIELD GARD®, Intacta®, Intacta®2, Intacta® Roundup Ready™ 2 Pro (Intacta® RR2 PRO), Cultivance, Conkesta Soybean, Conkesta Enlist E3™ Soybean, Enlist™ Soybean, Enlist E3™ Soybean, Roundup Ready™ Soybean, Genuity® Roundup Ready™ 2 Xtend™, Genuity® Roundup Ready 2 Yield™ Herbicide-tolerant Soybean line, Optimum GAT™, Liberty Link™ Soybean, Vistive Gold™, Verdeca HB4 Soybean, Treus™, Plenish™.

As used herein, the term “Bt soybean plant” refers to soybean plants that are genetically engineered soybeans that produce an insecticidal protein like the one naturally produced by the bacteria species Bacillus thuringiensis, for example by the genes CryIA(a), CryIA(b), CryIA(c), CryIIA, CryIIIA, CryIIIB2, Cry9c, Cry2Ab, Cry3Bb and CryIF and also combinations thereof. These soybeans that are genetically engineered to produce the same toxin as Bacillus thuringiensis (Bt) in every cell of the plant, with the goal of protecting the soybean from pests, are referred to herein as “Bt soybeans”. Examples Bt soybean plants are Intacta RR2 PRO®, or Conkesta Enlist E3®.

As used herein, the term “effective amount” refers to the amount of the compound, or a salt thereof, which, upon single or multiple applications provides the desired effect.

As used herein, the term “room temperature” or “RT” or “rt” or “ambient temperature” refer to a temperature of about 15° C. to about 35° C. For example, rt can refer to a temperature of about 20° C. to about 30° C.

An effective amount is readily determined by the skilled person in the art, using known techniques and by observing results obtained under analogous circumstances. In determining the effective amount, several factors are considered including, but not limited to the type of plant or derived product to be applied; the pest to be controlled & its lifecycle; the particular compound applied; the type of application; and other relevant circumstances.

The compounds of formula (I-A) and (I-B) according to the present invention can be made as shown in the following Scheme (Scheme 1)

The compound of formula (I-A) and (I-B) is prepared according to the process of the invention, said process comprising the step of reacting a compound of formula (II-A) or (II-B) with a compound of Formula (III) or a compound of formula (IV). This reaction is shown in Scheme 1.

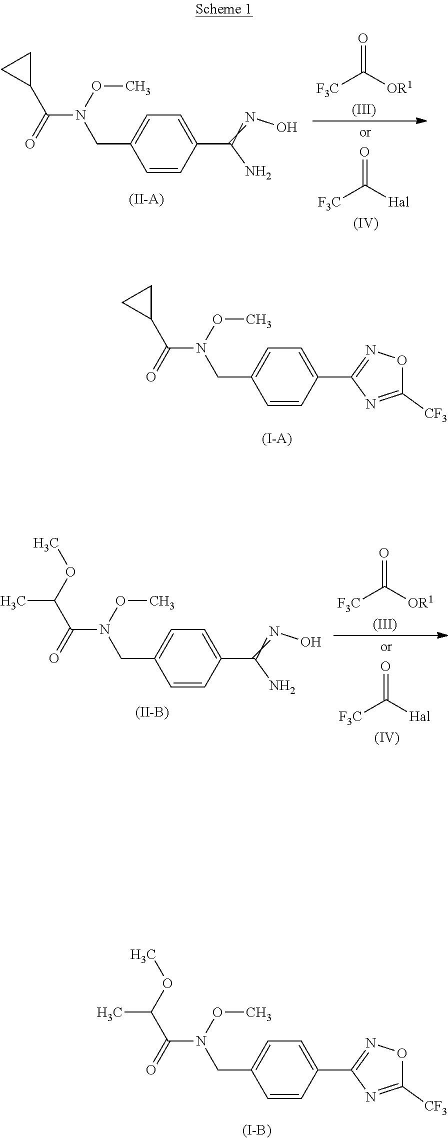

For the purpose of the present invention, the compound of formula (II) is used in the process of the invention in any of its tautomeric forms with different (E)/(Z)-configurations.

Examples of compounds of formula (III) suitable for use in the process of the invention include those wherein R¹ is selected from methyl, ethyl, n-propyl, iso-propyl, n-butyl, sec-butyl, tert-butyl, and iso-butyl. Preferably, R¹ in the compound of formula (III) is methyl or ethyl. The compound of formula (III) is more preferably methyl 2,2,2-trifluoroacetate or ethyl 2,2,2-trifluoroacetate.

Alternatively, examples of compounds of formula (III) suitable for use in the process of the invention include those wherein R¹ is selected from hydrogen or alkyl-C(═O)-alkyl. Examples for examples of compounds of formula (III) suitable for use in the process of the invention include trifluoroacetic acid, trifluoroacetic ester, trifluoroacetic anhydride.

Examples of compounds of formula (IV) suitable for use in the process of the invention include those wherein Halogen is selected from chloride, bromide, or fluoride. The compound of formula (IV) is preferably trifluoroacetyl chloride.

Alternatively, compounds of formula (I-A) or (I-B) can be prepared from compounds of formula (II-A) or (II-B) via reactions with trifluoroacetic acid, trifluoroacetic ester, trifluoroacetic anhydride, or trifluoroacetyl halide (including trifluoroacetyl fluoride, trifluoroacetyl chloride, and trifluoroacetyl bromide), optionally in the presence of a base (e.g., pyridine or 4-dimethylaminopyridine) in a suitable solvent, (e.g., toluene, ethyl acetate, tetrahydrofuran, 2-methyl tetrahydrofuran, or ethanol), at temperatures between 0° C. and 75° C. For related examples, see WO 2003/028729, WO 2017/055473, and WO 2010/045251.

In an embodiment of the process of the invention, the compound of formula (I-A) or (I-B) is advantageously prepared from a compound of formula (II) (II-A) or (II-B)) via reaction with an amount of from 1.0 to 2.0 equivalents of a compound of formula (III).

In a preferred embodiment of the invention, the process of the invention is carried out in the presence of at least one base.

According to one embodiment, the process of the invention is carried out in the presence of at least one base and, optionally, at least one solvent.

Examples of suitable bases include inorganic bases and organic bases. Preferred bases are selected from the group consisting of tertiary amines, substituted or non-substituted pyridine, bicyclic amines and mixtures thereof, NaH, alkali hydroxides, alkali metal C₁₋₆ alkoxylates and alkaline earth metal C₁₋₆ alkoxylates such as, for instance, sodium methoxide, sodium hydroxide, sodium ethoxide, sodium tert-butoxide, potassium tert-butoxide, potassium pentoxide, potassium carbonate (K₂CO₃), sodium carbonate (Na₂CO₃), sodium hydrogen carbonate (NaHCO₃), triethylamine and 3,5-lutidine or 2.6-lutidine.

As used herein, the term “alkali metal” refers to the elements in group 1 of the Periodic Table, preferably to lithium (Li), sodium (Na), or potassium (K).

As used herein, the term “alkaline earth metal” refers to the elements in group 2 of the Periodic Table, preferably to magnesium (Mg), or calcium (Ca).

Examples of suitable solvents include, for instance, aromatic solvents such as toluene, xylenes and dichlorobenzene or polar solvents such as tetrahydrofuran (THF) and dimethyl carbonate (DMC) or non-polar solvents such as methylcyclohexane (MCH).

Further examples of solvents include, but not limited to methanol, ethanol, tert-butanol, 2-methyltetrahydrofuran, N,N-dimethylformamide, dimethylsulfoxide, pyridine, pyrrolidine, N-methyl-2-pyrrolidone, toluene, and dioxane.

The process of the invention is usually carried out at a temperature from 0° C. to 60° C., preferably from 0° C. to 40° C., more preferably from 0° C. to 25° C., or more preferably between 25 to 40° C. Very good results have been obtained when the process of the invention is carried out at a temperature from 0° C. to 25° C. or between 25 to 40° C.

The process of the invention is usually carried out at a pressure from 1 atm to 5 atm (a standard atmosphere, abbreviated atm, is the unit of pressure equal to the average atmospheric pressure at sea level).

Typical reaction times are usually in the range from 1 to 16 hours. Very good results have been obtained when the process of the invention is carried out at reaction times of 1 to 2 hours.

The process disclosed in Scheme 1 typically further comprises the step of isolating the compound of formula (I-A) or (I-B) using an aqueous medium. The compound of formula (I-A) or (I-B) is advantageously isolated from the reaction mixture using an aqueous medium, typically by an extractive work-up.

In a preferred embodiment of the process of the invention, the aqueous medium is an aqueous acidic medium. The aqueous acidic medium is generally prepared by addition of one or more acids to the aqueous medium. Preferably, the process of the invention further comprises the step of isolating the compound of formula (I-A) or (I-B) using an aqueous acidic medium.

It has been found that an aqueous acidic medium during reaction work-up may provide the most suitable medium for purification of compounds of formula (I-A) or (I-B) In particular, it has been surprisingly found that an improvement in the isolated yield can be obtained by adding an aqueous acidic medium to the reaction mixture.

The aqueous acidic medium typically comprises an acid selected from the group consisting of acetic acid, citric acid, sulfuric acid, hydrochloric acid (HCl), HCl/water, HCl/dioxane. Preferably, the aqueous acidic medium comprises citric acid.

The aqueous acidic medium preferably has a pH of from 2.0 to 6.0, more preferably of from 4.9

Methods for preparing compound of formula (II-A) has been disclosed in WO2018/177894 and in WO2020/212513. Methods for preparing compound of formula (II-B) has been disclosed in WO2018/177894 and in WO2020/212513.

Methods for preparing compound of formula (I-A) has been disclosed in WO2018/177894 and in WO2020/212513. Methods for preparing compound of formula (I-B) has been disclosed in WO2018/177894 and in WO2020/212513.

The oil obtained for compound of formula (I-A) was dissolved in a suitable solvent, for instance a non-polar solvent such as methylcyclohexane, the solution was allowed to cool until crystal growth occurred. The obtained crystals are filtered, washed with a suitable solvent, for instance a non-polar solvent such as, but not limited to methylcyclohexane, to obtain a white crystalline product.

It has been found that use of a non-polar solvent for crystallisation of compounds of formula (I-A) or (I-B) was advantageous.

Suitable solvents are, in particular, aromatic and aliphatic hydrocarbons which may or may not be substituted (with substantially non-polar groups), the choice thereof being determined chiefly by economic considerations. Examples of suitable non-polar solvents are benzene, toluene, n-pentane, n-hexane, n-octane, methylcyclohexane or cyclohexane.

In particular, it has been surprisingly found that a crystalline form of compound of formula (I-A) can be obtained by the above procedure. The crystalline form of compound (I-A) results in advantages over the oil obtained using the procedure disclosed in WO2018/177894 and in WO2020/212513 like improved work-up (filterability, purity) resulting in improved biological activity.

The oil or solid form obtained for compound of formula (I-B) was dissolved in a suitable solvent, for instance a non-polar solvent such as, but not limited to methylcyclohexane, the solution was allowed to cool until crystal growth occurred. The obtained crystals are filtered, washed with a suitable solvent, for instance a non-polar solvent such as methylcyclohexane, to obtain a white crystalline product.

Single-crystal X-ray diffraction analysis confirmed the presence of the polymorph of the invention for compound of formula (I-A) and compound of formula (I-B).

The agrochemical compositions comprising the polymorphic form or compound of formula (I-A), or compound of formula (I-B) have a very advantageous spectrum of activities for protecting animals and useful plants against attack and damage by fungi, in particular protecting useful plants against attack and damage by fungi.

In particular, use of a specific polymorph of compound (I-A) or compound (I-B) of the present invention may allow use of new formulations compared with existing polymorphic/amorphous/oily forms of a compound. This might be advantageous for a number of reasons. For example, a suspension concentrate (SC) formulation may be preferred over an emulsion concentrate (EC) because the lack of solvent in the SC often means that the formulation is likely to be less phytotoxic than an equivalent EC formulation—however, if the existing form of a compound is not stable in such an SC formulation, polymorphic conversion might occur leading to unwanted crystal growth. Such crystal growth is detrimental because it leads to, for example, thickening and potentially solidification of the formulation which can lead to blockages in application equipment, e.g., in spray nozzles in agricultural application machinery. Using a stable polymorphic form would overcome these issues.

A further particular advantageous use of a specific polymorph of compound (I-A) or compound (I-B) of the present invention may lead to improved purity of the final product by reducing the amount and number of impurities within the specific polymorph of compound (I-A) or compound (I-B) of the present invention.

Assaying the solid phase for the presence of crystals may be carried out by conventional methods known in the art. For example, it is convenient and routine to use powder X-ray diffraction techniques. Other techniques which may be used include differential scanning calorimetry (DSC), thermogravimetric analysis (TGA) and Raman or Infra-red spectroscopy, NMR, gas chromatography or HPLC. Single crystal X-ray diffraction is especially useful in identifying crystal structures.

The polymorphs of the invention may be applied in unchanged form but are more preferably incorporated into agrochemical compositions by conventional means. Accordingly, in a further aspect, the invention provides an agrochemical composition comprising a polymorph of the invention as defined above and at least one agriculturally acceptable carrier or diluent.

In addition, compositions of the invention may comprise a polymorph of the invention according to the first or second aspect. In one embodiment compositions of the invention may comprise a polymorph of the invention according to the first aspect. In another embodiment compositions of the invention may comprise a polymorph of the invention according to the second aspect.

The agrochemical compositions comprising the polymorph of the present invention are preventively and/or curatively valuable active ingredients in the field of pest control, even at low rates of application, have a favourable biocidal spectrum and are well tolerated by warm-blooded species, fish, and plants. Compositions of the invention may act against all or only individual developmental stages of normally sensitive, but also resistant, animal pests, such as insects or representatives of the order Acarina. The insecticidal or acaricidal activity of the compositions can manifest itself directly, e.g., in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

As such, the agrochemical compositions comprising the polymorph or polymorphs of the present invention can be used for the control of plant pathogenic insects on a number of plant species. Accordingly, the invention also provides a method of preventing or controlling insect infection on plants or plant propagation material comprising treating the plant or plant propagation material with an insecticidally effective amount of an agricultural composition of the invention.

Diastereomeric mixtures or racemic mixtures of compounds of formula (I-A) or (I-B), in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.

Enantiomeric mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

As an example, compounds with more than one asymmetric carbon atoms may exist in diastereomeric forms which can be optionally separated using for example supercritical fluid chromatography (SFC) chromatography with chiral columns. Such diastereomers can show a different fungicidal activity profile, but all isomers and diastereomers form part of this invention The polymorphs of compounds of formula (I-A) or (I-B) and, where appropriate, the tautomers thereof, in each case in free form or in salt form, can, if appropriate, also be obtained in the form of hydrates and/or include other solvents, for example those which may have been used for the crystallization of compounds which are present in solid form.

The polymorphs of compounds of formula (I-A) and (I-B) of the present invention have, for practical purposes, a very advantageous level of biological activity for protecting plants against diseases that are caused by fungi.

The polymorphs of compounds of formula (I-A) and (I-B) according to the invention can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The polymorphs of compounds of formula (I-A) and (I-B) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a polymorph of compound of formula (I-A) or (I-B) according to the invention is applied to the plants, to parts thereof or the locus thereof.

In one embodiment, the present invention relates to a method for controlling or preventing infestation of plants or plant propagation material and/or harvested food crops susceptible to microbial attack by treating plants or plant propagation material and/or harvested food crops wherein an effective amount a polymorph of compound of formula (I-A) according to the first aspect of the invention is applied to the plants, to parts thereof or the locus thereof.

In another embodiment, the present invention relates to a method for controlling or preventing infestation of plants or plant propagation material and/or harvested food crops susceptible to microbial attack by treating plants or plant propagation material and/or harvested food crops wherein an effective amount a polymorph of compound of formula (I-B) according to the second aspect of the invention is applied to the plants, to parts thereof or the locus thereof.

It is also possible to use a polymorph of compound of formula (I-A) or (I-B) according to the invention as a fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

The polymorph of compound of formula (I-A) or (I-B), according to the invention, can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The polymorph of compound of formula (I-A) or (I-B) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

Furthermore, the polymorph of compound of formula (I-A) or (I-B) according to the invention can be used for controlling fungi in related areas, for example in the protection of technical materials, including wood and wood related technical products, in food storage, in hygiene management.

In addition, the invention could be used to protect non-living materials from fungal attack, e.g., lumber, wall boards and paint.

The polymorph of compound of formula (I-A) or (I-B) according to the invention are for example, effective against fungi and fungal vectors of disease as well as phytopathogenic bacteria and viruses.

The polymorphs of the invention may be used to control plant diseases caused by a broad spectrum of fungal plant pathogens in the Basidiomycete, Ascomycete, Oomycete and/or Deuteromycete, Blasocladiomycete, Chrytidiomycete, Glomeromycete and/or Mucoromycete classes.

The composition is effective in controlling a broad spectrum of plant diseases, such as foliar pathogens of ornamental, turf, vegetable, field, cereal, and fruit crops.

These pathogens may include: Oomycetes, including Phytophthora diseases such as those caused by Phytophthora capsici, Phytophthora infestans, Phytophthora sojae, Phytophthora fragariae, Phytophthora nicotianae, Phytophthora cinnamomi, Phytophthora citricola, Phytophthora citrophthora and Phytophthora erythroseptica; Pythium diseases such as those caused by Pythium aphanidermatum, Pythium arrhenomanes, Pythium graminicola, Pythium irregulare and Pythium ultimum; diseases caused by Peronosporales such as Peronospora destructor, Peronospora parasitica, Plasmopara viticola, Plasmopara halstedii, Pseudoperonospora cubensis, Albugo candida, Sclerophthora macrospora and Bremia lactucae; and others such as Aphanomyces cochlioides, Labyrinthula zosterae, Peronosclerospora sorghi and Sclerospora graminicola;

Ascomycetes, including blotch, spot, blast or blight diseases and/or rots for example those caused by Pleosporales such as Stemphylium solani, Stagonospora tainanensis, Spilocaea oleaginea, Setosphaeria turcica, Pyrenochaeta lycoperisici, Pleospora herbarum, Phoma destructiva, Phaeosphaeria herpotrichoides, Phaeocryptocus gaeumannii, Ophiosphaerella graminicola, Ophiobolus graminis, Leptosphaeria maculans, Hendersonia creberrima, Helminthosporium triticirepentis, Setosphaeria turcica, Drechslera glycines, Didymella bryoniae, Cycloconium oleagineum, Corynespora cassiicola, Cochliobolus sativus, Bipolaris cactivora, Venturia inaequalis, Pyrenophora teres, Pyrenophora tritici-repentis, Alternaria alternata, Alternaria brassicicola, Alternaria solani and Alternaria tomatophila, Capnodiales such as Septoria tritici, Septoria nodorum, Septoria glycines, Cercospora arachidicola, Cercospora sojina, Cercospora zeae-maydis, Cercosporella capsellae and Cercosporella herpotrichoides, Cladosporium carpophilum, Cladosporium effusum, Passalora fulva, Cladosporium oxysporum, Dothistroma septosporum, Isariopsis clavispora, Mycosphaerella fijiensis, Mycosphaerella graminicola, Mycovellosiella koepkeii, Phaeoisariopsis bataticola, Pseudocercospora vitis, Pseudocercosporella herpotrichoides, Ramularia beticola, Ramularia collo-cygni, Magnaporthales such as Gaeumannomyces graminis, Magnaporthe grisea, Pyricularia oryzae, Diaporthales such as Anisogramma anomala, Apiognomonia errabunda, Cytospora platani, Diaporthe phaseolorum, Discula destructiva, Gnomonia fructicola, Greeneria uvicola, Melanconium juglandinum, Phomopsis viticola, Sirococcus clavigignenti-juglandacearum, Tubakia dryina, Dicarpella spp., Valsa ceratosperma, and others such as Actinothyrium graminis, Ascochyta pisi, Aspergillus flavus, Aspergillus fumigatus, Aspergillus nidulans, Asperisporium caricae, Blumeriella jaapii, Candida spp., Capnodium ramosum, Cephaloascus spp., Cephalosporium gramineum, Ceratocystis paradoxa, Chaetomium spp., Hymenoscyphus pseudoalbidus, Coccidioides spp., Cylindrosporium padi, Diplocarpon malae, Drepanopeziza campestris, Elsinoe ampelina, Epicoccum nigrum, Epidermophyton spp., Eutypa lata, Geotrichum candidum, Gibellina cerealis, Gloeocercospora sorghi, Gloeodes pomigena, Gloeosporium perennans; Gloeotinia temulenta, Griphospaeria corticola, Kabatiella lini, Leptographium microsporum, Leptosphaerulinia crassiasca, Lophodermium seditiosum, Marssonina graminicola, Microdochium nivale, Monilinia fructicola, Monographella albescens, Monosporascus cannonballus, Naemacyclus spp., Ophiostoma novo-ulmi, Paracoccidioides brasiliensis, Penicillium expansum, Pestalotia rhododendri, Petriellidium spp., Pezicula spp., Phialophora gregata, Phyllachora pomigena, Phymatotrichum omnivora, Physalospora abdita, Plectosporium tabacinum, Polyscytalum pustulans, Pseudopeziza medicaginis, Pyrenopeziza brassicae, Ramulispora sorghi, Rhabdocline pseudotsugae, Rhynchosporium secalis, Sacrocladium oryzae, Scedosporium spp., Schizothyrium pomi, Sclerotinia sclerotiorum, Sclerotinia minor; Sclerotium spp., Typhula ishikariensis, Seimatosporium mariae, Lepteutypa cupressi, Septocyta ruborum, Sphaceloma perseae, Sporonema phacidioides, Stigmina palmivora, Tapesia yallundae, Taphrina bullata, Thielviopsis basicola, Trichoseptoria fructigena, Zygophiala jamaicensis; powdery mildew diseases for example those caused by Erysiphales such as Blumeria graminis, Erysiphe polygoni, Uncinula necator, Sphaerotheca fuligena, Podosphaera leucotricha, Podospaera macularis Golovinomyces cichoracearum, Leveillula taurica, Microsphaera diffusa, Oidiopsis gossypii, Phyllactinia guttata and Oidium arachidis; molds for example those caused by Botryosphaeriales such as Dothiorella aromatica, Diplodia seriata, Guignardia bidwellii, Botrytis cinerea, Botryotinia allii, Botryotinia fabae, Fusicoccum amygdali, Lasiodiplodia theobromae, Macrophoma theicola, Macrophomina phaseolina, Phyllosticta cucurbitacearum; anthracnoses for example those caused by Glommerelales such as Colletotrichum gloeosporioides, Colletotrichum lagenarium, Colletotrichum gossypii, Glomerella cingulata, and Colletotrichum graminicola; and wilts or blights for example those caused by Hypocreales such as Acremonium strictum, Claviceps purpurea, Fusarium culmorum, Fusarium graminearum, Fusarium virguliforme, Fusarium oxysporum, Fusarium subglutinans, Fusarium oxysporum f.sp. cubense, Gerlachia nivale, Gibberella fujikuroi, Gibberella zeae, Gliocladium spp., Myrothecium verrucaria, Nectria ramulariae, Trichoderma viride, Trichothecium roseum, and Verticillium theobromae;

Basidiomycetes, including smuts for example those caused by Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries;

Blastocladiomycetes, such as Physoderma maydis;

Mucoromycetes, such as Choanephora cucurbitarum.; Mucor spp.; Rhizopus arrhizus;

as well as diseases caused by other species and genera closely related to those listed above.

In addition to their fungicidal activity, the compositions may also have activity against bacteria such as Erwinia amylovora, Erwinia caratovora, Xanthomonas campestris, Pseudomonas syringae, Strptomyces scabies and other related species as well as certain protozoa.

The composition according to the invention is particularly effective against phytopathogenic fungi belonging to the following classes: Ascomycetes (e.g. Venturia, Podosphaera, Erysiphe, Monilinia, Mycosphaerella, Uncinula); Basidiomycetes (e.g. the genus Hemileia, Rhizoctonia, Phakopsora, Puccinia, Ustilago, Tilletia); Fungi imperfecti (also known as Deuteromycetes; e.g. Botrytis, Helminthosporium, Rhynchosporium, Fusarium, Septoria, Cercospora, Alternaria, Pyricularia and Pseudocercosporella); Oomycetes (e.g. Phytophthora, Peronospora, Pseudoperonospora, Albugo, Bremia, Pythium, Pseudosclerospora, Plasmopara).

In preferred embodiments, the following inventive polymorphs of compounds of formula (I-A) and (I-B) and mixtures thereof, as listed above, can be used on the following crops and pests:

Preferably, the inventive polymorphs of compounds of formula (I-A) and (I-B) and mixtures thereof, as listed above, are suitable for controlling the following fungal diseases on soybeans and genetically modified soybeans, for example Bt soybeans: Alternaria spp. (Alternaria leaf spot); Cercospora spp. (Cercospora leaf spots), e.g., C. sojina or C. kikuchii Colletotrichum (teleomorph: Glomerella) spp. (anthracnose), e.g., C. truncatum or C. gloeosporioides)′, Corynespora cassiicola (leaf spots); Dematophora (teleomorph: Rosellinia) necatrix (root and stem rot); Diaporthe spp., e.g., D. phaseolorum (damping off); Fusarium (teleomorph: Gibberella) spp. (wilt, root or stem rot), e.g. F. tucumaniae and F. brasiliense each causing sudden death syndrome on soybeans; Macrophomina phaseolina (syn. phaseoli) (root and stem rot); Microsphaera diffusa (powdery mildew); Peronospora spp. (downy mildew), e.g., P. manshurica; Phakopsora pachyrhizi and P. meibomiae (soybean rust); Phialophora spp., e.g., P. g reg ata′. stem rot; Phomopsis spp., e.g., stem rot: P. phaseoli (teleomorph: Diaporthe phaseolorum)′, Pythium spp. (damping-off); Phytophthora spp. (wilt, root, leaf, fruit and stem root), e.g., P. megasperma, syn. P. sojae)′, Rhizoctonia spp., e.g., R. solani (root and stem rot); Sclerotinia spp. (stem rot or white mold); Septoria spp., e.g., S. glycines (brown spot); S. rolfsii (syn. Athelia rolfsii); Thielaviopsis spp. (black root rot).

More preferably, the inventive polymorphs of compounds of formula (I-A) and (I-B) and mixtures thereof, as listed above, are suitable for controlling the following fungal diseases on soybeans and genetically modified soybeans, for example Bt soybeans: Alternaria spp. (Alternaria leaf spot); Cercospora spp. (Cercospora leaf spots), e.g., C. sojina or C. kikuchii); Colletotrichum (teleomorph: Glomerella) spp. (anthracnose), e.g., C. truncatum or C. gloeosporioides); Corynespora cassiicola (leaf spots); Diaporthe spp., e.g., D. phaseolorum (damping off); Fusarium (teleomorph: Gibberella) spp. (wilt, root or stem rot), e.g. F. tucumaniae and F. brasiliense each causing sudden death syndrome on soybeans; Macrophomina phaseolina (syn. phaseoli) (root and stem rot); Peronospora spp. (downy mildew), e.g., P. manshurica; Phakopsora pachyrhizi and P. meibomiae (soybean rust); Phomopsis spp., e.g., stem rot: P. phaseoli (teleomorph: Diaporthe phaseolorum); Phytophthora spp. (wilt, root, leaf, fruit and stem root), e.g., P. megasperma, syn. P. sojae); Rhizoctonia spp., e.g., R. solani (root and stem rot); Septoria spp., e.g., S. glycines (brown spot).

The polymorphs of compounds of formula (I-A) and (I-B) and mixtures thereof, as listed above, according to the present invention are particularly important for controlling phyto-pathogenic harmful fungi on soybeans and genetically modified soybeans, for example Bt soybeans.

The polymorphs of compounds of formula (I-A) and (I-B) and mixtures thereof, as listed above, according to the present invention are also particularly important for controlling Phakopsora pachyrhizi, P. meibomiae (soybean rust), Cercospora sojina, Cercospora kikuchii, Corynospera cassiicula, Colletotrichum truncatum, Sclerotinia sclerotiorum, Microsphaera diffusa, Septoria glycine, Peronospora manshurica or Diaporthe caulivora (D. phaseolorum var. Caulivora), in each case on soybeans and genetically modified soybeans, for example Bt soybeans.

In preferred embodiments, the following of compounds of formula (I-A) and (I-B) and mixtures thereof, as listed above, can be used on the following crops and pests:

Preferably, the of formula (I-A) and (I-B) and mixtures thereof, as listed above, are suitable for controlling the following fungal diseases on soybeans and genetically modified soybeans, for example Bt soybeans: Alternaria spp. (Alternaria leaf spot); Cercospora spp. (Cercospora leaf spots), e.g., C. sojina or C. kikuchii Colletotrichum (teleomorph: Glomerella) spp. (anthracnose), e.g., C. truncatum or C. gloeosporioides)′, Corynespora cassiicola (leaf spots); Dematophora (teleomorph: Rosellinia) necatrix (root and stem rot); Diaporthe spp., e.g., D. phaseolorum (damping off); Fusarium (teleomorph: Gibberella) spp. (wilt, root or stem rot), e.g., F. tucumaniae and F. brasiliense each causing sudden death syndrome on soybeans; Macrophomina phaseolina (syn. phaseoli) (root and stem rot); Microsphaera diffusa (powdery mildew); Peronospora spp. (downy mildew), e.g., P. manshurica; Phakopsora pachyrhizi and P. meibomiae (soybean rust); Phialophora spp., e.g., P. g reg ata′. stem rot; Phomopsis spp., e.g., stem rot: P. phaseoli (teleomorph: Diaporthe phaseolorum)′, Pythium spp. (damping-off); Phytophthora spp. (wilt, root, leaf, fruit and stem root), e.g., P. megasperma, syn. P. sojae)′, Rhizoctonia spp., e.g., R. solani (root and stem rot); Sclerotinia spp. (stem rot or white mold); Septoria spp., e.g., S. glycines (brown spot); S. rolfsii (syn. Athelia rolfsii); Thielaviopsis spp. (black root rot).

More preferably, the compounds of formula (I-A) and (I-B) and mixtures thereof, as listed above, are suitable for controlling the following fungal diseases on soybeans and genetically modified soybeans, for example Bt soybeans: Alternaria spp. (Alternaria leaf spot); Cercospora spp. (Cercospora leaf spots), e.g., C. sojina or C. kikuchii); Colletotrichum (teleomorph: Glomerella) spp. (anthracnose), e.g., C. truncatum or C. gloeosporioides); Corynespora cassiicola (leaf spots); Diaporthe spp., e.g., D. phaseolorum (damping off); Fusarium (teleomorph: Gibberella) spp. (wilt, root or stem rot), e.g. F. tucumaniae and F. brasiliense each causing sudden death syndrome on soybeans; Macrophomina phaseolina (syn. phaseoli) (root and stem rot); Peronospora spp. (downy mildew), e.g., P. manshurica; Phakopsora pachyrhizi and P. meibomiae (soybean rust); Phomopsis spp., e.g., stem rot: P. phaseoli (teleomorph: Diaporthe phaseolorum); Phytophthora spp. (wilt, root, leaf, fruit and stem root), e.g., P. megasperma, syn. P. sojae); Rhizoctonia spp., e.g., R. solani (root and stem rot); Septoria spp., e.g., S. glycines (brown spot).

The compounds of formula (I-A) and (I-B) and mixtures thereof, as listed above, according to the present invention are particularly important for controlling phyto-pathogenic harmful fungi on soybeans and genetically modified soybeans, for example Bt soybeans.

The compounds of formula (I-A) and (I-B) and mixtures thereof, as listed above, according to the present invention are also particularly important for controlling Phakopsora pachyrhizi, P. meibomiae (soybean rust), Cercospora sojina, Cercospora kikuchii, Corynospera cassiicula, Colletotrichum truncatum, Sclerotinia sclerotiorum, Microsphaera diffusa, Septoria glycine, Peronospora manshurica or Diaporthe caulivora (D. phaseolorum var. Caulivora), in each case on soybeans and genetically modified soybeans, for example Bt soybeans.

The polymorph of compound of formula (I-A) or (I-B) according to the invention may be used for example on turf, ornamentals, such as flowers, shrubs, broad-leaved trees, or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g., imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names Roundup Ready®, Herculex I* and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink* (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT* (cotton variety that expresses a VIP toxin); NewLeaf® (potato variety that expresses a CryIIIA toxin); Nature-Gard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed using recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as delta-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by delta-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 2002/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 2003/018810).

Examples of such toxins or transgenic plants capable of synthesising such toxins are disclosed, for example, in EP-A-0 374 753, WO1993/07278, WO1995/34656, EP-A-0 427 529, EP-A-451 878 and WO2003/052073.

The processes for the preparation of such transgenic plants are generally known to a person skilled in the art and are described, for example, in the publications mentioned above. CryI-type deoxyribonucleic acids and their preparation are known, for example, from WO1995/34656, EP-A-0 367 474, EP-A-0 401 979 and WO1990/13651.

The toxin contained in the transgenic plants imparts to the plants tolerance to harmful insects. Such insects can occur in any taxonomic group of insects but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1 Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard 1® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 2003/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B 1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Preferably, the polymorphs of the invention according to the first or second aspect or fungicidal compositions according to the present invention comprising said polymorphs may be used in controlling or preventing phytopathogenic diseases, especially phytopathogenic fungi (such as Phakopsora pachyrhizi) on soybean plants.

In particular, transgenic soybean plants expressing toxins, for example insecticidal proteins such as delta-endotoxins, e.g., Cry1Ac (Cry1Ac Bt protein). Accordingly, this may include transgenic soybean plants comprising event MON87701 (disclosed in WO2009/064652), event MON87701×MON89788 (disclosed in WO2014/170327, e.g. commercially available as Intacta RR2 PRO® soybean), event MON87751 (disclosed in WO2014/201235), event DAS-44406-6 (e.g., commercially available as Enlist E3™, DAS-44406-6, disclosed in WO2012/075426), or event DAS-81419-2 (described in WO2013/016527, e.g., commercially available as Conkesta™ soybean); event DAS-81419-2×DAS-44406-6 (e.g., commercially available as Conkesta™ Enlist E3™ Soybean).

Useful transgenic events in transgenic soybean plants, which can be treated according to the invention, include event DAS-44406-6/pDAB8264.44.06.1 (soybean, herbicide-tolerance, disclosed in WO2012/075426); event DAS-81419-2 (described in WO2013/016527 (e.g., commercially available as aka Conkesta™ soybean, Conkesta™ Enlist E3™ soybean, DAS-81419-2×DAS-44406-6); event DAS-14536-7/pDAB8291.45.36.2 (soybean, herbicide-tolerance, disclosed in WO2012/075429); DAS-68416-4 (soybean, herbicide-tolerance, ATCC Accession No. PTA-10442, disclosed in WO2011/066384, WO2011/066360); event DP-305423-1 (soybean, quality mark, disclosed in WO2008/054747, e.g. commercially available as Treus™, Plenish™, Plenish® High Oleic Soybeans); event DP-356043-5 (soybean, herbicide-tolerance, deposited as ATCC PTA-8287, disclosed in WO2008/002872, e.g. commercially available as Optimum GAT™); event FG72 (soybean, herbicide-tolerance, disclosed in WO2011/063413); event LL27 (soybean, herbicide-tolerance, disclosed in WO2006/108674); event LL55 (soybean, herbicide-tolerance, disclosed in WO 2006/108675); event EE-GM3/FG72 (soybean, herbicide-tolerance) optionally stacked with event EE-GM1/LL27 or event EE-GM2/LL55 (disclosed in WO2011/063413); event MON87701 (soybean, insect control, disclosed in WO2009/064652, WO2014/170327); event MON87701×MON89788 (disclosed in WO2014/170327, e.g. commercially available as Intacta RR2 PRO® soybean); event MON87705 (soybean, improved fatty acid profile, herbicide-tolerance, disclosed in WO2010/037016 or US20100080887A, e.g. commercially available as Vistive Gold™); event MON87751 (lepidopteran-resistant, ATCC accession no. PTA-120166. disclosed in WO2014/201235); event MON87751×MON87701×MON89788×MON87708 (commercially available as Intacta2 Xtend®); event MON87708 (soybean, herbicide-tolerance, disclosed in WO2011/034704, e.g. commercially available as Genuity® Roundup Ready™ 2 Xtend™); event MON87708×MON89788 (soybean, e.g. commercially available as Roundup Ready™ 2 Xtend™); event MON87712 (soybean, yield, disclosed in WO2012/051199); event MON87754 (soybean, quality feature, disclosed in WO2010/024976); event MON87769 (soybean, quality attribute, disclosed in WO2009/102873); event MON89788 (soybean, herbicide-tolerance, disclosed in WO2006/130436, e.g. commercially available as Genuity® Roundup Ready 2 Yield™); event SYHT0H2/SYN-000H2-5 (soybean, herbicide-tolerance, disclosed in WO2012/082548); event DAS-21606-3 (soybean, herbicide-tolerance, disclosed in WO2012/033794); event 8264.44.06.1 (soybean, stacked herbicide-tolerance, disclosed in W02022/012075426); event pDAB8291.45.36.2 (soybean, stacked herbicide-tolerance, disclosed in WO2012/075429); event pDAB8264.42.32.1 (soybean, stacked herbicide-tolerance, disclosed in WO2013/010094); event A2704-12 (glufosinate tolerance, disclosed in WO2006/108647); event A5547-127 (phosphinothricin tolerant, disclosed in WO2006/108675); event BPS-CV127-91 (herbicide tolerance, disclosed in WO 2010/080829); event GU262 (phosphinothricin tolerant, described in APHIS regulatory reference US 98-238-01p); event MON 87708×MON 89788×A5547-127; G72×A5547-127 (event code: MST-FGØ72-3×ACS-GMØØ6-4, e.g. commercially available as Liberty Link™ soybean), event MON-04032-6 (event code: GTS 40-3-2, http://www.agbios.com/static/cropdb/LONG-GTS-40-3-2-printer.html. e.g. commercially available as Roundup Ready® soybean), event HB4 (event code IND-00410-5, US2022/009011, e.g., commercially available as Verdeca HB4 Soybean).

Particularly useful transgenic events in transgenic soybean plants, which can preferably be treated according to the invention, include event A2704-12 (glufosinate tolerance, disclosed in WO2006/108647, e.g., commercially available as Liberty Link™ soybean), event A5547-127 (phosphinothricin tolerant, disclosed in WO2006/108675, U.S. Pat. No. 8,952,142B2, e.g., commercially available as Liberty Link™ soybean); A5547-35 (event code: ACS-GMØØ8-6, gene: pat, e.g. commercially available as Liberty Link™ soybean), event MON89788 (soybean, herbicide-tolerance, disclosed in WO2006/130436, e.g. commercially available as Genuity® Roundup Ready 2 Yield™); DP-305423-1 (soybean, quality mark, disclosed in WO2008/054747, e.g., commercially available as Treus™, Plenish™, Plenish® High Oleic Soybeans); event MON87701 (soybean, insect resistant, disclosed in WO2009/064652); event MON87701×MON89788 (disclosed in WO2014/170327, e.g. commercially available as Intacta RR2 PRO® soybean); event MON87705 (soybean, improved fatty acid profile, herbicide-tolerance, disclosed in WO2010/037016 or US20100080887A, e.g. commercially available as Vistive Gold™); event FG72 (soybean, herbicide-tolerance, disclosed in WO2011/063413); evet FG72×A5547-127 (e.g. commercially available as LibertyLink® GT27™); event SYHT0H2/SYN-000H2-5 (soybean, herbicide-tolerance, disclosed in WO2012/082548); event DAS-81419-2 (described in WO2013/016527, e.g., commercially available as Conkesta™ soybean); event DAS-44406-6 (disclosed in WO2012/075426, e.g., commercially available as Enlist E3™), or event DAS-81419-2 (described in WO2013/016527, e.g., commercially available as Conkesta™ soybean); DAS81419-2×DAS4406 (e.g., commercially available as Conkesta™ Enlist E3™ soybean); event DP305423×GTS 40-3-2 (soybean, quality mark, disclosed in WO2008/054747, e.g. commercially available as Plenish® High Oleic Soybeans); event DP-356043-5 (soybean, herbicide-tolerance, deposited as ATCC PTA-8287, disclosed in WO2008/002872, e.g. commercially available as Optimum GAT™), event MON-04032-6 (event code: GTS 40-3-2, http://www.agbios.com/static/cropdb/LONG-GTS-40-3-2-printer.html, e.g. commercially available as Roundup Ready® soybean).

Furthermore, such a list of transgenic events is provided by the United States Department of Agriculture's (USDA) Animal and Plant Health Inspection Service (APHIS) and can be found on their website on the World Wide Web at aphis.usda.gov.

Commercially available examples of genetically modified soybean plants, which can preferably be treated according to the invention, include commercially available products such as plant seeds, which are under the Roundup Ready® (RR1), Roundup Ready 2 Xtend®, Roundup Ready 2 Yield®, XtendFlex®, Intacta® Roundup Ready™ 2 Pro (Intacta® RR2 PRO), Intacta 2 Xtend®, Vistive® Gold™, Conkesta Enlist E3® Conkesta E3®, Enlist E3®, Genuity® Roundup Ready 2 Yield™, Genuity® Roundup Ready™ 2 Xtend™ Herbicide-tolerant Soybean line, Optimum GAT™, Liberty Link™ Soybean, Verdeca HB4 Soybean, Treus™, Plenish™ trade names sold or distributed.

Transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS81419-2, GU262, SYHTØH2, W62, W98, FG72 and CV127.

According to one embodiment of the invention, there is provided the use of a compound of formula (I-A) or the polymorph thereof (I-A) for controlling phytopathogenic fungi in genetically modified soybean plants, wherein said Transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS81419-2, GU262, SYHTØH2, W62, W98, FG72 and CV127.

According to one embodiment of the invention, there is provided the use of a compound of formula (I-B) or the polymorph thereof (I-B) for controlling phytopathogenic fungi in genetically modified soybean plants, wherein said Transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS81419-2, GU262, SYHTØH2, W62, W98, FG72 and CV127.

The polymorphs of the invention according to the first or second aspect or fungicidal compositions according to the present invention comprising said polymorphs may be used in controlling or preventing phytopathogenic diseases, especially phytopathogenic fungi (such as Phakopsora pachyrhizi) on soybean plants. In particular, there are known in the scientific literature certain Elite soybean plant varieties where R-gene stacks, conferring a degree of immunity or resistance to specific Phakopsora pachyrhizi, have been been introgressed in the plant genome, see for example: “Fighting Asian Soybean Rust”, Langenbach C, et al, Front Plant Science 7(797) 2016).

An elite plant is any plant from an elite line, such that an elite plant is a representative plant from an elite variety. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9; S52-Y2; S58-Z4; S67-R6; S73-S8; and S78-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

Thus, in a further preferred embodiment, the polymorphs of the invention (I-A) or (I-B), according to the first or second aspect or fungicidal compositions according to the present invention comprising said polymorphs (I-A) or (I-B), are used to control Phakopsora pachyrhizi, (including fungicidally-resistant strains thereof, as outlined below) on Elite soybean plant varieties where R-gene stacks, conferring a degree of immunity or resistance to specific Phakopsora pachyrhizi, have been been introgressed in the plant genome. Numerous benefits may be expected to ensue from said use, e.g. improved biological activity, an advantageous or broader spectrum of activity (inc. sensitive and resistant strains of Phakopsora pachyrhizi), an increased safety profile, improved crop tolerance, synergistic interactions or potentiating properties, improved onset of action or a longer lasting residual activity, a reduction in the number of applications and/or a reduction in the application rate of the compounds and compositions required for effective control of the phytopathogen (Phakopsora pachyrhizi), thereby enabling beneficial resistance-management practices, reduced environmental impact and reduced operator exposure.

Under certain circumstances, fungicidal compositions according to the present invention comprising a polymorph of formula (I-A) or (I-B) when used in controlling or preventing phytopathogenic diseases, especially phytopathogenic fungi (such as Phakopsora pachyrhizi) on soybean plants (in particular any of the transgenic soybean plants as described above), may display a synergistic interaction between the active ingredients.

Exemplary GM traits that confer enhanced ASR resistance comprise resistance genes encoding resistance proteins as set forth in: WO2019103918 (for example, but not limited to, RG-1 (SEQ ID NO: 47) and active variants or fragments thereof; or R-genes as set forth at SEQ ID NO: 28, 42, 43, 44, 45 or 46 of WO2019103918); WO202100878 (for example Rpp6907 (SEQ ID NO: 1 of WO202100878) and active variants or fragments thereof); WO2021022022 (for example, TirA or Tir B (SEQ ID NOS: 11 or 16 of WO2021022022, respectively) or active variants or fragments thereof); WO2021260673 (for example, but not limited to, RG21 and/or RG22 (SEQ ID NOS: 1 or 12 of WO2021260673) or active variants or fragments thereof); WO2022173659 (for example, but not limited to, RG30 (SEQ ID NO: 5 of WO2022173659) or active variants or fragments thereof); WO2022159341 (for example but not limited to SEQ ID NOS: 1 and 148 of WO2022159341 or active variants or fragments thereof); WO2021154632A1, WO2021022026, WO2021022101, US20220135997 (for example, but not limited to, FIT1 (SEQ ID NO: 2 of US20220135997), an active variant or fragment thereof or any of the FIT1 paralogs or orthologs disclosed therein (such as SEQ ID NOS: 4, 6, 8, 10, 12, 14, 16, 18 or 20 of US20220135997); U.S. Ser. No. 10/842,097 (for example, but not limited to, CcRpp1 or active variants or fragments thereof or any other resistance genes disclosed therein); WO2022140257 (for example, CcRpp2-R1 and/or CcRpp2-R3 (SEQ ID NOS: 2 or 4 of WO2022140257) or an active variant or fragment thereof); the resistance genes disclosed in U.S. Provisional Application No. 63/481,627 as RG31 (SEQ ID NOS: 1, 3, or 4) or RG35 (SEQ ID NOS: 2 or 5); and/or the resistance genes disclosed in U.S. Provisional Application Nos. 63/426,524 and 63/509,586 as RG32 (SEQ ID NOS: 1, 3, or 4) or RG34 (SEQ ID NOS: 2, 5, 6, or 17); each of which is incorporated by reference in their entirety.

Exemplary native traits that confer increased resistance to ASR or to pathogens from the genus Phakopsora, including the species Phakopsora pachyrhizi and Phakopsora meibomiae include various intervals and locus (loci) associated with Rpp1, Rpp1b, Rpp2, Rpp4, Rpp5, Rpp6 and ASR resistance locus 1-16. Such native traits can be found, for example, in WO2009079729, U.S. Pat. Nos. 8,759,607, 8,962,914, WO2008054546, U.S. Pat. Nos. 8,692,054, 9,091,681, WO2009132089, U.S. Pat. Nos. 8,669,414, 8,796,503, 8,921,645, WO2010096227, WO2010009404, WO2021154632, US20230067451, WO2021022026, US20220256795, WO2021022101, US20220338433A1, WO2022173659, WO2010009404, WO2017222827, US20210024950, WO2021000878, US20220380796, US20230147114, and PCT App. No. PCT/US23/60373, each of which is incorporated by reference in their entirety.

Exemplary Soybean varieties that confer increased resistance to ASR include soybean cultivars TMG 7062, TMG 7161 and TMG 7261.

Further Soybean varieties that confer increased resistance against ASR (caused by Phakopsora pachyrhizi) inlcude for example, but not limited to TMG7368 IPRO (Disclosed in WO2009079729), TMG7062 IPRO, TMG 7063 IPRO, and TMG 7061 IPRO.

Further Soybean varieties that confer increased resistance against ASR (caused by Phakopsora pachyrhizi) inlcude for example, but not limited to soybeans with Shield Technology, like for example BRS511 soybean, BRS 531 soybean, or Soy-BRS 539 (conventional soybean with Shield® and Block® Technologies).

The polymorphs of the invention according to the first or second aspect or fungicidal compositions according to the present invention comprising said polymorphs may be used in controlling or preventing phytopathogenic diseases, especially phytopathogenic fungi (in particular, Phakopsora pachyrhizi) on soybean plants.

Additionally, to date, no cross-resistance has been observed between the said polymorphs and the current fungicidal solutions used to control Phakopsora pachyrhizi.

Indeed, fungicidal-resistant strains of Phakopsora pachyrhizi have been reported in the scientific literature, with strains resistant to one or more fungicides from at least each of the following fungicidal mode of action classes being observed: sterol demethylation-inhibitors (DMI), quinone-outside-inhibitors (QoI) and succinate dehydrogenase inhibitors (SDHI). See for example: “Sensitivity of Phakopsora pachyrhizi towards quinone-outside-inhibitors and demethylation-inhibitors, and corresponding resistance mechanisms.” Schmitz H K et al, Pest Manag Sci (2014) 70: 378-388; “First detection of a SDH variant with reduced SDHI sensitivity in Phakopsora pachyrhizi” Simões K et al, J Plant Dis Prot (2018) 125: 21-2; “Competitive fitness of Phakopsora pachyrhizi isolates with mutations in the CYP51 and CYTB genes.” Klosowski A C et al, Phytopathology (2016) 106: 1278-1284; “Detection of the F129L mutation in the cytochrome b gene in Phakopsora pachyrhizi.” Klosowski A C et al, Pest Manag Sci (2016) 72:1211-1215.

Thus, in a preferred embodiment, the polymorphs of the invention according to the first or second aspect or fungicidal compositions according to the present invention comprising said polymorphs, are used to control Phakopsora pachyrhizi which are resistant to one or more fungicides from any of the following fungicidal MoA classes: sterol demethylation-inhibitors (DMI), quinone-outside-inhibitors (QoI) and succinate dehydrogenase inhibitors (SDHI).

The polymorph of compound of formula (I-A) or (I-B) according to the invention may be used in controlling or preventing phytopathogenic diseases, especially phytopathogenic fungi such as Alternaria species in fruits, vegetables and potatoes; Botrytis cinerea in strawberries, tomatoes, sunflower, pulse crops, vegetables and grapes; Rhizoctonia solani in potatoes and vegetables; Uncinula necator in grapes; Cladosporium cucumerinum, Didymella bryoniae, Sphaerotheca fuliginea and Glomerella lagenarium in cucurbits; Leveillula taurica in cucurbits and solanacious crops; Fusarium spp. in cereals; Leptosphaeria spp. in cereals; and Zymospetoria spp. in cereals.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The polymorph of compound of formula (I-A) or (I-B) according to the invention may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g., in polymeric substances. As with the type of the compositions, the methods of application, such as spraying, atomising, dusting, scattering, coating, or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g., for agricultural use, can be solid or liquid and are substances useful in formulation technology, e.g., natural or regenerated mineral substances, solvents, dispersants, wetting agents, tackifiers, thickeners, binders or fertilizers. Such carriers are for example described in WO 1997/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours, and other organic and inorganic solids which act as dispersants and carriers.

The active ingredients (compounds of formula (I-A) or (I-B) and mixtures thereof with component (B) can also be contained in microcapsules. Microcapsules contain the active ingredients in a porous carrier. This enables the active ingredients to be released into the environment in controlled amounts (e.g., slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane, or chemically modified polymers, and starch xanthates, or other polymers that are known to the person skilled in the art. Alternatively, very fine microcapsules can be formed in which the active ingredient is contained in the form of finely divided particles in a solid matrix of base substance, but the microcapsules are not themselves encapsulated.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust, and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely divided form because of vaporisation of a low boiling dispersant solvent carrier, may also be used.

Suitable agricultural adjuvants and carriers that are useful in formulating the compositions of the invention in the formulation types described above are well known to a person skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface-active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidal active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidal active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The polymorph of compound of formula (I-A) or (I-B) according to the invention are normally used in the form of agrochemical compositions and can be applied to the crop area or plant to be treated, simultaneously or in succession with further compounds. These further compounds can be e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The polymorph of compound of formula (I-A) or (I-B) according to the invention may be used in the form of (fungicidal) compositions for controlling or protecting against phytopathogenic microorganisms, comprising as active ingredient a polymorph as defined herein, in free form or in agrochemical usable salt form, and at least one of the above-mentioned adjuvants.

The invention therefore provides a composition, preferably a fungicidal composition, comprising a polymorph of compound of formula (I-A) or (I-B) according to the invention, an agriculturally acceptable carrier and optionally an adjuvant. An agricultural acceptable carrier is for example a carrier that is suitable for agricultural use. Agricultural carriers are well known in the art. Preferably, said composition may comprise at least one or more pesticidal-active compounds, for example an additional fungicidal active ingredient in addition to the polymorph of compound of formula (I-A) or (I-B).

The polymorph of compound of formula (I-A) or (I-B) according to the invention may be the sole active ingredient of a composition or it may be admixed with one or more additional active ingredients such as a pesticide, fungicide, synergist, herbicide, or plant growth regulator where appropriate. An additional active ingredient may, in some cases, result in unexpected synergistic activities.

Examples of suitable additional active ingredients include the following: acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients include the following: petroleum oils, 1,1-bis(4-chlorophenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxa-fos, benzyl benzoate, bixafen, brofenvalerate, bromocyclen, bromophos, bromopropylate, buprofezin, butocarboxim, butoxycarboxim, butylpyridaben, calcium polysulfide, camphechlor, carbanolate, carbophenothion, cymiazole, chinomethionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dinopenton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fenpyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8, 10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B1, trimedlure B2, trimedlure C, trunc-call, 2-(octylthio)ethanol, butopyronoxyl, butoxy(polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)ethane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethylcarbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, 0-5-dichloro-4-iodophenyl O-ethyl ethylphosphonothioate, O,O-diethyl O-4-methyl-2-oxo-2H-chromen-7-yl phosphorothioate, O,O-diethyl 0-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla, schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothiophene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin, 2-(2-butoxyethoxy)ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, sulphur, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil, imibenconazole, ipconazole, metconazole, myclobutanil, paclobutrazole, pefurazoate, penconazole, prothioconazole, pyrifenox, prochloraz, propiconazole, pyrisoxazole, simeconazole, tebuconazole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline, procymidone, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chlorothalonil, cyflufenamid, cymoxanil, cyclobutrifluram, diclocymet, diclomezine, dicloran, diethofencarb, dimethomorph, flumorph, dithianon, ethaboxam, etridiazole, famoxadone, fenamidone, fenoxanil, ferimzone, fluazinam, flumetylsulforim, fluopicolide, fluoxytioconazole, flusulfamide, fluxapyroxad, fenhexamid, fosetylaluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 2,4-D (2,4-dichlorophenoxyacetic acid), 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1, 3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl)quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, metarylpicoxamid, tebufloquin, ipflufenoquin, quinofumelin, isofetamid, ethyl 1-[[4-[[2-(trifluoromethyl)-1,3-dioxolan-2-yl]methoxy]phenyl]methyl]pyrazole-3-carboxylate (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate (may be prepared from the methods described in WO 2020/056090), methyl N-[[4-[1-(4-cyclopropyl-2,6-difluoro-phenyl)pyrazol-4-yl]-2-methyl-phenyl]methyl]carbamate (may be prepared from the methods described in WO 2020/097012), methyl N-[[4-[1-(2,6-difluoro-4-isopropyl-phenyl)pyrazol-4-yl]-2-methyl-phenyl]methyl]carbamate (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391), N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flufenoxadiazam, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1,1,3-trimethylindan-4-yl) pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, α-(1,1-dimethylethyl)-α-[4′-(trifluoromethoxy) [1,1′-biphenyl]-4-yl]-5-pyrimidinemethanol, fluoxapiprolin, enoxastrobin, 4-[[6-[2-(2,4-difluorophenyl)-1,1-difluoro-2-hydroxy-3-(1,2,4-triazol-1-yl)propyl]-3-pyridyl]oxy]benzonitrile, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentanecarboxylate, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac-ethyl, coumoxystrobin, zhongshengmycin, thiodiazole copper, zinc thiazole, amectotractin, iprodione, seboctylamine, thiamethoxam, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO 2018/228896); N-ethyl-N′-[5-methoxy-2-methyl-4-[(2-trifluoromethyl)oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl)isoquinoline, 4,4-difluoro-3,3-dimethyl-1-(7-methylpyrazolo[1,5-a]pyridin-3-yl)isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085), N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxy)-2-(trifluoromethyl)-3-pyridyl]-1-(1,2,4-triazol-1-yl)propan-2-ol (this compound may be prepared from the methods described in WO2017/029179); 2-[6-(4-bromophenoxy)-2-(trifluoromethyl)-3-pyridyl]-1-(1,2,4-triazol-1-yl)propan-2-ol (this compound may be prepared from the methods described in WO2017/029179); 3-[2-(1-chlorocyclopropyl)-3-(2-fluorophenyl)-2-hydroxy-propyl]imidazole-4-carbonitrile (this compound may be prepared from the methods described in WO2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO2014/006945); 2,6-Dimethyl-1H,5H-[1,4]dithiino[2,3-c:5,6-c′]dipyrrole-1,3,5,7(2H,6H)-tetrone (this compound may be prepared from the methods described in WO2011/138281)N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO2017/220485); 2-oxo-N-propyl-2-[4-[5-(trifluoromethyl)-1,2,4-oxadiazol-3-yl]phenyl]acetamide (this compound may be prepared from the methods described in WO2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N—[N-methoxy-methyl-carbonimidoyl]-4-[5-(trifluoromethyl)-1,2,4-oxadiazol-3-yl]benzamide (these compounds may be prepared from the methods described in WO 2018/202428); 1-[1-(4-chloro phenyl)cyclobutyl]ethyl (2S)-2-[(3-hydroxy-4-methoxy-pyridine-2-carbonyl)-amino]propanoate; [2-(4-bromo-7-fluoro-indol-1-yl)-1-methyl-propyl](2S)-2-[(3-hydroxy-4-methoxy-pyridine-2-carbonyl]amino]propanoate; [2-(3,5-dichloro-2-pyridyl)-1-methyl-propyl](2S)-2-[(3-hydroxy-4-methoxy-pyridine-2-carbonyl)amino]propanoate; [(1 S)-1-[1-(1-naphthyl)cyclopropyl]ethyl](2S)-2-[[3-(acetoxymethoxy)-4-methoxy-pyridine-2-carbonyl]amino]propanoate, [(1S)-1-[1-(1-naphthyl)cyclopropyl]ethyl](2S)-2-[(3-hydroxy-4-methoxy-pyridine-2-carbonyl)amino]propanoate, [2-[[(1S)-2-[2-(3,5-dichloro-2-pyridyl)-1-methyl-propoxy]-1-methyl-2-oxo-ethyl]carbamoyl]-4-methoxy-3-pyridyl]oxymethyl 2-methylpropanoate, [4-methoxy-2-[[(1S)-1-methyl-2-[(1S)-1-[1-(1-naphthyl)cyclopropyl]ethoxy]-2-oxo-ethyl]carbamoyl]-3-pyridyl]oxymethyl 2-methylpropanoate; [2-(4-bromophenyl)-1,2-dimethyl-propyl](2S)-2-[(3-hydroxy-4-methoxy-pyridine-2-carbonyl)amino]propanoate; 1-(1-phenyl cyclohexyl)ethyl (2S)-2-[(3-hydroxy-4-methoxy-pyridine-2-carbonyl)amino]propanoate; [1-methyl-2-(2-quinolyl)propyl](2S)-2-[(3-hydroxy-4-methoxy-pyridine-2-carbonyl)amino]propanoate; [2-(7-bromoindol-1-yl)-1-methyl-propyl](2S)-2-[(3-hydroxy-4-methoxy-pyridine-2-carbonyl)amino]propanoate; [1-methyl-2-[6-(trifluoromethyl)indol-1-yl]propyl](2S)-2-[(3-hydroxy-4-methoxy-pyridine-2-carbonyl)amino]propanoate, (2-indazol-1-yl-1-methyl-propyl) (2S)-2-[(3-hydroxy-4-methoxy-pyridine-2-carbonyl)amino]propanoate, [2-(5-chloro-2-thienyl)-1-methyl-propyl](2S)-2-[(3-hydroxy-4-methoxy-pyridine-2-carbonyl)amino]propanoate, [2-(4,7-dichloroindol-1-yl)-1-methyl-propyl](2S)-2-[(3-acetoxy-4-methoxy-pyridine-2-carbonyl)amino]propanoate, [2-(7-bromo-4-fluoro-indol-1-yl)-1-methyl-propyl](2S)-2-[(3-acetoxy-4-methoxy-pyridine-2-carbonyl)amino]propanoate, [(1S)-1-[1-(1-naphthyl)cyclopropyl]ethyl](2S)-2-[(3-acetoxy-4-methoxy-pyridine-2-carbonyl)amino]propanoate (these compounds may be prepared from the methods described in WO2020/208096); methyl N-[[5-[1-(2,6-difluoro-4-isopropyl-phenyl)pyrazol-3-yl]-2-methyl-phenyl]methyl]carbamate; methyl N-[[5-[1-(4-cyclopropyl-2,6-difluoro-phenyl)pyrazol-3-yl]-2-methyl-phenyl]methyl]carbamate; (2E)-2-methoxyimino-N-methyl-2-[3-methyl-2-[[(E)-1-[4-(trifluoromethyl)-2-pyridyl]ethylideneamino]oxymethyl]phenyl]acetamide; 6-chloro-3-(3-cyclopropyl-2-fluoro-phenoxy)-N-[2-(2,4-dimethylphenyl)-2,2-difluoro-ethyl]-5-methyl-pyridazine-4-carboxamide; 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide; 6-chloro-3-(3-cyclopropyl-2-fluoro-phenoxy)-N-[2-(3,4-dimethylphenyl)-2,2-difluoro-ethyl]-5-methyl-pyridazine-4-carboxamide; 5-[5-(difluoromethyl)-1,3,4-oxadiazol-2-yl]-N-[1-(2,6-difluorophenyl)ethyl]pyrimidin-2-amine; 2-(difluoromethyl)-5-[2-[1-(2,6-difluorophenyl)cyclopropoxy]pyrimidin-5-yl]-1,3,4-oxadiazole; 5-[5-(difluoromethyl)-1,3,4-oxadiazol-2-yl]-N-[1-(2,6-difluorophenyl)cyclopropyl]pyrimidin-2-amine; 5-[5-(difluoromethyl)-1,3,4-oxadiazol-2-yl]-N-[1-(2-fluorophenyl)cyclopropyl]pyrimidin-2-amine; N-[1-(2-fluorophenyl)cyclopropyl]-5-[5-(trifluoromethyl)-1,3,4-oxadiazol-2-yl]pyrimidin-2-amine; 5-[5-(difluoromethyl)-1,3,4-oxadiazol-2-yl]-N-[1-(2-fluorophenyl)ethyl]pyrimidin-2-amine; 5-[5-(difluoromethyl)-1,3,4-oxadiazol-2-yl]-N-[1-(3,5-difluorophenyl)ethyl]pyrimidin-2-amine; ethyl 1-[[4-[[2-(trifluoromethyl)-1,3-dioxolan-2-yl]methoxy]phenyl]methyl]-1H-pyrazole-4-carboxylate; ethyl 1-[[4-[[(1Z)-2-ethoxy-3,3,3-trifluoro-1-propen-1-yl]oxy]phenyl]methyl]-1H-pyrazole-4-carboxylate; 3-[[3-chloro-2-(2-ethylpyrazol-3-yl)phenyl]methyl]-7,8-difluoro-2-methyl-quinoline; N-[2-(2-ethylpyrazol-3-yl)phenyl]-5,6-difluoro-3-methyl-quinoxalin-2-amine; 5,6-difluoro-N-[3-fluoro-2-(2-propylpyrazol-3-yl)phenyl]-3-methyl-quinoxalin-2-amine; N-[3-chloro-2-(2-propylpyrazol-3-yl)phenyl]-7,8-difluoro-2-methyl-quinolin-3-amine; 1-(5,6-dimethyl-3-pyridyl)-4,4-difluoro-3,3-dimethyl-isoquinoline; 1-[6-(difluoromethyl)-5-methyl-3-pyridyl]-4,4-difluoro-3,3-dimethyl-isoquinoline; Methyl 2-[2-chloro-4-(4-chlorophenoxy)phenyl]-2-hydroxy-3-(1,2,4-triazol-1-yl)propanoate and 2-(1-chlorocyclopropyl)-1-(2-chlorophenyl)-3-(1,2,4-triazol-1-yl)propan-2-ol.

In a preferred embodiment suitable additional active ingredients are selected from the group consisting of benzovindiflupyr, fluxapyroxad, pydiflumetofen, isopyrazam, fluopyram, penthiopyrad, sedaxane, bixafen, difenoconazole, cyproconazole, tebuconazole, hexaconazole, prothioconazole, propiconazole, epoxiconazole, metconazole, tetraconazole, fluoxytioconazole, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentanecarboxylate, flutriafol, mefentrifluconazole, ipconazole, paclobutrazol, azoxystrobin, trifloxystrobin, picoxystrobin, pyraclostrobin, metalaxyl-M, fenpropidin, fenpropimorph, cyprodinil, spiroxamine, mancozeb, chlorothalonil, folpet, copper-oxychloride, copper-hydroxide, Sulphur, oxathiapiprolin, ipflufenoquin, quinofumelin, mandipropamid, fluazinam, fludioxinil, fosetyl-aluminium, acibenzolar-Smethyl, enestrobin, inpyrfluxam, fluindapyr, isoflucypram, metyltetraprole, florylpicoxamid, metarylpicoxamide, flefunoxadiazam isofetamid, procymidone, carbendazim, fenhexamid, prochloraz, prohexadione-calcium, melaluca alternifolia oil (an extract of the tea tree plant Melaluca alternifolia (commercially available as Timorex Gold®, which is a broad-spectrum botanical biofungicide)), N′-[5-bromo-2-methyl-6-(1-methyl-2-propoxy-ethoxy)-3-pyridyl]-N-ethyl-N-methylformamidine, cyclobutrifluram calcium phosphonate, cis-jasmone, trinexapac-ethyl, glyphosate, 2,4-D (2,4-dichlorophenoxyacetic acid) and thiamethoxam.

The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The polymorphs of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The polymorphs of the invention may be used in combination with derivatives and analogues of the general class of dioxomorpholine antiparasitic agents as described in WO9615121 and also with anthelmintic active cyclic depsipeptides such as those described in WO9611945, WO9319053, WO 9325543, EP-626375, EP-382173, WO-9419334, EP-382173, and EP-503538.

The polymorphs of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The polymorphs of the invention may be used in combination with terpene alkaloids, for example those described in WO95/19363 or WO04/72086, particularly the compounds disclosed therein.

Other examples of such biologically active compounds that the compounds of the invention may be used in combination with include but are not restricted to the following: Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195,RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus, and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The following mixtures of the polymorphs with active ingredients are preferred:
a compound selected from the group of substances consisting of petroleum oils+((I-A) or (I-B)), 1,1-bis(4-chlorophenyl)-2-ethoxyethanol+((I-A) or (I-B)), 2,4-dichlorophenyl benzenesulfonate+((I-A) or (I-B)), 2-fluoro-N-methyl-N-1-naphthylacetamide+((I-A) or (I-B)), 4-chlorophenyl phenyl sulfone+((I-A) or (I-B)), acetoprole+((I-A) or (I-B)), aldoxycarb+((I-A) or (I-B)), amidithion+((I-A) or (I-B)), amidothioate+((I-A) or (I-B)), amiton+((I-A) or (I-B)), amiton hydrogen oxalate+((I-A) or (I-B)), amitraz+((I-A) or (I-B)), aramite+((I-A) or (I-B)), arsenous oxide+((I-A) or (I-B)), azobenzene+((I-A) or (I-B)), azothoate+((I-A) or (I-B)), benomyl+((I-A) or (I-B)), benoxafos+((I-A) or (I-B)), benzyl benzoate+((I-A) or (I-B)), bixafen+((I-A) or (I-B)), brofenvalerate+((I-A) or (I-B)), bromocyclen+((I-A) or (I-B)), bromophos+((I-A) or (I-B)), bromopropylate+((I-A) or (I-B)), buprofezin+((I-A) or (I-B)), butocarboxim+((I-A) or (I-B)), butoxycarboxim+((I-A) or (I-B)), butylpyridaben+((I-A) or (I-B)), calcium polysulfide+((I-A) or (I-B)), camphechlor+((I-A) or (I-B)), carbanolate+((I-A) or (I-B)), carbophenothion+((I-A) or (I-B)), cymiazole+((I-A) or (I-B)), chino-methionat+((I-A) or (I-B)), chlorbenside+((I-A) or (I-B)), chlordimeform+((I-A) or (I-B)), chlordimeform hydrochloride+((I-A) or (I-B)), chlorfenethol+((I-A) or (I-B)), chlorfenson+((I-A) or (I-B)), chlorfensulfide+((I-A) or (I-B)), chlorobenzilate+((I-A) or (I-B)), chloromebuform+((I-A) or (I-B)), chloromethiuron+((I-A) or (I-B)), chloropropylate+((I-A) or (I-B)), chlorthiophos+((I-A) or (I-B)), cinerin I+((I-A) or (I-B)), cinerin II+((I-A) or (I-B)), cinerins+((I-A) or (I-B)), closantel+((I-A) or (I-B)), coumaphos+((I-A) or (I-B)), crotamiton+((I-A) or (I-B)), crotoxyphos+((I-A) or (I-B)), cufraneb+((I-A) or (I-B)), cyanthoate+((I-A) or (I-B)), DCPM+((I-A) or (I-B)), DDT+((I-A) or (I-B)), demephion+((I-A) or (I-B)), demephion-O+((I-A) or (I-B)), demephion-S+((I-A) or (I-B)), demeton-methyl+((I-A) or (I-B)), demeton-O+((I-A) or (I-B)), demeton-O-methyl+((I-A) or (I-B)), demeton-S+((I-A) or (I-B)), demeton-S-methyl+((I-A) or (I-B)), demeton-S-methylsulfon+((I-A) or (I-B)), dichlofluanid+((I-A) or (I-B)), dichlorvos+((I-A) or (I-B)), dicliphos+((I-A) or (I-B)), dienochlor+((I-A) or (I-B)), dimefox+((I-A) or (I-B)), dinex+((I-A) or (I-B)), dinex-diclexine+((I-A) or (I-B)), dinocap-4+((I-A) or (I-B)), dinocap-6+((I-A) or (I-B)), dinocton+((I-A) or (I-B)), dinopenton+((I-A) or (I-B)), dinosulfon+((I-A) or (I-B)), dinoterbon+((I-A) or (I-B)), dioxathion+((I-A) or (I-B)), diphenyl sulfone+((I-A) or (I-B)), disulfiram+((I-A) or (I-B)), DNOC+((I-A) or (I-B)), dofenapyn+((I-A) or (I-B)), doramectin+((I-A) or (I-B)), endothion+((I-A) or (I-B)), eprinomectin+((I-A) or (I-B)), ethoate-methyl+((I-A) or (I-B)), etrimfos+((I-A) or (I-B)), fenazaflor+((I-A) or (I-B)), fenbutatin oxide+((I-A) or (I-B)), fenothiocarb+((I-A) or (I-B)), fenpyrad+((I-A) or (I-B)), fenpyroximate+((I-A) or (I-B)), fenpyrazamine+((I-A) or (I-B)), fenson+((I-A) or (I-B)), fentrifanil+((I-A) or (I-B)), flubenzimine+((I-A) or (I-B)), flucycloxuron+((I-A) or (I-B)), fluenetil+((I-A) or (I-B)), fluorbenside+((I-A) or (I-B)), FMC 1137+((I-A) or (I-B)), formetanate+((I-A) or (I-B)), formetanate hydrochloride+((I-A) or (I-B)), formparanate+((I-A) or (I-B)), gamma-HCH+((I-A) or (I-B)), glyodin+((I-A) or (I-B)), halfenprox+((I-A) or (I-B)), hexadecyl cyclopropanecarboxylate+((I-A) or (I-B)), isocarbophos+((I-A) or (I-B)), jasmolin I+((I-A) or (I-B)), jasmolin II+((I-A) or (I-B)), jodfenphos+((I-A) or (I-B)), lindane+((I-A) or (I-B)), malonoben+((I-A) or (I-B)), mecarbam+((I-A) or (I-B)), mephosfolan+((I-A) or (I-B)), mesulfen+((I-A) or (I-B)), methacrifos+((I-A) or (I-B)), methyl bromide+((I-A) or (I-B)), metolcarb+((I-A) or (I-B)), mexacarbate+((I-A) or (I-B)), milbemycin oxime+((I-A) or (I-B)), mipafox+((I-A) or (I-B)), monocrotophos+((I-A) or (I-B)), morphothion+((I-A) or (I-B)), moxidectin+((I-A) or (I-B)), naled+((I-A) or (I-B)), 4-chloro-2-(2-chloro-2-methyl-propyl)-5-[(6-iodo-3-pyridyl)methoxy]pyridazin-3-one+((I-A) or (I-B)), nifluridide+((I-A) or (I-B)), nikkomycins+((I-A) or (I-B)), nitrilacarb+((I-A) or (I-B)), nitrilacarb 1:1 zinc chloride complex+((I-A) or (I-B)), omethoate+((I-A) or (I-B)), oxydeprofos+((I-A) or (I-B)), oxydisulfoton+((I-A) or (I-B)), pp′-DDT+((I-A) or (I-B)), parathion+((I-A) or (I-B)), permethrin+((I-A) or (I-B)), phenkapton+((I-A) or (I-B)), phosalone+((I-A) or (I-B)), phosfolan+((I-A) or (I-B)), phosphamidon+((I-A) or (I-B)), polychloroterpenes+((I-A) or (I-B)), polynactins+((I-A) or (I-B)), proclonol+((I-A) or (I-B)), promacyl+((I-A) or (I-B)), propoxur+((I-A) or (I-B)), prothidathion+((I-A) or (I-B)), prothoate+((I-A) or (I-B)), pyrethrin I+((I-A) or (I-B)), pyrethrin II+((I-A) or (I-B)), pyrethrins+((I-A) or (I-B)), pyridaphenthion+((I-A) or (I-B)), pyrimitate+((I-A) or (I-B)), quinalphos+((I-A) or (I-B)), quintiofos+((I-A) or (I-B)), R-1492+((I-A) or (I-B)), phosglycin+((I-A) or (I-B)), rotenone+((I-A) or (I-B)), schradan+((I-A) or (I-B)), sebufos+((I-A) or (I-B)), selamectin+((I-A) or (I-B)), sophamide+((I-A) or (I-B)), SSI-121+((I-A) or (I-B)), sulfiram+((I-A) or (I-B)), sulfluramid+((I-A) or (I-B)), sulfotep+((I-A) or (I-B)), sulfur+((I-A) or (I-B)), diflovidazin+((I-A) or (I-B)), tau-fluvalinate+((I-A) or (I-B)), TEPP+((I-A) or (I-B)), terbam+((I-A) or (I-B)), tetradifon+((I-A) or (I-B)), tetrasul+((I-A) or (I-B)), thiafenox+((I-A) or (I-B)), thiocarboxime+((I-A) or (I-B)), thiofanox+((I-A) or (I-B)), thiometon+((I-A) or (I-B)), thioquinox+((I-A) or (I-B)), thuringiensin+((I-A) or (I-B)), triamiphos+((I-A) or (I-B)), triarathene+((I-A) or (I-B)), triazophos+((I-A) or (I-B)), triazuron+((I-A) or (I-B)), trifenofos+((I-A) or (I-B)), trinactin+((I-A) or (I-B)), vamidothion+((I-A) or (I-B)), vaniliprole+((I-A) or (I-B)), bethoxazin+((I-A) or (I-B)), copper dioctanoate+((I-A) or (I-B)), copper sulfate+((I-A) or (I-B)), cybutryne+((I-A) or (I-B)), dichlone+((I-A) or (I-B)), dichlorophen+((I-A) or (I-B)), endothal+((I-A) or (I-B)), fentin+((I-A) or (I-B)), hydrated lime+((I-A) or (I-B)), nabam+((I-A) or (I-B)), quinoclamine+((I-A) or (I-B)), quinonamid+((I-A) or (I-B)), simazine+((I-A) or (I-B)), triphenyltin acetate+((I-A) or (I-B)), triphenyltin hydroxide+((I-A) or (I-B)), crufomate+((I-A) or (I-B)), piperazine+((I-A) or (I-B)), thiophanate+((I-A) or (I-B)), chloralose+((I-A) or (I-B)), fenthion+((I-A) or (I-B)), pyridin-4-amine+((I-A) or (I-B)), strychnine+((I-A) or (I-B)), 1-hydroxy-1H-pyridine-2-thione+((I-A) or (I-B)), 4-(quinoxalin-2-ylamino)benzenesulfonamide+((I-A) or (I-B)), 8-hydroxyquinoline sulfate+((I-A) or (I-B)), bronopol+((I-A) or (I-B)), copper hydroxide+((I-A) or (I-B)), cresol+((I-A) or (I-B)), dipyrithione+((I-A) or (I-B)), dodicin+((I-A) or (I-B)), fenaminosulf+((I-A) or (I-B)), formaldehyde+((I-A) or (I-B)), hydrargaphen+((I-A) or (I-B)), kasugamycin+((I-A) or (I-B)), kasugamycin hydrochloride hydrate+((I-A) or (I-B)), nickel bis(dimethyldithiocarbamate)+((I-A) or (I-B)), nitrapyrin+((I-A) or (I-B)), octhilinone+((I-A) or (I-B)), oxolinic acid+((I-A) or (I-B)), oxytetracycline+((I-A) or (I-B)), potassium hydroxyquinoline sulfate+((I-A) or (I-B)), probenazole+((I-A) or (I-B)), streptomycin+((I-A) or (I-B)), streptomycin sesquisulfate+((I-A) or (I-B)), tecloftalam+((I-A) or (I-B)), thiomersal+((I-A) or (I-B)), Adoxophyes orana GV+((I-A) or (I-B)), Agrobacterium radiobacter+((I-A) or (I-B)), Amblyseius spp.+((I-A) or (I-B)), Anagrapha falcifera NPV+((I-A) or (I-B)), Anagrus atomus+((I-A) or (I-B)), Aphelinus abdominalis+((I-A) or (I-B)), Aphidius colemani+((I-A) or (I-B)), Aphidoletes aphidimyza+((I-A) or (I-B)), Autographa californica NPV+((I-A) or (I-B)), Bacillus sphaericus Neide+((I-A) or (I-B)), Beauveria brongniartii+((I-A) or (I-B)), Chrysoperla carnea+((I-A) or (I-B)), Cryptolaemus montrouzieri+((I-A) or (I-B)), Cydia pomonella GV+((I-A) or (I-B)), Dacnusa sibirica+((I-A) or (I-B)), Diglyphus isaea+((I-A) or (I-B)), Encarsia formosa+((I-A) or (I-B)), Eretmocerus eremicus+((I-A) or (I-B)), Heterorhabditis bacteriophora and H. megidis+((I-A) or (I-B)), Hippodamia convergens+((I-A) or (I-B)), Leptomastix dactylopii+((I-A) or (I-B)), Macrolophus caliginosus+((I-A) or (I-B)), Mamestra brassicae NPV+((I-A) or (I-B)), Metaphycus helvolus+((I-A) or (I-B)), Metarhizium anisopliae var. acridum+((I-A) or (I-B)), Metarhizium anisopliae var. anisopliae+((I-A) or (I-B)), Neodiprion sertifer NPV and N. lecontei NPV+((I-A) or (I-B)), Orius spp.+((I-A) or (I-B)), Paecilomyces fumosoroseus+((I-A) or (I-B)), Phytoseiulus persimilis+((I-A) or (I-B)), Steinernema bibionis+((I-A) or (I-B)), Steinernema carpocapsae+((I-A) or (I-B)), Steinernema feltiae+((I-A) or (I-B)), Steinernema glaseri+((I-A) or (I-B)), Steinernema riobrave+((I-A) or (I-B)), Steinernema riobravis+((I-A) or (I-B)), Steinernema scapterisci+((I-A) or (I-B)), Steinernema spp.+((I-A) or (I-B)), Trichogramma spp.+((I-A) or (I-B)), Typhlodromus occidentalis+((I-A) or (I-B)), Verticillium lecanii+((I-A) or (I-B)), apholate+((I-A) or (I-B)), bisazir+((I-A) or (I-B)), busulfan+((I-A) or (I-B)), dimatif+((I-A) or (I-B)), hemel+((I-A) or (I-B)), hempa+((I-A) or (I-B)), metepa+((I-A) or (I-B)), methiotepa+((I-A) or (I-B)), methyl apholate+((I-A) or (I-B)), morzid+((I-A) or (I-B)), penfluron+((I-A) or (I-B)), tepa+((I-A) or (I-B)), thiohempa+((I-A) or (I-B)), thiotepa+((I-A) or (I-B)), tretamine+((I-A) or (I-B)), uredepa+((I-A) or (I-B)), (E)-dec-5-en-1-yl acetate with (E)-dec-5-en-1-ol+((I-A) or (I-B)), (E)-tridec-4-en-1-yl acetate+((I-A) or (I-B)), (E)-6-methylhept-2-en-4-ol+((I-A) or (I-B)), (E,Z)-tetradeca-4,10-dien-1-yl acetate+((I-A) or (I-B)), (Z)-dodec-7-en-1-yl acetate+((I-A) or (I-B)), (Z)-hexadec-11-enal+((I-A) or (I-B)), (Z)-hexadec-11-en-1-yl acetate+((I-A) or (I-B)), (Z)-hexadec-13-en-11-yn-1-yl acetate+((I-A) or (I-B)), (Z)-icos-13-en-10-one+((I-A) or (I-B)), (Z)-tetradec-7-en-1-al+((I-A) or (I-B)), (Z)-tetradec-9-en-1-ol+((I-A) or (I-B)), (Z)-tetradec-9-en-1-yl acetate+((I-A) or (I-B)), (7E,9Z)-dodeca-7,9-dien-1-yl acetate+((I-A) or (I-B)), (9Z,11E)-tetradeca-9,11-dien-1-yl acetate+((I-A) or (I-B)), (9Z,12E)-tetradeca-9,12-dien-1-yl acetate+((I-A) or (I-B)), 14-methyloctadec-1-ene+((I-A) or (I-B)), 4-methylnonan-5-ol with 4-methylnonan-5-one+((I-A) or (I-B)), alpha-multistriatin+((I-A) or (I-B)), brevicomin+((I-A) or (I-B)), codlelure+((I-A) or (I-B)), codlemone+((I-A) or (I-B)), cuelure+((I-A) or (I-B)), disparlure+((I-A) or (I-B)), dodec-8-en-1-yl acetate+((I-A) or (I-B)), dodec-9-en-1-yl acetate+((I-A) or (I-B)), dodeca-8+((I-A) or (I-B)), 10-dien-1-yl acetate+((I-A) or (I-B)), dominicalure+((I-A) or (I-B)), ethyl 4-methyloctanoate+((I-A) or (I-B)), eugenol+((I-A) or (I-B)), frontalin+((I-A) or (I-B)), grandlure+((I-A) or (I-B)), grandlure I+((I-A) or (I-B)), grandlure II+((I-A) or (I-B)), grandlure III+((I-A) or (I-B)), grandlure IV+((I-A) or (I-B)), hexalure+((I-A) or (I-B)), ipsdienol+((I-A) or (I-B)), ipsenol+((I-A) or (I-B)), japonilure+((I-A) or (I-B)), lineatin+((I-A) or (I-B)), litlure+((I-A) or (I-B)), looplure+((I-A) or (I-B)), medlure+((I-A) or (I-B)), megatomoic acid+((I-A) or (I-B)), methyl eugenol+((I-A) or (I-B)), muscalure+((I-A) or (I-B)), octadeca-2,13-dien-1-yl acetate+((I-A) or (I-B)), octadeca-3,13-dien-1-yl acetate+((I-A) or (I-B)), orfralure+((I-A) or (I-B)), oryctalure+((I-A) or (I-B)), ostramone+((I-A) or (I-B)), siglure+((I-A) or (I-B)), sordidin+((I-A) or (I-B)), sulcatol+((I-A) or (I-B)), tetradec-11-en-1-yl acetate+((I-A) or (I-B)), trimedlure+((I-A) or (I-B)), trimedlure A+((I-A) or (I-B)), trimedlure B₁+((I-A) or (I-B)), trimedlure B₂+((I-A) or (I-B)), trimedlure C+((I-A) or (I-B)), trunc-call+((I-A) or (I-B)), 2-(octylthio)ethanol+((I-A) or (I-B)), butopyronoxyl+((I-A) or (I-B)), butoxy(polypropylene glycol)+((I-A) or (I-B)), dibutyl adipate+((I-A) or (I-B)), dibutyl phthalate+((I-A) or (I-B)), dibutyl succinate+((I-A) or (I-B)), diethyltoluamide+((I-A) or (I-B)), dimethyl carbate+((I-A) or (I-B)), dimethyl phthalate+((I-A) or (I-B)), ethyl hexanediol+((I-A) or (I-B)), hexamide+((I-A) or (I-B)), methoquin-butyl+((I-A) or (I-B)), methylneodecanamide+((I-A) or (I-B)), oxamate+((I-A) or (I-B)), picaridin+((I-A) or (I-B)), 1-dichloro-1-nitroethane+((I-A) or (I-B)), 1,1-dichloro-2,2-bis(4-ethylphenyl)ethane+((I-A) or (I-B)), 1,2-dichloropropane with 1,3-dichloropropene+((I-A) or (I-B)), 1-bromo-2-chloroethane+((I-A) or (I-B)), 2,2,2-trichloro-1-(3,4-dichlorophenyl)ethyl acetate+((I-A) or (I-B)), 2,2-dichlorovinyl 2-ethylsulfinylethyl methyl phosphate+((I-A) or (I-B)), 2-(1,3-dithiolan-2-yl)phenyl dimethylcarbamate+((I-A) or (I-B)), 2-(2-butoxyethoxy)ethyl thiocyanate+((I-A) or (I-B)), 2-(4,5-dimethyl-1,3-dioxolan-2-yl)phenyl methylcarbamate+((I-A) or (I-B)), 2-(4-chloro-3,5-xylyloxy)ethanol+((I-A) or (I-B)), 2-chlorovinyl diethyl phosphate+((I-A) or (I-B)), 2-imidazolidone+((I-A) or (I-B)), 2-isovalerylindan-1,3-dione+((I-A) or (I-B)), 2-methyl(prop-2-ynyl)aminophenyl methylcarbamate+((I-A) or (I-B)), 2-thiocyanatoethyl laurate+((I-A) or (I-B)), 3-bromo-1-chloroprop-1-ene+((I-A) or (I-B)), 3-methyl-1-phenylpyrazol-5-yl dimethylcarbamate+((I-A) or (I-B)), 4-methyl(prop-2-ynyl)amino-3,5-xylyl methylcarbamate+((I-A) or (I-B)), 5,5-dimethyl-3-oxocyclohex-1-enyl dimethylcarbamate+((I-A) or (I-B)), acethion+((I-A) or (I-B)), acrylonitrile+((I-A) or (I-B)), aldrin+((I-A) or (I-B)), allosamidin+((I-A) or (I-B)), allyxycarb+((I-A) or (I-B)), alpha-ecdysone+((I-A) or (I-B)), aluminium phosphide+((I-A) or (I-B)), aminocarb+((I-A) or (I-B)), anabasine+((I-A) or (I-B)), athidathion+((I-A) or (I-B)), azamethiphos+((I-A) or (I-B)), Bacillus thuringiensis delta endotoxins+((I-A) or (I-B)), barium hexafluorosilicate+((I-A) or (I-B)), barium polysulfide+((I-A) or (I-B)), barthrin+((I-A) or (I-B)), Bayer 22/190+((I-A) or (I-B)), Bayer 22408+((I-A) or (I-B)), beta-cyfluthrin+((I-A) or (I-B)), beta-cypermethrin+((I-A) or (I-B)), bioethanomethrin+((I-A) or (I-B)), biopermethrin+((I-A) or (I-B)), bis(2-chloroethyl) ether+((I-A) or (I-B)), borax+((I-A) or (I-B)), bromfenvinfos+((I-A) or (I-B)), bromo-DDT+((I-A) or (I-B)), bufencarb+((I-A) or (I-B)), butacarb+((I-A) or (I-B)), butathiofos+((I-A) or (I-B)), butonate+((I-A) or (I-B)), calcium arsenate+((I-A) or (I-B)), calcium cyanide+((I-A) or (I-B)), carbon disulfide+((I-A) or (I-B)), carbon tetrachloride+((I-A) or (I-B)), cartap hydrochloride+((I-A) or (I-B)), cevadine+((I-A) or (I-B)), chlorbicyclen+((I-A) or (I-B)), chlordane+((I-A) or (I-B)), chlordecone+((I-A) or (I-B)), chloroform+((I-A) or (I-B)), chloropicrin+((I-A) or (I-B)), chlorphoxim+((I-A) or (I-B)), chlorprazophos+((I-A) or (I-B)), cis-resmethrin+((I-A) or (I-B)), cismethrin+((I-A) or (I-B)), clocythrin+((I-A) or (I-B)), copper acetoarsenite+((I-A) or (I-B)), copper arsenate+((I-A) or (I-B)), copper oleate+((I-A) or (I-B)), coumithoate+((I-A) or (I-B)), cryolite+((I-A) or (I-B)), CS 708+((I-A) or (I-B)), cyanofenphos+((I-A) or (I-B)), cyanophos+((I-A) or (I-B)), cyclethrin+((I-A) or (I-B)), cythioate+((I-A) or (I-B)), d-tetramethrin+((I-A) or (I-B)), DAEP+((I-A) or (I-B)), dazomet+((I-A) or (I-B)), decarbofuran+((I-A) or (I-B)), diamidafos+((I-A) or (I-B)), dicapthon+((I-A) or (I-B)), dichlofenthion+((I-A) or (I-B)), dicresyl+((I-A) or (I-B)), dicyclanil+((I-A) or (I-B)), dieldrin+((I-A) or (I-B)), diethyl 5-methylpyrazol-3-yl phosphate+((I-A) or (I-B)), dilor+((I-A) or (I-B)), dimefluthrin+((I-A) or (I-B)), dimetan+((I-A) or (I-B)), dimethrin+((I-A) or (I-B)), dimethylvinphos+((I-A) or (I-B)), dimetilan+((I-A) or (I-B)), dinoprop+((I-A) or (I-B)), dinosam+((I-A) or (I-B)), dinoseb+((I-A) or (I-B)), diofenolan+((I-A) or (I-B)), dioxabenzofos+((I-A) or (I-B)), dithicrofos+((I-A) or (I-B)), DSP+((I-A) or (I-B)), ecdysterone+((I-A) or (I-B)), EI 1642+((I-A) or (I-B)), EMPC+((I-A) or (I-B)), EPBP+((I-A) or (I-B)), etaphos+((I-A) or (I-B)), ethiofencarb+((I-A) or (I-B)), ethyl formate+((I-A) or (I-B)), ethylene dibromide+((I-A) or (I-B)), ethylene dichloride+((I-A) or (I-B)), ethylene oxide+((I-A) or (I-B)), EXD+((I-A) or (I-B)), fenchlorphos+((I-A) or (I-B)), fenethacarb+((I-A) or (I-B)), fenitrothion+((I-A) or (I-B)), fenoxacrim+((I-A) or (I-B)), fenpirithrin+((I-A) or (I-B)), fensulfothion+((I-A) or (I-B)), fenthion-ethyl+((I-A) or (I-B)), flucofuron+((I-A) or (I-B)), fosmethilan+((I-A) or (I-B)), fospirate+((I-A) or (I-B)), fosthietan+((I-A) or (I-B)), furathiocarb+((I-A) or (I-B)), furethrin+((I-A) or (I-B)), guazatine+((I-A) or (I-B)), guazatine acetates+((I-A) or (I-B)), sodium tetrathiocarbonate+((I-A) or (I-B)), halfenprox+((I-A) or (I-B)), HCH+((I-A) or (I-B)), HEOD+((I-A) or (I-B)), heptachlor+((I-A) or (I-B)), heterophos+((I-A) or (I-B)), HHDN+((I-A) or (I-B)), hydrogen cyanide+((I-A) or (I-B)), hyquincarb+((I-A) or (I-B)), IPSP+((I-A) or (I-B)), isazofos+((I-A) or (I-B)), isobenzan+((I-A) or (I-B)), isodrin+((I-A) or (I-B)), isofenphos+((I-A) or (I-B)), isolane+((I-A) or (I-B)), isoprothiolane+((I-A) or (I-B)), isoxathion+((I-A) or (I-B)), juvenile hormone I+((I-A) or (I-B)), juvenile hormone II+((I-A) or (I-B)), juvenile hormone III+((I-A) or (I-B)), kelevan+((I-A) or (I-B)), kinoprene+((I-A) or (I-B)), lead arsenate+((I-A) or (I-B)), leptophos+((I-A) or (I-B)), lirimfos+((I-A) or (I-B)), lythidathion+((I-A) or (I-B)), m-cumenyl methylcarbamate+((I-A) or (I-B)), magnesium phosphide+((I-A) or (I-B)), mazidox+((I-A) or (I-B)), mecarphon+((I-A) or (I-B)), menazon+((I-A) or (I-B)), mercurous chloride+((I-A) or (I-B)), mesulfenfos+((I-A) or (I-B)), metam+((I-A) or (I-B)), metam-potassium+((I-A) or (I-B)), metam-sodium+((I-A) or (I-B)), methanesulfonyl fluoride+((I-A) or (I-B)), methocrotophos+((I-A) or (I-B)), methoprene+((I-A) or (I-B)), methothrin+((I-A) or (I-B)), methoxychlor+((I-A) or (I-B)), methyl isothiocyanate+((I-A) or (I-B)), methylchloroform+((I-A) or (I-B)), methylene chloride+((I-A) or (I-B)), metoxadiazone+((I-A) or (I-B)), mirex+((I-A) or (I-B)), naftalofos+((I-A) or (I-B)), naphthalene+((I-A) or (I-B)), NC-170+((I-A) or (I-B)), nicotine+((I-A) or (I-B)), nicotine sulfate+((I-A) or (I-B)), nithiazine+((I-A) or (I-B)), nornicotine+((I-A) or (I-B)), 0-5-dichloro-4-iodophenyl O-ethyl ethylphosphonothioate+((I-A) or (I-B)), O,O-diethyl O-4-methyl-2-oxo-2H-chromen-7-yl phosphorothioate+((I-A) or (I-B)), O,O-diethyl O-6-methyl-2-propylpyrimidin-4-yl phosphorothioate+((I-A) or (I-B)), O,O,O′,O′-tetrapropyl dithiopyrophosphate+((I-A) or (I-B)), oleic acid+((I-A) or (I-B)), para-dichlorobenzene+((I-A) or (I-B)), parathion-methyl+((I-A) or (I-B)), pentachlorophenol+((I-A) or (I-B)), pentachlorophenyl laurate+((I-A) or (I-B)), PH 60-38+((I-A) or (I-B)), phenkapton+((I-A) or (I-B)), phosnichlor+((I-A) or (I-B)), phosphine+((I-A) or (I-B)), phoxim-methyl+((I-A) or (I-B)), pirimetaphos+((I-A) or (I-B)), polychlorodicyclopentadiene isomers+((I-A) or (I-B)), potassium arsenite+((I-A) or (I-B)), potassium thiocyanate+((I-A) or (I-B)), precocene I+((I-A) or (I-B)), precocene II+((I-A) or (I-B)), precocene III+((I-A) or (I-B)), primidophos+((I-A) or (I-B)), profluthrin+((I-A) or (I-B)), promecarb+((I-A) or (I-B)), prothiofos+((I-A) or (I-B)), pyrazophos+((I-A) or (I-B)), pyresmethrin+((I-A) or (I-B)), quassia+((I-A) or (I-B)), quinalphos-methyl+((I-A) or (I-B)), quinothion+((I-A) or (I-B)), rafoxanide+((I-A) or (I-B)), resmethrin+((I-A) or (I-B)), rotenone+((I-A) or (I-B)), kadethrin+((I-A) or (I-B)), ryania+((I-A) or (I-B)), ryanodine+((I-A) or (I-B)), sabadilla)+((I-A) or (I-B)), schradan+((I-A) or (I-B)), sebufos+((I-A) or (I-B)), SI-0009+((I-A) or (I-B)), thiapronil+((I-A) or (I-B)), sodium arsenite+((I-A) or (I-B)), sodium cyanide+((I-A) or (I-B)), sodium fluoride+((I-A) or (I-B)), sodium hexafluorosilicate+((I-A) or (I-B)), sodium pentachlorophenoxide+((I-A) or (I-B)), sodium selenate+((I-A) or (I-B)), sodium thiocyanate+((I-A) or (I-B)), sulcofuron+((I-A) or (I-B)), sulcofuron-sodium+((I-A) or (I-B)), sulfuryl fluoride+((I-A) or (I-B)), sulprofos+((I-A) or (I-B)), tar oils+((I-A) or (I-B)), tazimcarb+((I-A) or (I-B)), TDE+((I-A) or (I-B)), tebupirimfos+((I-A) or (I-B)), temephos+((I-A) or (I-B)), terallethrin+((I-A) or (I-B)), tetrachloroethane+((I-A) or (I-B)), thicrofos+((I-A) or (I-B)), thiocyclam+((I-A) or (I-B)), thiocyclam hydrogen oxalate+((I-A) or (I-B)), thionazin+((I-A) or (I-B)), thiosultap+((I-A) or (I-B)), thiosultap-sodium+((I-A) or (I-B)), tralomethrin+((I-A) or (I-B)), transpermethrin+((I-A) or (I-B)), triazamate+((I-A) or (I-B)), trichlormetaphos-3+((I-A) or (I-B)), trichloronat+((I-A) or (I-B)), trimethacarb+((I-A) or (I-B)), tolprocarb+((I-A) or (I-B)), triclopyricarb+((I-A) or (I-B)), triprene+((I-A) or (I-B)), veratridine+((I-A) or (I-B)), veratrine+((I-A) or (I-B)), XMC+((I-A) or (I-B)), zetamethrin+((I-A) or (I-B)), zinc phosphide+((I-A) or (I-B)), zolaprofos+((I-A) or (I-B)), and meperfluthrin+((I-A) or (I-B)), tetramethylfluthrin+((I-A) or (I-B)), bis(tributyltin) oxide+((I-A) or (I-B)), bromoacetamide+((I-A) or (I-B)), ferric phosphate+((I-A) or (I-B)), niclosamide-olamine+((I-A) or (I-B)), tributyltin oxide+((I-A) or (I-B)), pyrimorph+((I-A) or (I-B)), trifenmorph+((I-A) or (I-B)), 1,2-dibromo-3-chloropropane+((I-A) or (I-B)), 1,3-dichloropropene+((I-A) or (I-B)), 3,4-dichlorotetrahydrothiophene 1,1-dioxide+((I-A) or (I-B)), 3-(4-chlorophenyl)-5-methylrhodanine+((I-A) or (I-B)), 5-methyl-6-thioxo-1,3,5-thiadiazinan-3-ylacetic acid+((I-A) or (I-B)), 6-isopentenylaminopurine+((I-A) or (I-B)), anisiflupurin+((I-A) or (I-B)), benclothiaz+((I-A) or (I-B)), cytokinins+((I-A) or (I-B)), DCIP+((I-A) or (I-B)), furfural+((I-A) or (I-B)), isamidofos+((I-A) or (I-B)), kinetin+((I-A) or (I-B)), Myrothecium verrucaria composition+((I-A) or (I-B)), tetrachlorothiophene+((I-A) or (I-B)), xylenols+((I-A) or (I-B)), zeatin+((I-A) or (I-B)), potassium ethylxanthate+((I-A) or (I-B)), acibenzolar+((I-A) or (I-B)), acibenzolar-S-methyl+((I-A) or (I-B)), Reynoutria sachalinensis extract+((I-A) or (I-B)), alpha-chlorohydrin+((I-A) or (I-B)), antu+((I-A) or (I-B)), barium carbonate+((I-A) or (I-B)), bisthiosemi+((I-A) or (I-B)), brodifacoum+((I-A) or (I-B)), bromadiolone+((I-A) or (I-B)), bromethalin+((I-A) or (I-B)), chlorophacinone+((I-A) or (I-B)), cholecalciferol+((I-A) or (I-B)), coumachlor+((I-A) or (I-B)), coumafuryl+((I-A) or (I-B)), coumatetralyl+((I-A) or (I-B)), crimidine+((I-A) or (I-B)), difenacoum+((I-A) or (I-B)), difethialone+((I-A) or (I-B)), diphacinone+((I-A) or (I-B)), ergocalciferol+((I-A) or (I-B)), flocoumafen+((I-A) or (I-B)), fluoroacetamide+((I-A) or (I-B)), flupropadine+((I-A) or (I-B)), flupropadine hydrochloride+((I-A) or (I-B)), norbormide+((I-A) or (I-B)), phosacetim+((I-A) or (I-B)), phosphorus+((I-A) or (I-B)), pindone+((I-A) or (I-B)), pyrinuron+((I-A) or (I-B)), scilliroside+((I-A) or (I-B)), sodium fluoroacetate+((I-A) or (I-B)), thallium sulfate+((I-A) or (I-B)), warfarin+((I-A) or (I-B)), 2-(2-butoxyethoxy)ethyl piperonylate+((I-A) or (I-B)), 5-(1,3-benzodioxol-5-yl)-3-hexylcyclohex-2-enone+((I-A) or (I-B)), farnesol with nerolidol+((I-A) or (I-B)), verbutin+((I-A) or (I-B)), MGK 264+((I-A) or (I-B)), piperonyl butoxide+((I-A) or (I-B)), piprotal+((I-A) or (I-B)), propyl isomer+((I-A) or (I-B)), S421+((I-A) or (I-B)), sesamex+((I-A) or (I-B)), sesasmolin+((I-A) or (I-B)), sulfoxide+((I-A) or (I-B)), anthraquinone+((I-A) or (I-B)), copper naphthenate+((I-A) or (I-B)), copper oxychloride+((I-A) or (I-B)), dicyclopentadiene+((I-A) or (I-B)), thiram+((I-A) or (I-B)), zinc naphthenate+((I-A) or (I-B)), ziram+((I-A) or (I-B)), imanin+((I-A) or (I-B)), ribavirin+((I-A) or (I-B)), mercuric oxide+((I-A) or (I-B)), thiophanate-methyl+((I-A) or (I-B)), azaconazole+((I-A) or (I-B)), bitertanol+((I-A) or (I-B)), bromuconazole+((I-A) or (I-B)), cyproconazole+((I-A) or (I-B)), difenoconazole+((I-A) or (I-B)), diniconazole+((I-A) or (I-B)), epoxiconazole+((I-A) or (I-B)), fenbuconazole+((I-A) or (I-B)), fluquinconazole+((I-A) or (I-B)), flusilazole+((I-A) or (I-B)), flutriafol+((I-A) or (I-B)), furametpyr+((I-A) or (I-B)), hexaconazole+((I-A) or (I-B)), imazalil+((I-A) or (I-B)), imibenconazole+((I-A) or (I-B)), ipconazole+((I-A) or (I-B)), metconazole+((I-A) or (I-B)), myclobutanil+((I-A) or (I-B)), paclobutrazole+((I-A) or (I-B)), pefurazoate+((I-A) or (I-B)), penconazole+((I-A) or (I-B)), prothioconazole+((I-A) or (I-B)), pyrifenox+((I-A) or (I-B)), prochloraz+((I-A) or (I-B)), propiconazole+((I-A) or (I-B)), pyrisoxazole+((I-A) or (I-B)), simeconazole+((I-A) or (I-B)), tebuconazole+((I-A) or (I-B)), tetraconazole+((I-A) or (I-B)), triadimefon+((I-A) or (I-B)), triadimenol+((I-A) or (I-B)), triflumizole+((I-A) or (I-B)), triticonazole+((I-A) or (I-B)), ancymidol+((I-A) or (I-B)), fenarimol+((I-A) or (I-B)), nuarimol+((I-A) or (I-B)), bupirimate+((I-A) or (I-B)), dimethirimol+((I-A) or (I-B)), ethirimol+((I-A) or (I-B)), dodemorph+((I-A) or (I-B)), fenpropidin+((I-A) or (I-B)), fenpropimorph+((I-A) or (I-B)), spiroxamine+((I-A) or (I-B)), tridemorph+((I-A) or (I-B)), cyprodinil+((I-A) or (I-B)), mepanipyrim+((I-A) or (I-B)), pyrimethanil+((I-A) or (I-B)), fenpiclonil+((I-A) or (I-B)), fludioxonil+((I-A) or (I-B)), benalaxyl+((I-A) or (I-B)), furalaxyl+((I-A) or (I-B)), metalaxyl−+((I-A) or (I-B)), Rmetalaxyl+((I-A) or (I-B)), ofurace+((I-A) or (I-B)), oxadixyl+((I-A) or (I-B)), carbendazim+((I-A) or (I-B)), debacarb+((I-A) or (I-B)), fuberidazole+((I-A) or (I-B)), thiabendazole+((I-A) or (I-B)), chlozolinate+((I-A) or (I-B)), dichlozoline+((I-A) or (I-B)), myclozoline+((I-A) or (I-B)), procymidone+((I-A) or (I-B)), vinclozoline+((I-A) or (I-B)), boscalid+((I-A) or (I-B)), carboxin+((I-A) or (I-B)), fenfuram+((I-A) or (I-B)), flutolanil+((I-A) or (I-B)), mepronil+((I-A) or (I-B)), oxycarboxin+((I-A) or (I-B)), penthiopyrad+((I-A) or (I-B)), thifluzamide+((I-A) or (I-B)), dodine+((I-A) or (I-B)), iminoctadine+((I-A) or (I-B)), azoxystrobin+((I-A) or (I-B)), dimoxystrobin+((I-A) or (I-B)), enestroburin+((I-A) or (I-B)), fenaminstrobin+((I-A) or (I-B)), flufenoxystrobin+((I-A) or (I-B)), fluoxastrobin+((I-A) or (I-B)), kresoxim-methyl+((I-A) or (I-B)), metominostrobin+((I-A) or (I-B)), trifloxystrobin+((I-A) or (I-B)), orysastrobin+((I-A) or (I-B)), picoxystrobin+((I-A) or (I-B)), pyraclostrobin+((I-A) or (I-B)), pyrametostrobin+((I-A) or (I-B)), pyraoxystrobin+((I-A) or (I-B)), ferbam+((I-A) or (I-B)), mancozeb+((I-A) or (I-B)), maneb+((I-A) or (I-B)), metiram+((I-A) or (I-B)), propineb+((I-A) or (I-B)), zineb+((I-A) or (I-B)), captafol+((I-A) or (I-B)), captan+((I-A) or (I-B)), fluoroimide+((I-A) or (I-B)), folpet+((I-A) or (I-B)), tolylfluanid+((I-A) or (I-B)), bordeaux mixture+((I-A) or (I-B)), copper oxide+((I-A) or (I-B)), mancopper+((I-A) or (I-B)), oxine-copper+((I-A) or (I-B)), nitrothal-isopropyl+((I-A) or (I-B)), edifenphos+((I-A) or (I-B)), iprobenphos+((I-A) or (I-B)), phosdiphen+((I-A) or (I-B)), tolclofos-methyl+((I-A) or (I-B)), anilazine+((I-A) or (I-B)), benthiavalicarb+((I-A) or (I-B)), blasticidin-S+((I-A) or (I-B)), chloroneb+((I-A) or (I-B)), chlorothalonil+((I-A) or (I-B)), cyflufenamid+((I-A) or (I-B)), cymoxanil+((I-A) or (I-B)), cyclobutrifluram+((I-A) or (I-B)), diclocymet+((I-A) or (I-B)), diclomezine+((I-A) or (I-B)), dicloran+((I-A) or (I-B)), diethofencarb+((I-A) or (I-B)), dimethomorph+((I-A) or (I-B)), flumorph+((I-A) or (I-B)), dithianon+((I-A) or (I-B)), ethaboxam+((I-A) or (I-B)), etridiazole+((I-A) or (I-B)), famoxadone+((I-A) or (I-B)), fenamidone+((I-A) or (I-B)), fenoxanil+((I-A) or (I-B)), ferimzone+((I-A) or (I-B)), fluazinam+((I-A) or (I-B)), fluopicolide+((I-A) or (I-B)), flusulfamide+((I-A) or (I-B)), fluxapyroxad+((I-A) or (I-B)), fenhexamid+((I-A) or (I-B)), fosetyl-aluminium+((I-A) or (I-B)), hymexazol+((I-A) or (I-B)), iprovalicarb+((I-A) or (I-B)), cyazofamid+((I-A) or (I-B)), methasulfocarb+((I-A) or (I-B)), metrafenone+((I-A) or (I-B)), pencycuron+((I-A) or (I-B)), phthalide+((I-A) or (I-B)), polyoxins+((I-A) or (I-B)), propamocarb+((I-A) or (I-B)), pyribencarb+((I-A) or (I-B)), proquinazid+((I-A) or (I-B)), pyroquilon+((I-A) or (I-B)), pyriofenone+((I-A) or (I-B)), quinoxyfen+((I-A) or (I-B)), quintozene+((I-A) or (I-B)), tiadinil+((I-A) or (I-B)), triazoxide+((I-A) or (I-B)), tricyclazole+((I-A) or (I-B)), triforine+((I-A) or (I-B)), validamycin+((I-A) or (I-B)), valifenalate+((I-A) or (I-B)), zoxamide+((I-A) or (I-B)), mandipropamid+((I-A) or (I-B)), flubeneteram+((I-A) or (I-B)), isopyrazam+((I-A) or (I-B)), sedaxane+((I-A) or (I-B)), benzovindiflupyr+((I-A) or (I-B)), pydiflumetofen+((I-A) or (I-B)), 2-[2-chloro-4-(4-chlorophenoxy)phenyl]-2-hydroxy-3-(1,2,4-triazol-1-yl)propanoate+((I-A) or (I-B)), methyl 3-[(4-chlorophenyl)methyl]-2-hydroxy-1-methyl-2-(1,2,4-triazol-1-ylmethyl)cyclopentanecarboxylate+((I-A) or (I-B)), 3-difluoromethyl-1-methyl-1H-pyrazole-4-carboxylic acid (3′,4′,5′-trifluoro-biphenyl-2-yl)-amide+((I-A) or (I-B)), isoflucypram+((I-A) or (I-B)), isotianil+((I-A) or (I-B)), dipymetitrone+((I-A) or (I-B)), 6-ethyl-5,7-dioxo-pyrrolo[4,5][1,4]dithiino[1,2-c]isothiazole-3-carbonitrile+((I-A) or (I-B)), 2-(difluoromethyl)-N-[3-ethyl-1,1-dimethyl-indan-4-yl]pyridine-3-carboxamide+((I-A) or (I-B)), 4-(2,6-difluorophenyl)-6-methyl-5-phenyl-pyridazine-3-carbonitrile+((I-A) or (I-B)), (R)-3-(difluoromethyl)-1-methyl-N-[1,1,3-trimethylindan-4-yl]pyrazole-4-carboxamide+((I-A) or (I-B)), 4-(2-bromo-4-fluoro-phenyl)-N-(2-chloro-6-fluoro-phenyl)-2,5-dimethyl-pyrazol-3-amine+((I-A) or (I-B)), 4-(2-bromo-4-fluorophenyl)-N-(2-chloro-6-fluorophenyl)-1,3-dimethyl-1H-pyrazol-5-amine+((I-A) or (I-B)), fluindapyr+((I-A) or (I-B)), coumethoxystrobin (jiaxiangjunzhi)+((I-A) or (I-B)), Ivbenmixianan+((I-A) or (I-B)), dichlobentiazox+((I-A) or (I-B)), mandestrobin+((I-A) or (I-B)), 3-(4,4-difluoro-3,4-dihydro-3,3-dimethylisoquinolin-1-yl)quinolone+((I-A) or (I-B)), 2-[2-fluoro-6-[(8-fluoro-2-methyl-3-quinolyl)oxy]phenyl]propan-2-ol+((I-A) or (I-B)), oxathiapiprolin+((I-A) or (I-B)), tert-butyl N-[6-[[[(1-methyltetrazol-5-yl)-phenyl-methylene]amino]oxymethyl]-2-pyridyl]carbamate+((I-A) or (I-B)), pyraziflumid+((I-A) or (I-B)), inpyrfluxam+((I-A) or (I-B)), trolprocarb+((I-A) or (I-B)), mefentrifluconazole+((I-A) or (I-B)), ipfentrifluconazole+((I-A) or (I-B)), 2-(difluoromethyl)-N-[(3R)-3-ethyl-1,1-dimethyl-indan-4-yl]pyridine-3-carboxamide+((I-A) or (I-B)), N′-(2,5-dimethyl-4-phenoxy-phenyl)-N-ethyl-N-methyl-formamidine+((I-A) or (I-B)), N′-[4-(4,5-dichlorothiazol-2-yl)oxy-2,5-dimethyl-phenyl]-N-ethyl-N-methyl-formamidine+((I-A) or (I-B)), [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+((I-A) or (I-B)), but-3-ynyl N-[6-[[(Z)-[(1-methyltetrazol-5-yl)-phenyl-methylene]amino]oxymethyl]-2-pyridyl]carbamate+((I-A) or (I-B)), methyl N-[[5-[4-(2,4-dimethylphenyl)triazol-2-yl]-2-methyl-phenyl]methyl]carbamate+((I-A) or (I-B)), 3-chloro-6-methyl-5-phenyl-4-(2,4,6-trifluorophenyl)pyridazine+((I-A) or (I-B)), pyridachlometyl+((I-A) or (I-B)), 3-(difluoromethyl)-1-methyl-N-[1,1,3-trimethylindan-4-yl]pyrazole-4-carboxamide+((I-A) or (I-B)), 1-[2-[[1-(4-chlorophenyl)pyrazol-3-yl]oxymethyl]-3-methyl-phenyl]-4-methyl-tetrazol-5-one+((I-A) or (I-B)), 1-methyl-4-[3-methyl-2-[[2-methyl-4-(3,4,5-trimethylpyrazol-1-yl)phenoxy]methyl]phenyl]tetrazol-5-one+((I-A) or (I-B)), aminopyrifen+((I-A) or (I-B)), ametoctradin+((I-A) or (I-B)), amisulbrom+((I-A) or (I-B)), penflufen+((I-A) or (I-B)), (Z,2E)-5-[1-(4-chlorophenyl)pyrazol-3-yl]oxy-2-methoxyimino-N,3-dimethyl-pent-3-enamide+((I-A) or (I-B)), florylpicoxamid+((I-A) or (I-B)), fenpicoxamid+((I-A) or (I-B)), tebufloquin+((I-A) or (I-B)), ipflufenoquin+((I-A) or (I-B)), quinofumelin+((I-A) or (I-B)), isofetamid+((I-A) or (I-B)), N-[2-[2,4-dichloro-phenoxy]phenyl]-3-(difluoromethyl)-1-methyl-pyrazole-4-carboxamide+((I-A) or (I-B)), N-[2-[2-chloro-4-(trifluoromethyl)phenoxy]phenyl]-3-(difluoromethyl)-1-methyl-pyrazole-4-carboxamide+((I-A) or (I-B)), benzothiostrobin+((I-A) or (I-B)), phenamacril+((I-A) or (I-B)), 5-amino-1,3,4-thiadiazole-2-thiol zinc salt (2:1)+((I-A) or (I-B)), fluopyram+((I-A) or (I-B)), flutianil+((I-A) or (I-B)), fluopimomide+((I-A) or (I-B)), pyrapropoyne+((I-A) or (I-B)), picarbutrazox+((I-A) or (I-B)), 2-(difluoromethyl)-N-(3-ethyl-1,1-dimethyl-indan-4-yl)pyridine-3-carboxamide+((I-A) or (I-B)), 2-(difluoromethyl)-N-((3R)-1,1, 3-trimethylindan-4-yl)pyridine-3-carboxamide+((I-A) or (I-B)), 4-[[6-[2-(2,4-difluorophenyl)-1,1-difluoro-2-hydroxy-3-(1,2,4-triazol-1-yl)propyl]-3-pyridyl]oxy]benzonitrile+((I-A) or (I-B)), metyltetraprole+((I-A) or (I-B)), 2-(difluoromethyl)-N-((3R)-1,1, 3-trimethylindan-4-yl)pyridine-3-carboxamide+((I-A) or (I-B)), α-(1, 1-dimethylethyl)-α-[4′-(trifluoromethoxy) [1,1′-biphenyl]-4-yl]-5-pyrimidinemethanol+((I-A) or (I-B)), fluoxapiprolin+((I-A) or (I-B)), enoxastrobin+((I-A) or (I-B)), 4-[[6-[2-(2,4-difluorophenyl)-1,1-difluoro-2-hydroxy-3-(1,2,4-triazol-1-yl)propyl]-3-pyridyl]oxy]benzonitrile+((I-A) or (I-B)), 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+((I-A) or (I-B)), 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+((I-A) or (I-B)), trinexapac+((I-A) or (I-B)), coumoxystrobin+((I-A) or (I-B)), zhongshengmycin+((I-A) or (I-B)), thiodiazole copper+((I-A) or (I-B)), zinc thiazole+((I-A) or (I-B)), amectotractin+((I-A) or (I-B)), iprodione+((I-A) or (I-B)), N-octyl-N′-[2-(octylamino)ethyl]ethane-1,2-diamine+((I-A) or (I-B)); N′-[5-bromo-2-methyl-6-[(1 S)-1-methyl-2-propoxy-ethoxy]-3-pyridyl]-N-ethyl-N-methyl-formamidine+((I-A) or (I-B)), N′-[5-bromo-2-methyl-6-[(1R)-1-methyl-2-propoxy-ethoxy]-3-pyridyl]-N-ethyl-N-methyl-formamidine+((I-A) or (I-B)), N′-[5-bromo-2-methyl-6-(1-methyl-2-propoxy-ethoxy)-3-pyridyl]-N-ethyl-N-methyl-formamidine+((I-A) or (I-B)), N′-[5-chloro-2-methyl-6-(1-methyl-2-propoxy-ethoxy)-3-pyridyl]-N-ethyl-N-methyl-formamidine+((I-A) or (I-B)), N′-[5-bromo-2-methyl-6-(1-methyl-2-propoxy-ethoxy)-3-pyridyl]-N-isopropyl-N-methyl-formamidine+((I-A) or (I-B)) (these compounds may be prepared from the methods described in WO2015/155075); N′-[5-bromo-2-methyl-6-(2-propoxypropoxy)-3-pyridyl]-N-ethyl-N-methyl-formamidine+((I-A) or (I-B)) (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+((I-A) or (I-B)), N′-[4-(1-cyclopropyl-2,2,2-trifluoro-1-hydroxy-ethyl)-5-methoxy-2-methyl-phenyl]-N-isopropyl-N-methyl-formamidine+((I-A) or (I-B)) (these compounds may be prepared from the methods described in WO2018/228896); N-ethyl-N′-[5-methoxy-2-methyl-4-[2-trifluoromethyl)oxetan-2-yl]phenyl]-N-methyl-formamidine+((I-A) or (I-B)), N-ethyl-N′-[5-methoxy-2-methyl-4-[2-trifuoromethyl)tetrahydrofuran-2-yl]phenyl]-N-methyl-formamidine+((I-A) or (I-B)) (these compounds may be prepared from the methods described in WO2019/110427); N-[(1R)-1-benzyl-3-chloro-1-methyl-but-3-enyl]-8-fluoro-quinoline-3-carboxamide+((I-A) or (I-B)), N-[(1S)-1-benzyl-3-chloro-1-methyl-but-3-enyl]-8-fluoro-quinoline-3-carboxamide+((I-A) or (I-B)), N-[(1R)-1-benzyl-3,3,3-trifluoro-1-methyl-propyl]-8-fluoro-quinoline-3-carboxamide+((I-A) or (I-B)), N-[(1S)-1-benzyl-3,3,3-trifluoro-1-methyl-propyl]-8-fluoro-quinoline-3-carboxamide+((I-A) or (I-B)), N-[(1R)-1-benzyl-1,3-dimethyl-butyl]-7,8-difluoro-quinoline-3-carboxamide+((I-A) or (I-B)), N-[(1S)-1-benzyl-1,3-dimethyl-butyl]-7,8-difluoro-quinoline-3-carboxamide+((I-A) or (I-B)), 8-fluoro-N-[(1R)-1-[(3-fluorophenyl)methyl]-1,3-dimethyl-butyl]quinoline-3-carboxamide+((I-A) or (I-B)), 8-fluoro-N-[(1S)-1-[(3-fluorophenyl)methyl]-1,3-dimethyl-butyl]quinoline-3-carboxamide+((I-A) or (I-B)), N-[(1R)-1-benzyl-1,3-dimethyl-butyl]-8-fluoro-quinoline-3-carboxamide+((I-A) or (I-B)), N-[(1S)-1-benzyl-1,3-dimethyl-butyl]-8-fluoro-quinoline-3-carboxamide+((I-A) or (I-B)), N-((1R)-1-benzyl-3-chloro-1-methyl-but-3-enyl)-8-fluoro-quinoline-3-carboxamide+((I-A) or (I-B)), N-((1S)-1-benzyl-3-chloro-1-methyl-but-3-enyl)-8-fluoro-quinoline-3-carboxamide+((I-A) or (I-B)) (these compounds may be prepared from the methods described in WO2017/153380); 1-(6,7-dimethylpyrazolo[1,5-a]pyridin-3-yl)-4,4,5-trifluoro-3,3-dimethyl-isoquinoline+((I-A) or (I-B)), 1-(6,7-dimethylpyrazolo[1,5-a]pyridin-3-yl)-4,4,6-trifluoro-3,3-dimethyl-isoquinoline+((I-A) or (I-B)), 4,4-difluoro-3,3-dimethyl-1-(6-methylpyrazolo[1,5-a]pyridin-3-yl)isoquinoline+((I-A) or (I-B)), 4,4-difluoro-3,3-dimethyl-1-(7-methylpyrazolo[1,5-a]pyridin-3-yl)isoquinoline+((I-A) or (I-B)), 1-(6-chloro-7-methyl-pyrazolo[1,5-a]pyridin-3-yl)-4,4-difluoro-3,3-dimethyl-isoquinoline+((I-A) or (I-B)) (these compounds may be prepared from the methods described in WO2017/025510); 1-(4,5-dimethylbenzimidazol-1-yl)-4,4,5-trifluoro-3,3-dimethyl-isoquinoline+((I-A) or (I-B)), 1-(4,5-dimethylbenzimidazol-1-yl)-4,4-difluoro-3,3-dimethyl-isoquinoline+((I-A) or (I-B)), 6-chloro-4,4-difluoro-3,3-dimethyl-1-(4-methylbenzimidazol-1-yl)isoquinoline+((I-A) or (I-B)), 4,4-difluoro-1-(5-fluoro-4-methyl-benzimidazol-1-yl)-3,3-dimethyl-isoquinoline+((I-A) or (I-B)), 3-(4,4-difluoro-3,3-dimethyl-1-isoquinolyl)-7,8-dihydro-6H-cyclopenta[e]benzimidazole+((I-A) or (I-B)) (these compounds may be prepared from the methods described in WO2016/156085); N-ethyl-2-methyl-N-[[4-[5-(trifluoromethyl)-1,2,4-oxadiazol-3-yl]phenyl]methyl]propanamide+((I-A) or (I-B)), 1-methoxy-3-methyl-1-[[4-[5-(trifluoromethyl)-1,2,4-oxadiazol-3-yl]phenyl]methyl]urea+((I-A) or (I-B)), 1,3-dimethoxy-1-[[4-[5-(trifluoromethyl)-1,2,4-oxadiazol-3-yl]phenyl]methyl]urea+((I-A) or (I-B)), 3-ethyl-1-methoxy-1-[[4-[5-(trifluoromethyl)-1,2,4-oxadiazol-3-yl]phenyl]methyl]urea+((I-A) or (I-B)), N-[[4-[5-(trifluoromethyl)-1,2,4-oxadiazol-3-yl]phenyl]methyl]propanamide+((I-A) or (I-B)), 4,4-dimethyl-2-[[4-[5-(trifluoromethyl)-1,2,4-oxadiazol-3-yl]phenyl]methyl]isoxazolidin-3-one+((I-A) or (I-B)), 5,5-dimethyl-2-[[4-[5-(trifluoromethyl)-1,2,4-oxadiazol-3-yl]phenyl]methyl]isoxazolidin-3-one+((I-A) or (I-B)), ethyl 1-[[4-[5-(trifluoromethyl)-1,2,4-oxadiazol-3-yl]phenyl]methyl]pyrazole-4-carboxylate+((I-A) or (I-B)), N,N-dimethyl-1-[[4-[5-(trifluoromethyl)-1,2,4-oxadiazol-3-yl]phenyl]methyl]-1,2,4-triazol-3-amine+((I-A) or (I-B)). The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+((I-A) or (I-B)) (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+((I-A) or (I-B)) (this compound may be prepared from the methods described in WO 2017/029179); 3-[2-(1-chlorocyclopropyl)-3-(2-fluorophenyl)-2-hydroxy-propyl]imidazole-4-carbonitrile+((I-A) or (I-B)) (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+((I-A) or (I-B)) (this compound may be prepared from the methods described in WO 2016/156290); (4-phenoxyphenyl)methyl 2-amino-6-methyl-pyridine-3-carboxylate+((I-A) or (I-B)) (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+((I-A) or (I-B)) (this compound may be prepared from the methods described in WO 2011/138281); N-methyl-4-[5-(trifluoromethyl)-1,2,4-oxadiazol-3-yl]benzenecarbothioamide+((I-A) or (I-B)); N-methyl-4-[5-(trifluoromethyl)-1,2,4-oxadiazol-3-yl]benzamide+((I-A) or (I-B)); (Z,2E)-5-[1-(2,4-dichlorophenyl)pyrazol-3-yl]oxy-2-methoxyimino-N,3-dimethyl-pent-3-enamide+((I-A) or (I-B)) (this compound may be prepared from the methods described in WO 2018/153707); N′-(2-chloro-5-methyl-4-phenoxy-phenyl)-N-ethyl-N-methyl-formamidine+((I-A) or (I-B)); N′-[2-chloro-4-(2-fluorophenoxy)-5-methyl-phenyl]-N-ethyl-N-methyl-formamidine+((I-A) or (I-B)) (this compound may be prepared from the methods described in WO 2016/202742); 2-(difluoromethyl)-N-[(3S)-3-ethyl-1,1-dimethyl-indan-4-yl]pyridine-3-carboxamide+((I-A) or (I-B)) (this compound may be prepared from the methods described in WO 2014/095675); (5-methyl-2-pyridyl)-[4-[5-(trifluoromethyl)-1,2,4-oxadiazol-3-yl]phenyl]methanone+((I-A) or (I-B)), (3-methylisoxazol-5-yl)-[4-[5-(trifluoromethyl)-1,2,4-oxadiazol-3-yl]phenyl]methanone+((I-A) or (I-B)) (these compounds may be prepared from the methods described in WO 2017/220485); 2-oxo-N-propyl-2-[4-[5-(trifluoromethyl)-1,2,4-oxadiazol-3-yl]phenyl]acetamide+((I-A) or (I-B)) (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+((I-A) or (I-B)) (this compound may be prepared from the methods described in WO 2018/158365); 2,2-difluoro-N-methyl-2-[4-[5-(trifluoromethyl)-1,2,4-oxadiazol-3-yl]phenyl]acetamide+((I-A) or (I-B)), N-[(E)-methoxyiminomethyl]-4-[5-(trifluoromethyl)-1,2,4-oxadiazol-3-yl]benzamide+((I-A) or (I-B)), N-[(Z)-methoxyiminomethyl]-4-[5-(trifluoromethyl)-1,2,4-oxadiazol-3-yl]benzamide+((I-A) or (I-B)), N—[N-methoxy-C-methyl-carbonimidoyl]-4-[5-(trifluoromethyl)-1,2,4-oxadiazol-3-yl]benzamide+((I-A) or (I-B)) (these compounds may be prepared from the methods described in WO 2018/202428), chloroinconazide+((I-A) or (I-B)), flumetylsulforim+((I-A) or (I-B)), fluoxytioconazole+((I-A) or (I-B)), flufenoxadiazam+((I-A) or (I-B)), metarylpicoxamid+((I-A) or (I-B)); 1-[1-(4-chloro phenyl)cyclobutyl]ethyl (2S)-2-[(3-hydroxy-4-methoxy-pyridine-2-carbonyl)-amino]propanoate+((I-A) or (I-B)), [2-(4-bromo-7-fluoro-indol-1-yl)-1-methyl-propyl](2S)-2-[(3-hydroxy-4-methoxy-pyridine-2-carbonyl)amino]propanoate+((I-A) or (I-B)), [2-(3,5-dichloro-2-pyridyl)-1-methyl-propyl](2S)-2-[(3-hydroxy-4-methoxy-pyridine-2-carbonyl)amino]propanoate+((I-A) or (I-B)), [(1S)-1-[1-(1-naphthyl)cyclopropyl]ethyl](2S)-2-[[3-(acetoxymethoxy)-4-methoxy-pyridine-2-carbonyl]amino]propanoate+((I-A) or (I-B)), [(1S)-1-[1-(1-naphthyl)cyclopropyl]ethyl](2S)-2-[(3-hydroxy-4-methoxy-pyridine-2-carbonyl)amino]propanoate+((I-A) or (I-B)), [2-[[(1S)-2-[2-(3,5-dichloro-2-pyridyl)-1-methyl-propoxy]-1-methyl-2-oxo-ethyl]carbamoyl]-4-methoxy-3-pyridyl]oxymethyl 2-methylpropanoate+((I-A) or (I-B)), [4-methoxy-2-[[(1S)-1-methyl-2-[(1S)-1-[1-(1-naphthyl)cyclopropyl]ethoxy]-2-oxo-ethyl]carbamoyl]-3-pyridyl]oxymethyl 2-methylpropanoate+((I-A) or (I-B)), [2-(4-bromophenyl)-1,2-dimethyl-propyl](2S)-2-[(3-hydroxy-4-methoxy-pyridine-2-carbonyl)amino]propanoate+((I-A) or (I-B)), 1-(1-phenyl cyclohexyl)ethyl (2S)-2-[(3-hydroxy-4-methoxy-pyridine-2-carbonyl)amino]propanoate+((I-A) or (I-B)), [1-methyl-2-(2-quinolyl)propyl](2S)-2-[(3-hydroxy-4-methoxy-pyridine-2-carbonyl)amino]propanoate+((I-A) or (I-B)), [2-(7-bromoindol-1-yl)-1-methyl-propyl](2S)-2-[(3-hydroxy-4-methoxy-pyridine-2-carbonyl)amino]propanoate+((I-A) or (I-B)), [1-methyl-2-[6-(trifluoromethyl)indol-1-yl]propyl](2S)-2-[(3-hydroxy-4-methoxy-pyridine-2-carbonyl)amino]propanoate+((I-A) or (I-B)), (2-indazol-1-yl-1-methyl-propyl) (2S)-2-[(3-hydroxy-4-methoxy-pyridine-2-carbonyl)amino]propanoate+((I-A) or (I-B)), [2-(5-chloro-2-thienyl)-1-methyl-propyl](2S)-2-[(3-hydroxy-4-methoxy-pyridine-2-carbonyl)amino]propanoate+((I-A) or (I-B)), [2-(4,7-dichloroindol-1-yl)-1-methyl-propyl](2S)-2-[(3-acetoxy-4-methoxy-pyridine-2-carbonyl)amino]propanoate+((I-A) or (I-B)), [2-(7-bromo-4-fluoro-indol-1-yl)-1-methyl-propyl](2S)-2-[(3-acetoxy-4-methoxy-pyridine-2-carbonyl)amino]propanoate+((I-A) or (I-B)), [(1S)-1-[1-(1-naphthyl)cyclopropyl]ethyl](2S)-2-[(3-acetoxy-4-methoxy-pyridine-2-carbonyl)amino]propanoate+((I-A) or (I-B)), (these compounds may be prepared from the methods described in WO2020/208096); methyl N-[[5-[1-(2,6-difluoro-4-isopropyl-phenyl)pyrazol-3-yl]-2-methyl-phenyl]methyl]carbamate+((I-A) or (I-B)), methyl N-[[5-[1-(4-cyclopropyl-2,6-difluoro-phenyl)pyrazol-3-yl]-2-methyl-phenyl]methyl]carbamate+((I-A) or (I-B)), (2E)-2-methoxyimino-N-methyl-2-[3-methyl-2-[[(E)-1-[4-(trifluoromethyl)-2-pyridyl]ethylideneamino]oxymethyl]phenyl]acetamide+((I-A) or (I-B)), 6-chloro-3-(3-cyclopropyl-2-fluoro-phenoxy)-N-[2-(2,4-dimethylphenyl)-2,2-difluoro-ethyl]-5-methyl-pyridazine-4-carboxamide+((I-A) or (I-B)), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide+((I-A) or (I-B)), 6-chloro-3-(3-cyclopropyl-2-fluoro-phenoxy)-N-[2-(3,4-dimethylphenyl)-2,2-difluoro-ethyl]-5-methyl-pyridazine-4-carboxamide+((I-A) or (I-B)), 5-[5-(difluoromethyl)-1,3,4-oxadiazol-2-yl]-N-[1-(2,6-difluorophenyl)ethyl]pyrimidin-2-amine+((I-A) or (I-B)), 2-(difluoromethyl)-5-[2-[1-(2,6-difluorophenyl)cyclopropoxy]pyrimidin-5-yl]-1,3,4-oxadiazole+((I-A) or (I-B)), 5-[5-(difluoromethyl)-1,3,4-oxadiazol-2-yl]-N-[1-(2,6-difluorophenyl)cyclopropyl]pyrimidin-2-amine+((I-A) or (I-B)), 5-[5-(difluoromethyl)-1,3,4-oxadiazol-2-yl]-N-[1-(2-fluorophenyl)cyclopropyl]pyrimidin-2-amine+((I-A) or (I-B)), N-[1-(2-fluorophenyl)cyclopropyl]-5-[5-(trifluoromethyl)-1,3,4-oxadiazol-2-yl]pyrimidin-2-amine+((I-A) or (I-B)), 5-[5-(difluoromethyl)-1,3,4-oxadiazol-2-yl]-N-[1-(2-fluorophenyl)ethyl]pyrimidin-2-amine+((I-A) or (I-B)), 5-[5-(difluoromethyl)-1,3,4-oxadiazol-2-yl]-N-[1-(3,5-difluorophenyl)ethyl]pyrimidin-2-amine+((I-A) or (I-B)), ethyl 1-[[4-[[2-(trifluoromethyl)-1,3-dioxolan-2-yl]methoxy]phenyl]methyl]-1H-pyrazole-4-carboxylate+((I-A) or (I-B)), ethyl 1-[[4-[[(1Z)-2-ethoxy-3,3,3-trifluoro-1-propen-1-yl]oxy]phenyl]methyl]-1H-pyrazole-4-carboxylate+((I-A) or (I-B)), 3-[[3-chloro-2-(2-ethylpyrazol-3-yl)phenyl]methyl]-7,8-difluoro-2-methyl-quinoline+((I-A) or (I-B)), N-[2-(2-ethylpyrazol-3-yl)phenyl]-5,6-difluoro-3-methyl-quinoxalin-2-amine+((I-A) or (I-B)), 5,6-difluoro-N-[3-fluoro-2-(2-propylpyrazol-3-yl)phenyl]-3-methyl-quinoxalin-2-amine+((I-A) or (I-B)), N-[3-chloro-2-(2-propylpyrazol-3-yl)phenyl]-7,8-difluoro-2-methyl-quinolin-3-amine+((I-A) or (I-B)), 1-(5,6-dimethyl-3-pyridyl)-4,4-difluoro-3,3-dimethyl-isoquinoline+((I-A) or (I-B)), 1-[6-(difluoromethyl)-5-methyl-3-pyridyl]-4,4-difluoro-3,3-dimethyl-isoquinoline+((I-A) or (I-B)), methyl 2-[2-chloro-4-(4-chlorophenoxy)phenyl]-2-hydroxy-3-(1,2,4-triazol-1-yl)propanoate+((I-A) or (I-B)) and 2-(1-chlorocyclopropyl)-1-(2-chlorophenyl)-3-(1,2,4-triazol-1-yl)propan-2-ol+((I-A) or (I-B)).

The references in brackets behind the active ingredients, e.g. [3878-19-1] refer to the Chemical Abstracts Registry number. The above-described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the polymorphs of the invention is preferably in a mixing ratio of from 100:1 to 1:100, especially from 50:1 to 1:50, more especially in a ratio of from 20:1 to 1:20, even more especially from 10:1 to 1:10, and still more especially from 5:1 to 1:5 Those mixing ratios are by weight.

In another aspect to the present invention, there is provided a fungicidal composition comprising a mixture of a polymorph (I-A) or (I-B) according to the first or second aspect as component (A) and a component (B) as active ingredients, wherein component (A) is selected from the polymorph of a compound of formula (I-A), or the polymorph of a compound of formula (I-B) and component (B) is a compound selected from the group consisting of benzovindiflupyr, fluxapyroxad, pydiflumetofen, isopyrazam, fluopyram, penthiopyrad, sedaxane, bixafen, difenoconazole, cyproconazole, tebuconazole, hexaconazole, prothioconazole, propiconazole, epoxiconazole, metconazole, tetraconazole, fluoxytioconazole, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentanecarboxylate, flutriafol, mefentrifluconazole, ipconazole, paclobutrazol, azoxystrobin, trifloxystrobin, picoxystrobin, pyraclostrobin, metalaxyl-M, fenpropidin, fenpropimorph, cyprodinil, spiroxamine, mancozeb, chlorothalonil, folpet, copper-oxychloride, copper-hydroxide, sulphur, oxathiapiprolin, ipflufenoquin, quinofumelin, mandipropamid, fluazinam, fludioxinil, fosetyl-aluminium, acibenzolar-Smethyl, enestrobin, inpyrfluxam, fluindapyr, isoflucypram, metyltetraprole, florylpicoxamid, metarylpicoxamide, flefunoxadiazam isofetamid, procymidone, carbendazim, fenhexamid, prochloraz, prohexadione-calcium, melaluca alternifolia oil (an extract of the tea tree plant Melaluca alternifolia (commercially available as Timorex Gold®, which is a broad-spectrum botanical biofungicide)), N′-[5-bromo-2-methyl-6-(1-methyl-2-propoxy-ethoxy)-3-pyridyl]-N-ethyl-N-methylformamidine, cyclobutrifluram calcium phosphonate, cis-jasmone, trinexapac-ethyl, glyphosate, 2,4-D (2,4-dichlorophenoxyacetic acid) or thiamethoxam, or a salt, enantiomer, tautomer or N-oxide thereof.

In general, the weight ratio of component (A) to component (B) may be from 1000:1 to 1:1000, may be from 100:1 to 1:100, preferably from 50:1 to 1:50, more preferably from 20:1 to 1:40, even more preferably from 15:1 to 1:30, still more preferably from 12:1 to 1:25, or from 10:1 to 1:20, or from 10:1 to 1:10, or from 5:1 and 1:15, or from 5:1 to 1:5, or from 4:1 to 1:4, or from 3:1 to 1:10, or from 3:1 to 1:3, or from 2:1 to 1:5, or 1:1.

In a preferred composition according to this aspect of the invention, component (A) is the polymorph of N-methoxy-N-[[4-[5-(trifluoromethyl)-1,2,4-oxadiazol-3-yl]phenyl]methyl]cyclopropanecarboxamide (compound I-A), or a salt, enantiomer, tautomer or N-oxide thereof, and component (B) is selected from the group consisting of benzovindiflupyr, fluxapyroxad, pydiflumetofen, isopyrazam, fluopyram, penthiopyrad, sedaxane, bixafen, difenoconazole, cyproconazole, tebuconazole, hexaconazole, prothioconazole, propiconazole, epoxiconazole, metconazole, tetraconazole, fluoxytioconazole, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentanecarboxylate, flutriafol, mefentrifluconazole, ipconazole, paclobutrazol, azoxystrobin, trifloxystrobin, picoxystrobin, pyraclostrobin, metalaxyl-M, fenpropidin, fenpropimorph, cyprodinil, spiroxamine, mancozeb, chlorothalonil, folpet, copper-oxychloride, copper-hydroxide, sulphur, oxathiapiprolin, ipflufenoquin, quinofumelin, mandipropamid, fluazinam, fludioxinil, fosetyl-aluminium, acibenzolar-Smethyl, enestrobin, inpyrfluxam, fluindapyr, isoflucypram, metyltetraprole, florylpicoxamid, metarylpicoxamide, flefunoxadiazam isofetamid, procymidone, carbendazim, fenhexamid, prochloraz, prohexadione-calcium, melaluca alternifolia oil (an extract of the tea tree plant Melaluca alternifolia (commercially available as Timorex Gold®, which is a broad-spectrum botanical biofungicide)),N′-[5-bromo-2-methyl-6-(1-methyl-2-propoxy-ethoxy)-3-pyridyl]-N-ethyl-N-methylformamidine, cyclobutrifluram calcium phosphonate, cis-jasmone, trinexapac-ethyl, glyphosate, 2,4-D (2,4-dichlorophenoxyacetic acid) or thiamethoxam, or a salt, enantiomer, tautomer or N-oxide thereof.

In another preferred composition according to the invention, component (A) is the polymorph of N,2-dimethoxy-N-[[4-[5-(trifluoromethyl)-1,2,4-oxadiazol-3-yl]phenyl]methyl]propanamide (compound I-B), or a salt, enantiomer, tautomer or N-oxide thereof, and component (B) is selected from the group consisting of benzovindiflupyr, fluxapyroxad, pydiflumetofen, isopyrazam, fluopyram, penthiopyrad, sedaxane, bixafen, difenoconazole, cyproconazole, tebuconazole, hexaconazole, prothioconazole, propiconazole, epoxiconazole, metconazole, tetraconazole, fluoxytioconazole, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentanecarboxylate, flutriafol, mefentrifluconazole, ipconazole, paclobutrazol, azoxystrobin, trifloxystrobin, picoxystrobin, pyraclostrobin, metalaxyl-M, fenpropidin, fenpropimorph, cyprodinil, spiroxamine, mancozeb, chlorothalonil, folpet, copper-oxychloride, copper-hydroxide, sulphur, oxathiapiprolin, ipflufenoquin, quinofumelin, mandipropamid, fluazinam, fludioxinil, fosetyl-aluminium, acibenzolar-Smethyl, enestrobin, inpyrfluxam, fluindapyr, isoflucypram, metyltetraprole, florylpicoxamid, metarylpicoxamide, flefunoxadiazam isofetamid, procymidone, carbendazim, fenhexamid, prochloraz, prohexadione-calcium, melaluca alternifolia oil (an extract of the tea tree plant Melaluca alternifolia (commercially available as Timorex Gold®, which is a broad-spectrum botanical biofungicide)),N′-[5-bromo-2-methyl-6-(1-methyl-2-propoxy-ethoxy)-3-pyridyl]-N-ethyl-N-methylformamidine, cyclobutrifluram calcium phosphonate, cis-jasmone, trinexapac-ethyl, glyphosate, 2,4-D (2,4-dichlorophenoxyacetic acid) or thiamethoxam, or a salt, enantiomer, tautomer or N-oxide thereof.

In any of the compositions according to the invention, the composition may comprise an additional active ingredient component (C), which is different to component (B), and is selected from the group consisting of metyltetrapole, azoxystrobin, trifloxystrobin, picoxystrobin, pyraclostrobin, metominostrobin, prothioconazole, cyproconazole, difenoconazole, tebuconazole, propiconazole, epoxiconazole, hexaconazole, mefentrifluconazole, metconazole, benzovindiflupyr, pydiflumetofen, fluxapyroxad, sedaxane, bixafen, isopyrazam, fluopyram, fluindapyr, isoflucypram, inpyrfluxam, quinofumelin, ipflufenoquin, aminopyrifen, fluazinam, fludioxonil, fenpicoxamid, florylpicoxamid, fenpropidin, fenpropimorph, mancozeb, chlorothalonil, cyclobutrifluram, copper-oxychloride, copper-hydroxide, N′-[5-bromo-2-methyl-6-(1-methyl-2-propoxy-ethoxy)-3-pyridyl]-N-ethyl-N-methyl-formamidine), N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine, N′-[5-chloro-2-methyl-6-(1-methyl-2-propoxy-ethoxy)-3-pyridyl]-N-ethyl-N-methyl-formamidine, N′-(2-chloro-5-methyl-4-phenoxyphenyl)-N-ethyl-N-methyl-formamidine, N′-[2-chloro-4-(2-fluorophenoxy)-5-methylphenyl]-N-ethyl-N-methyl-formamidine, N′-[2-chloro-4-(3-fluorophenoxy)-5-methylphenyl]-N-ethyl-N-methyl-formamidine, N′-(2-bromo-5-methyl-4-phenoxyphenyl)-N-ethyl-N-methyl-formamidine, N′-[2-bromo-4-(2-fluorophenoxy)-5-methylphenyl]-N-ethyl-N-methyl-formamidine, N′-[2-chloro-4-(2-fluorophenoxy)-5-methylphenyl]-N-isopropyl-N-methyl-formamidine, N′-[4-(2-cyanophenoxy)-2-methyl-5-(trifluoromethyl)phenyl]-N-ethyl-N-methyl-formamidine, N′-[5-bromo-2-methyl-4-(2-methylphenoxy)phenyl]-N-ethyl-N-methyl-formamidine, N-ethyl-N′-[4-(2-fluorophenoxy)-2-methyl-5-(trifluoromethyl)phenyl]-N-methyl-formamidine, (Z,2E)-5-[1-(2,4-dichlorophenyl)pyrazol-3-yl]oxy-2-methoxyimino-N,3-dimethyl-pent-3-enamide, (Z,2E)-5-[1-(4-chlorophenyl)pyrazol-3-yl]oxy-2-methoxyimino-N,3-dimethyl-pent-3-enamide, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, methyl 3-[(4-chlorophenyl)methyl]-2-hydroxy-1-methyl-2-(1,2,4-triazol-1-ylmethyl)cyclopentanecarboxylate, 2-[6-(4-bromophenoxy)-2-(trifluoromethyl)-3-pyridyl]-1-(1,2,4-triazol-1-yl)propan-2-ol, 2-[6-(4-chlorophenoxy)-2-(trifluoromethyl)-3-pyridyl]-1-(1,2,4-triazol-1-yl)propan-2-ol, N-methyl-4-[5-(trifluoromethyl)-1,2,4-oxadiazol-3-yl]benzenecarbothioamide, N-methyl-4-[5-(trifluoromethyl)-1,2,4-oxadiazol-3-yl]benzamide, 2,2-difluoro-N-methyl-2-[4-[5-(trifluoromethyl)-1,2,4-oxadiazol-3-yl]phenyl]acetamide, N-[N-methoxy-C-methyl-carbonimidoyl]-4-[5-(trifluoromethyl)-1,2,4-oxadiazol-3-yl]benzamide, N-[(E)-methoxyiminomethyl]-4-[5-(trifluoromethyl)-1,2,4-oxadiazol-3-yl]benzamide, N-[4-[5-(trifluoromethyl)-1,2,4-oxadiazol-3-yl]phenyl]cyclopropanecarboxamide, N-(2-fluorophenyl)-4-[5-(trifluoromethyl)-1,2,4-oxadiazol-3-yl]benzamide, 1-(5,6-dimethyl-3-pyridyl)-4,4-difluoro-3,3-dimethyl-isoquinoline and 1-[6-(difluoromethyl)-5-methyl-3-pyridyl]-4,4-difluoro-3,3-dimethyl-isoquinoline.

The component (C) compounds are referred to herein and above by a so-called “ISO common name” or another “common name” being used in individual cases or a trademark name. The component (C) compounds are known and are commercially available and/or can be prepared using procedures known in the art

In another aspect to the present invention, there is provided a fungicidal composition comprising a mixture of a polymorph according to the first or second aspect (A) and a component (B) and a component (C) as active ingredients, wherein component (A) is selected from the polymorph of a compound of formula (I-A), or the polymorph of a compound of formula (I-B) and component (B) and (C) are a compound selected from the group consisting of benzovindiflupyr, fluxapyroxad, pydiflumetofen, isopyrazam, fluopyram, penthiopyrad, sedaxane, bixafen, difenoconazole, cyproconazole, tebuconazole, hexaconazole, prothioconazole, propiconazole, epoxiconazole, metconazole, tetraconazole, fluoxytioconazole, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentanecarboxylate, flutriafol, mefentrifluconazole, ipconazole, paclobutrazol, azoxystrobin, trifloxystrobin, picoxystrobin, pyraclostrobin, metalaxyl-M, fenpropidin, fenpropimorph, cyprodinil, spiroxamine, mancozeb, chlorothalonil, folpet, copper-oxychloride, copper-hydroxide, sulphur, oxathiapiprolin, ipflufenoquin, quinofumelin, mandipropamid, fluazinam, fludioxinil, fosetyl-aluminium, acibenzolar-Smethyl, enestrobin, inpyrfluxam, fluindapyr, isoflucypram, metyltetraprole, florylpicoxamid, metarylpicoxamide, flefunoxadiazam isofetamid, procymidone, carbendazim, fenhexamid, prochloraz, prohexadione-calcium, melaluca alternifolia oil (an extract of the tea tree plant Melaluca alternifolia (commercially available as Timorex Gold®, which is a broad-spectrum botanical biofungicide)),N′-[5-bromo-2-methyl-6-(1-methyl-2-propoxy-ethoxy)-3-pyridyl]-N-ethyl-N-methylformamidine, cyclobutrifluram calcium phosphonate, cis-jasmone, trinexapac-ethyl, glyphosate, 2,4-D (2,4-dichlorophenoxyacetic acid) or thiamethoxam, or a salt, enantiomer, tautomer or N-oxide thereof, wherein component (B) and (C) are not the same compound.

In a preferred composition according to this aspect of the invention, component (A) is the polymorph of N-methoxy-N-[[4-[5-(trifluoromethyl)-1,2,4-oxadiazol-3-yl]phenyl]methyl]cyclopropanecarboxamide (compound I-A), or a salt, enantiomer, tautomer or N-oxide thereof, and component (B) and (C) are a compound selected from the group consisting of benzovindiflupyr, fluxapyroxad, pydiflumetofen, isopyrazam, fluopyram, penthiopyrad, sedaxane, bixafen, difenoconazole, cyproconazole, tebuconazole, hexaconazole, prothioconazole, propiconazole, epoxiconazole, metconazole, tetraconazole, fluoxytioconazole, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentane carboxylate, flutriafol, mefentrifluconazole, ipconazole, paclobutrazol, azoxystrobin, trifloxystrobin, picoxystrobin, pyraclostrobin, metalaxyl-M, fenpropidin, fenpropimorph, cyprodinil, spiroxamine, mancozeb, chlorothalonil, folpet, copper-oxychloride, copper-hydroxide, sulphur, oxathiapiprolin, ipflufenoquin, quinofumelin, mandipropamid, fluazinam, fludioxinil, fosetyl-aluminium, acibenzolar-Smethyl, enestrobin, inpyrfluxam, fluindapyr, isoflucypram, metyltetraprole, florylpicoxamid, metarylpicoxamide, flefunoxadiazam isofetamid, procymidone, carbendazim, fenhexamid, prochloraz, prohexadione-calcium, melaluca alternifolia oil (an extract of the tea tree plant Melaluca alternifolia (commercially available as Timorex Gold®, which is a broad-spectrum botanical biofungicide)),N′-[5-bromo-2-methyl-6-(1-methyl-2-propoxy-ethoxy)-3-pyridyl]-N-ethyl-N-methylformamidine, cyclobutrifluram calcium phosphonate, cis-jasmone, trinexapac-ethyl, glyphosate, 2,4-D (2,4-dichlorophenoxyacetic acid) or thiamethoxam, or a salt, enantiomer, tautomer or N-oxide thereof, wherein component (B) and (C) are not the same compound.

In another preferred composition according to this aspect of the invention, component (A) is the polymorph of N,2-dimethoxy-N-[[4-[5-(trifluoromethyl)-1,2,4-oxadiazol-3-yl]phenyl]methyl]propanamide (compound I-B), or a salt, enantiomer, tautomer or N-oxide thereof, and component (B) and (C) are a compound selected from the group consisting of benzovindiflupyr, fluxapyroxad, pydiflumetofen, isopyrazam, fluopyram, penthiopyrad, sedaxane, bixafen, difenoconazole, cyproconazole, tebuconazole, hexaconazole, prothioconazole, propiconazole, epoxiconazole, metconazole, tetraconazole, fluoxytioconazole, 2-[2-chloro-4-(4-chlorophenoxy)phenyl]-2-hydroxy-3-(1,2,4-triazol-1-yl)propanoate, methyl 3-[(4-chlorophenyl)methyl]-2-hydroxy-1-methyl-2-(1,2,4-triazol-1-ylmethyl)cyclopentanecarboxylate, flutriafol, mefentrifluconazole, ipconazole, paclobutrazol, azoxystrobin, trifloxystrobin, picoxystrobin, pyraclostrobin, metalaxyl-M, fenpropidin, fenpropimorph, cyprodinil, spiroxamine, mancozeb, chlorothalonil, folpet, copper-oxychloride, copper-hydroxide, sulphur, oxathiapiprolin, ipflufenoquin, quinofumelin, mandipropamid, fluazinam, fludioxinil, fosetyl-aluminium, acibenzolar-Smethyl, enestrobin, inpyrfluxam, fluindapyr, isoflucypram, metyltetraprole, florylpicoxamid, metarylpicoxamide, flefunoxadiazam isofetamid, procymidone, carbendazim, fenhexamid, prochloraz, prohexadione-calcium, melaluca alternifolia oil (an extract of the tea tree plant Melaluca alternifolia (commercially available as Timorex Gold®, which is a broad-spectrum botanical biofungicide)),N′-[5-bromo-2-methyl-6-(1-methyl-2-propoxy-ethoxy)-3-pyridyl]-N-ethyl-N-methylformamidine, cyclobutrifluram calcium phosphonate, cis-jasmone, trinexapac-ethyl, glyphosate, 2,4-D (2,4-dichlorophenoxyacetic acid) or thiamethoxam, or a salt, enantiomer, tautomer or N-oxide thereof, wherein component (B) and (C) are not the same compound.

Components (B) and (C) in combination with component (A) may enhance the effectiveness of the latter against fungi, and vice versa. Additionally, the fungicidal compositions may be effective against a wider spectrum of fungal pathogens that can be combated with the individual active ingredients when used solely. Generally, the weight ratio of component (A) to the mixture of components (B) and (C) may be from 100:1 to 1:100, or 50:1 to 1:50, or 20:1 to 1:20, or 10:1 to 1:10, or 5:1 and 1:5. Otherwise, the weight ratio of component (A) to the mixture of components (B) and (C) may be from 2:1 to 1:2, or 4:1 to 2:1, or 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are understood to include, on the one hand, ratios by weight and also, on other hand, molar ratios.

In embodiments of the invention where the composition comprise a component (A), a component (B) and a component (C), the weight ratio of component (A) to the sum of component (B) and component (C) may be from 100:1 to 1:100, preferably from 50:1 to 1:50, more preferably from 20:1 to 1:40, even more preferably from 15:1 to 1:30, still more preferably from 12:1 to 1:25, or from 10:1 to 1:20, or from 10:1 to 1:10, or from 5:1 and 1:15, or from 5:1 to 1:5, or from 4:1 to 1:4, or from 3:1 to 1:10, or from 3:1 to 1:3, or from 2:1 to 1:5, or 1:1.

In a preferred embodiment of the invention, the polymorph of compound of formula (I-A) or (I-B) according to the invention may be admixed with one or more additional active ingredients selected from component (B), wherein said component (B) is selected from the group consisting of metyltetrapole, azoxystrobin, trifloxystrobin, picoxystrobin, pyraclostrobin, metominostrobin, prothioconazole, cyproconazole, difenoconazole, tebuconazole, propiconazole, epoxiconazole, hexaconazole, mefentrifluconazole, metconazole, benzovindiflupyr, pydiflumetofen, fluxapyroxad, sedaxane, bixafen, isopyrazam, fluopyram, fluindapyr, isoflucypram, inpyrfluxam, quinofumelin, ipflufenoquin, aminopyrifen, fluazinam, fludioxonil, fenpicoxamid, florylpicoxamid, fenpropidin, fenpropimorph, mancozeb, chlorothalonil, cyclobutrifluram, Cu-oxychloride, Cu-hydroxide, N′-[5-bromo-2-methyl-6-(1-methyl-2-propoxy-ethoxy)-3-pyridyl]-N-ethyl-N-methyl-formamidine), N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine, N′-[5-chloro-2-methyl-6-(1-methyl-2-propoxy-ethoxy)-3-pyridyl]-N-ethyl-N-methyl-formamidine, N′-(2-chloro-5-methyl-4-phenoxyphenyl)-N-ethyl-N-methyl-formamidine, N′-[2-chloro-4-(2-fluorophenoxy)-5-methylphenyl]-N-ethyl-N-methyl-formamidine, N′-[2-chloro-4-(3-fluorophenoxy)-5-methylphenyl]-N-ethyl-N-methyl-formamidine, N′-(2-bromo-5-methyl-4-phenoxyphenyl)-N-ethyl-N-methyl-formamidine, N′-[2-bromo-4-(2-fluorophenoxy)-5-methylphenyl]-N-ethyl-N-methyl-formamidine, N′-[2-chloro-4-(2-fluorophenoxy)-5-methylphenyl]-N-isopropyl-N-methyl-formamidine, N′-[4-(2-cyanophenoxy)-2-methyl-5-(trifluoromethyl)phenyl]-N-ethyl-N-methyl-formamidine, N′-[5-bromo-2-methyl-4-(2-methylphenoxy)phenyl]-N-ethyl-N-methyl-formamidine, N-ethyl-N′-[4-(2-fluorophenoxy)-2-methyl-5-(trifluoromethyl)phenyl]-N-methyl-formamidine, (Z,2E)-5-[1-(2,4-dichlorophenyl)pyrazol-3-yl]oxy-2-methoxyimino-N,3-dimethyl-pent-3-enamide, (Z,2E)-5-[1-(4-chlorophenyl)pyrazol-3-yl]oxy-2-methoxyimino-N,3-dimethyl-pent-3-enamide, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, methyl 3-[(4-chlorophenyl)methyl]-2-hydroxy-1-methyl-2-(1,2,4-triazol-1-ylmethyl)cyclopentanecarboxylate, 2-[6-(4-bromophenoxy)-2-(trifluoromethyl)-3-pyridyl]-1-(1,2,4-triazol-1-yl)propan-2-ol, 2-[6-(4-chlorophenoxy)-2-(trifluoromethyl)-3-pyridyl]-1-(1,2,4-triazol-1-yl)propan-2-ol, N-methyl-4-[5-(trifluoromethyl)-1,2,4-oxadiazol-3-yl]benzenecarbothioamide, N-methyl-4-[5-(trifluoromethyl)-1,2,4-oxadiazol-3-yl]benzamide, 2,2-difluoro-N-methyl-2-[4-[5-(trifluoromethyl)-1,2,4-oxadiazol-3-yl]phenyl]acetamide, N-[N-methoxy-C-methyl-carbonimidoyl]-4-[5-(trifluoromethyl)-1,2,4-oxadiazol-3-yl]benzamide, N-[(E)-methoxyiminomethyl]-4-[5-(trifluoromethyl)-1,2,4-oxadiazol-3-yl]benzamide, N-[4-[5-(trifluoromethyl)-1,2,4-oxadiazol-3-yl]phenyl]cyclopropanecarboxamide, N-(2-fluorophenyl)-4-[5-(trifluoromethyl)-1,2,4-oxadiazol-3-yl]benzamide, 1-(5,6-dimethyl-3-pyridyl)-4,4-difluoro-3,3-dimethyl-isoquinoline, 1-[6-(difluoromethyl)-5-methyl-3-pyridyl]-4,4-difluoro-3,3-dimethyl-isoquinoline, methyl N-[[5-[1-(4-cyclopropyl-2,6-difluoro-phenyl)pyrazol-3-yl]-2-methyl-phenyl]methyl]carbamate (this compound may be prepared from the methods described in WO 2021/226234), methyl N-[[5-[1-(2,6-difluoro-4-isopropyl-phenyl)pyrazol-3-yl]-2-methyl-phenyl]methyl]carbamate (this compound may be prepared from the methods described in WO 2021/226234), or [(2S,3S)-3-(2-methylphenyl)butan-2-yl](2S)-2-[(4-methoxy-3-propanoyloxypyridine-2-carbonyl)amino]propanoate (metarylpicoxamide, this compound may be prepared from the methods described in WO2019/173665).

Preferably, component (B), is a compound selected from the group consisting of metyltetrapole, azoxystrobin, trifloxystrobin, picoxystrobin, metominostrobin, prothioconazole, cyproconazole, difenoconazole, tebuconazole, propiconazole, mefentrifluconazole, metconazole, benzovindiflupyr, pydiflumetofen, sedaxane, isopyrazam, inpyrfluxam, fluazinam, florylpicoxamid, fenpropidin, mancozeb, chlorothalonil, cyclobutrifluram, Cu-oxychloride, Cu-hydroxide, N′-[5-bromo-2-methyl-6-(1-methyl-2-propoxy-ethoxy)-3-pyridyl]-N-ethyl-N-methyl-formamidine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, methyl N-[[5-[1-(2,6-difluoro-4-isopropyl-phenyl)pyrazol-3-yl]-2-methyl-phenyl]methyl]carbamate (this compound may be prepared from the methods described in WO 2021/226234), or [(2S,3S)-3-(2-methylphenyl)butan-2-yl](2S)-2-[(4-methoxy-3-propanoyloxypyridine-2-carbonyl)amino]propanoate (metarylpicoxamide, this compound may be prepared from the methods described in WO2019/173665).

According to another aspect of the present invention, there is provided a fungicidal composition comprising a mixture of components (A) and (B) as active ingredients, wherein component (A) is N-methoxy-N-[[4-[5-(trifluoromethyl)-1,2,4-oxadiazol-3-yl]phenyl]methyl]cyclopropanecarboxamide (compound I-A) and component (B) is a compound selected from the group consisting of metyltetrapole, azoxystrobin, trifloxystrobin, picoxystrobin, metominostrobin, prothioconazole, cyproconazole, difenoconazole, tebuconazole, propiconazole, mefentrifluconazole, metconazole, benzovindiflupyr, pydiflumetofen, sedaxane, isopyrazam, inpyrfluxam, fluazinam, florylpicoxamid, fenpropidin, mancozeb, chlorothalonil, cyclobutrifluram, Cu-oxychloride, Cu-hydroxide, N′-[5-bromo-2-methyl-6-(1-methyl-2-propoxy-ethoxy)-3-pyridyl]-N-ethyl-N-methyl-formamidine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, methyl N-[[5-[1-(2,6-difluoro-4-isopropyl-phenyl)pyrazol-3-yl]-2-methyl-phenyl]methyl]carbamate (this compound may be prepared from the methods described in WO 2021/226234), or [(2S,3S)-3-(2-methylphenyl)butan-2-yl](2S)-2-[(4-methoxy-3-propanoyloxypyridine-2-carbonyl)amino]propanoate (metarylpicoxamide, this compound may be prepared from the methods described in WO2019/173665) for controlling phytopathogenic fungi in genetically modified soybean plants. Preferably for controlling Phakopsora pachyrhizi in genetically modified soybean plants. In one embodiment said genetically modified soybean plants are Bt soybean plants, selected from Intacta RR2 PRO®, or Conkesta Enlist E3®.

According to another aspect of the present invention, there is provided a fungicidal composition comprising a mixture of components (A) and (B) as active ingredients, wherein component (A) is N,2-dimethoxy-N-[[4-[5-(trifluoromethyl)-1,2,4-oxadiazol-3-yl]phenyl]methyl]propanamide (compound I-B) and component (B) is a compound selected from the group consisting of metyltetrapole, azoxystrobin, trifloxystrobin, picoxystrobin, metominostrobin, prothioconazole, cyproconazole, difenoconazole, tebuconazole, propiconazole, mefentrifluconazole, metconazole, benzovindiflupyr, pydiflumetofen, sedaxane, isopyrazam, inpyrfluxam, fluazinam, florylpicoxamid, fenpropidin, mancozeb, chlorothalonil, cyclobutrifluram, Cu-oxychloride, Cu-hydroxide, N′-[5-bromo-2-methyl-6-(1-methyl-2-propoxy-ethoxy)-3-pyridyl]-N-ethyl-N-methyl-formamidine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, methyl N-[[5-[1-(2,6-difluoro-4-isopropyl-phenyl)pyrazol-3-yl]-2-methyl-phenyl]methyl]carbamate (this compound may be prepared from the methods described in WO 2021/226234), or [(2S,3S)-3-(2-methylphenyl)butan-2-yl](2S)-2-[(4-methoxy-3-propanoyloxypyridine-2-carbonyl)amino]propanoate (metarylpicoxamide, this compound may be prepared from the methods described in WO2019/173665) for controlling phytopathogenic fungi in genetically modified soybean plants. Preferably for controlling Phakopsora pachyrhizi in genetically modified soybean plants. In one embodiment said genetically modified soybean plants are Bt soybean plants, selected from Intacta RR2 PRO®, or Conkesta Enlist E3®.

According to another aspect of the present invention, there is provided a fungicidal composition comprising a mixture of components (A) and (B) as active ingredients, wherein component (A) is the polymorph of N-methoxy-N-[[4-[5-(trifluoromethyl)-1,2,4-oxadiazol-3-yl]phenyl]methyl]cyclopropanecarboxamide (compound I-A), or a salt, enantiomer, tautomer or N-oxide thereof, and component (B) is a compound selected from the group consisting of metyltetrapole, azoxystrobin, trifloxystrobin, picoxystrobin, metominostrobin, prothioconazole, cyproconazole, difenoconazole, tebuconazole, propiconazole, mefentrifluconazole, metconazole, benzovindiflupyr, pydiflumetofen, sedaxane, isopyrazam, inpyrfluxam, fluazinam, florylpicoxamid, fenpropidin, mancozeb, chlorothalonil, cyclobutrifluram, Cu-oxychloride, Cu-hydroxide, N′-[5-bromo-2-methyl-6-(1-methyl-2-propoxy-ethoxy)-3-pyridyl]-N-ethyl-N-methyl-formamidine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, methyl N-[[5-[1-(2,6-difluoro-4-isopropyl-phenyl)pyrazol-3-yl]-2-methyl-phenyl]methyl]carbamate (this compound may be prepared from the methods described in WO 2021/226234), or [(2S,3S)-3-(2-methylphenyl)butan-2-yl](2S)-2-[(4-methoxy-3-propanoyloxypyridine-2-carbonyl)amino]propanoate (metarylpicoxamide, this compound may be prepared from the methods described in WO2019/173665) for controlling phytopathogenic fungi in genetically modified soybean plants. Preferably for controlling Phakopsora pachyrhizi in genetically modified soybean plants. In one embodiment said genetically modified soybean plants are Bt soybean plants, selected from Intacta RR2 PRO®, or Conkesta Enlist E3®.

According to another aspect of the present invention, there is provided a fungicidal composition comprising a mixture of components (A) and (B) as active ingredients, wherein component (A) is a polymorph of N,2-dimethoxy-N-[[4-[5-(trifluoromethyl)-1,2,4-oxadiazol-3-yl]phenyl]methyl]propanamide (compound I-B), or a salt, enantiomer, tautomer or N-oxide thereof (I-B) and component (B) is a compound selected from the group consisting of metyltetrapole, azoxystrobin, trifloxystrobin, picoxystrobin, metominostrobin, prothioconazole, cyproconazole, difenoconazole, tebuconazole, propiconazole, mefentrifluconazole, metconazole, benzovindiflupyr, pydiflumetofen, sedaxane, isopyrazam, inpyrfluxam, fluazinam, florylpicoxamid, fenpropidin, mancozeb, chlorothalonil, cyclobutrifluram, Cu-oxychloride, Cu-hydroxide, N′-[5-bromo-2-methyl-6-(1-methyl-2-propoxy-ethoxy)-3-pyridyl]-N-ethyl-N-methyl-formamidine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, methyl N-[[5-[1-(2,6-difluoro-4-isopropyl-phenyl)pyrazol-3-yl]-2-methyl-phenyl]methyl]carbamate (this compound may be prepared from the methods described in WO 2021/226234), or [(2S,3S)-3-(2-methylphenyl)butan-2-yl](2S)-2-[(4-methoxy-3-propanoyloxypyridine-2-carbonyl)amino]propanoate (metarylpicoxamide, this compound may be prepared from the methods described in WO2019/173665) for controlling phytopathogenic fungi in genetically modified soybean plants. Preferably for controlling Phakopsora pachyrhizi in genetically modified soybean plants. In one embodiment said genetically modified soybean plants are Bt soybean plants, selected from Intacta RR2 PRO®, or Conkesta Enlist E3®.

The component (B) compounds are referred to herein and above by a so-called “ISO common name” or another “common name” being used in individual cases or a trademark name. The component (B) compounds are known and are commercially available and/or can be prepared using procedures known in the art and/or procedures reported in the literature such as, for instance, WO 2015/155075 and WO 2016/202742, WO 2017/005710, WO 2018/108977, WO 2018/153707, WO 2018/098216, WO 2019/093522, WO 2018/145921, WO 2015/185485, WO 2017/076742, WO 2017/081311, WO 2017/085100, WO 2017/093019, WO 2017/178245 and WO 2017/211649.

In embodiments of the invention where the composition comprise a polymorph of compound of formula (I-A) or (I-B), a first component (B) and a second component (B), wherein said second component (B) is different than said first component (B), the weight ratio of component (A) to the sum of first component (B) and second component (B) may be from 100:1 to 1:100, preferably from 50:1 to 1:50, more preferably from 20:1 to 1:40, even more preferably from 15:1 to 1:30, still more preferably from 12:1 to 1:25, or from 10:1 to 1:20, or from 10:1 to 1:10, or from 5:1 and 1:15, or from 5:1 to 1:5, or from 4:1 to 1:4, or from 3:1 to 1:10, or from 3:1 to 1:3, or from 2:1 to 1:5, or 1:1.

Throughout this document the expression “composition” stands for the various mixtures or combinations of polymorphs of compound (I-A) or (I-B) and components (B) (including the above-defined embodiments), for example in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the components (I-A) or (I-B) and (B) is not essential for working the present invention.

The mixtures as described above can be used in a method for controlling pests, which comprises applying a composition comprising a mixture as described above to the pests or their environment, with the exception of a method for treatment of the human or animal body by surgery or therapy and diagnostic methods practiced on the human or animal body.

The mixtures comprising a polymorph according to the first or second aspect, and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the polymorph according to the invention and the active ingredient(s) as described above, is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of the invention is related to the use of a polymorphs of formula (I-A) or (I-B) according to the invention, of a composition comprising a polymorph of compound of formula (I-A) or (I-B), or of a fungicidal or insecticidal mixture comprising a polymorph of compound of formula (I-A) or (I-B), in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a polymorph of compound of formula (I-A) or (I-B) according to the invention as defined herein as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a polymorph of compound of formula (I-A) or (I-B) at according to the invention, or an agrochemical composition which contains a polymorph of compound of formula (I-A) or (I-B), is foliar application. The frequency of application and the rate of application will depend on the risk of infestation by the corresponding pathogen or insect. However, the polymorph according to the invention can also penetrate the plant through the roots via the soil (systemic action) by drenching the locus of the plant with a liquid formulation, or by applying the compounds in solid form to the soil, e.g., in granular form (soil application). In crops of water rice such granulates can be applied to the flooded rice field. The polymorph may also be applied to seeds (coating) by impregnating the seeds or tubers either with a liquid formulation of the fungicide or coating them with a solid formulation.

A formulation, e.g., a composition containing the polymorph according to the invention and, if desired, a solid or liquid adjuvant or monomers for encapsulating the polymorph of compound of formula (I-A) or (I-B), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of a polymorph per kg of seed, preferably from 0.01 to 10 g per kg of seed are generally sufficient.

Suitably, a composition comprising a polymorph according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g., by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g., as an aqueous suspension or in a dry powder form having good adherence to the seeds. Such seed dressing formulations are known in the art. Seed dressing formulations may contain the single active ingredients or the combination of active ingredients in encapsulated form, e.g., as slow-release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of a polymorph according to the invention optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline: compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

### DESCRIPTION OF DRAWINGS

FIG. 1 shows the measured powder X-ray diffraction pattern of compound (I-A).

FIG. 2 shows the predicted powder X-ray diffraction pattern of compound (I-A).

FIG. 3 shows a DSC (DIFFERENTIAL SCANNING CALORIMETRY) trace of compound (I-A).

FIG. 4 shows the measured powder X-ray diffraction pattern of compound (I-B).

FIG. 5 shows the predicted powder X-ray diffraction pattern of compound (I-B).

FIG. 6 shows a DSC (DIFFERENTIAL SCANNING CALORIMETRY) trace of compound (I-B).

### ABBREVIATIONS

DSC differential scanning calorimetry
MIBK methyl isobutyl ketone
TFAC trifluoroacetylchloride
TMS tetramethylsilan

### EXAMPLES

Throughout this description, temperatures are given in degrees Celsius (° C.) and “mp.” means melting point. ¹H and ¹⁹F NMR measurements were recorded on a Bruker 400 MHz spectrometer, chemical shifts are given in ppm relevant to a TMS (¹H) and CFCl₃ (¹⁹F) standard.

### Preparation Examples

The Examples which follow serve to illustrate the invention. The compounds of formula (I-A) or (I-B) may be prepared according to the synthetic techniques described above.

The compounds of formula (I-A) and (I-B) were made according to the methods described in WO2017/055473, WO2018/177894 and in WO2020/212513.

### Example 1: Preparation of Polymorph of Compound (I-A)

This example illustrates the preparation of the polymorph of N-methoxy-N-[[4-[5-(trifluoromethyl)-1,2,4-oxadiazol-3-yl]phenyl]methyl]cyclopropanecarboxamide (Compound (I-A)).

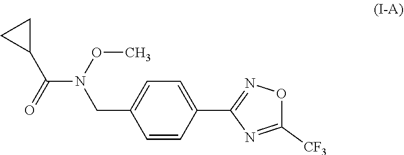 [SMILES: CON(Cc1ccc(-c2noc(C(F)(F)F)n2)cc1)C(=O)C1CC1]

Methods for preparing compound of formula (I-A) has been disclosed in WO2018/177894 (Example 8b) and in WO2020/212513 (Example 1).

### 1a) Preparation of Compound (I-A)

To a 1500 mL flask was added N-[[4-(N-hydroxycarbamimidoyl)phenyl]methyl]-N-methoxycyclopropane carboxamide (93% purity, 112.9 g, 0.4 mol), MIBK (667 g) and 3,5-lutidine (145.0 g, 1.28 mol). To the resultant suspension, TFAC (116.5 g, 0.88 mol) was added over 30 minutes whilst cooling the reaction mixture to 35-40° C. After 1 hour, water (100 mL) was introduced and the contents were stirred for 10 minutes, the layers were separated, and the organic layer was washed with water (200 mL), aq. NaHCO₃ (2×200 g) and water (3×100 g). The organic layer was concentrated under reduced pressure to afford 145 g (99% yield, 93% purity) of compound (I-A) as a yellow oil. Occasionally the oil solidified upon storage, in an uncontrolled manner.

¹H NMR (400 MHz, CDCl₃) δ ppm: 8.09 (d, 2H), 7.53 (d, 2H), 4.87 (s, 2H), 3.73 (s, 3H), 2.19 (m, 1H), 1.05 (m, 2H), 0.86 (m, 2H). ¹⁹F NMR (400 MHz, CDCl₃) δ ppm: −65.33 (s).

### 1b) Preparation of Crystal Seeds of Compound of Formula (I-A)

The oil as obtained under 1a) was dissolved in methylcyclohexane, the resulting mixture heated to 70° C. until complete solution of the oil and the resulting solution was cooled slowly to room temperature and held at this temperature until crystal formation was visible. The solution was further cooled to −5° C. The obtained crystals were filtered off and washed with methylcyclohexane to obtain compound (I-A) as a white crystalline product.

### 1c) Preparation of Polymorph of Compound (I-A)

The oil as obtained under 1a) was dissolved in methylcyclohexane, the resulting mixture heated to 70° C. until complete solution of the oil and the resulting solution was cooled to 10° C. and seed crystals were added. After crystallization, the solution was further cooled to −5° C. and stirred for 30 min. The obtained crystals were filtered and washed with methylcyclohexane to obtain a white crystalline product (98.5% purity). Single-crystal X-ray diffraction analysis confirmed the presence of the polymorph of compound (I-A) of the invention.

### Example 2: Preparation of Polymorph of Compound (I-B)

This example illustrates the preparation of the polymorph of N,2-dimethoxy-N-[[4-[5-(trifluoromethyl)-1,2,4-oxadiazol-3-yl]phenyl]methyl]propanamide (Compound I-B).

Methods for preparing compound of formula (I-B) has been disclosed in WO2018/177894 (Example 9b) and in WO2020/212513. An example for preparing the polymorph of compound of formula (I-B) is as follows:

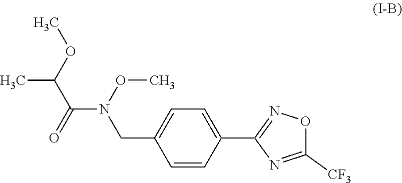 [SMILES: COC(C)C(=O)N(Cc1ccc(-c2noc(C(F)(F)F)n2)cc1)OC]

### 2a) Preparation of Compound (I-B)

To a 250 mL reactor was added N-[[4-(N-hydroxycarbamimidoyl)phenyl]methyl]-N,2-dimethoxy-propanamide (96.7% purity, 50.0 g, 0.17 mol), MIBK (200 g), and 3,5-lutidine (30.1 g, 0.28 mol). The contents were stirred for 10 minutes. To the reaction mixture 2,2,2-trifluoroacetyl chloride (TFAC) (36.4 g, 0.28 mol) was added whilst maintaining the temperature at 25° C. After 2 hours, water (100 mL) was introduced and the contents were stirred for 5 minutes, the layers were separated, and the organic layer was washed with water (100 mL) and aq. NaHCO₃ (2×100 mL). The organic layer was concentrated under reduced pressure to afford 61 g (94% yield, 95% purity) of compound (I-B) as oil. Occasionally the oil solidified upon storage, in an uncontrolled manner.

¹H NMR (400 MHz, CDCl₃) δ ppm: 8.09 (d, 2H), 7.48 (d, 2H), 4.98 (m, 1H), 4.80 (m, 1H), 4.27 (m, 1H), 3.71 (s, 3H), 3.34 (s, 3H), 1.37 (d, 3H). ¹⁹F NMR (400 MHz, CDCl₃) δ ppm: −65.35 (s).

### 2b) Preparation of Crystal Seeds of Compound of Formula (I-B)

The oil as obtained under 2a) was dissolved in methylcyclohexane, the resulting mixture heated to 70° C. until complete solution of the oil and the resulting solution was cooled slowly to room temperature and held at this temperature until crystal formation was visible. The solution was further cooled to −5° C. The obtained crystals were filtered off and washed with methylcyclohexane to obtain compound (I-B) as a white crystalline product.

### 2c) Preparation of Polymorph of Compound (I-B)

The oil was dissolved in methylcyclohexane at 70° C., the solution was cooled to 40° C. and seed crystals were added. The solution was further cooled to −5° C. and stirred for 1 hour. The obtained crystals were filtered off and washed with methylcyclohexane to obtain a white crystalline product (56.0 g, 97.6% purity, 88% yield). Single-crystal X-ray diffraction analysis confirmed the presence of the polymorph of compound (I-B) of the invention.

### Analysis of Polymorph

After preparation by the methods detailed above, the samples were subject to analysis by powder X-ray diffraction and/or single crystal X-ray diffraction and/or differential scanning calorimetry (DSC).

Powder X-Ray Diffraction Analysis (pXRD):

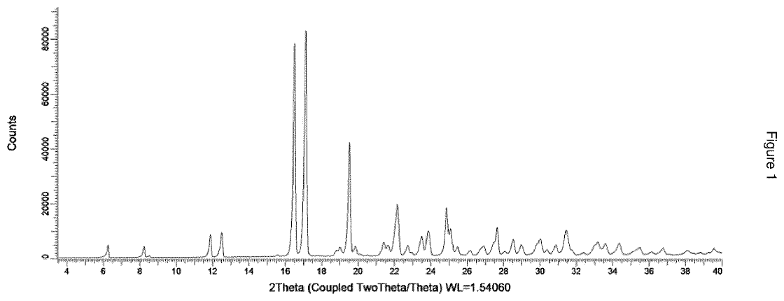

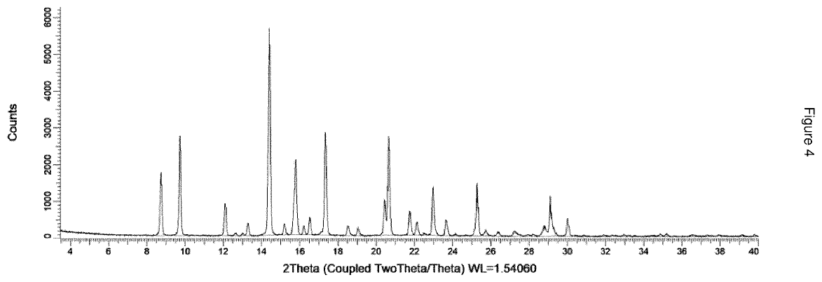

Powder X-ray diffraction analysis of solid material was carried out using the Bruker D8 (pXRD) powder diffractometer with Lynxeye detector at room temperature and at relative humidities above 40%. The samples were packed into the XRD holder and the surface of the sample levelled with a microscope slide. The pXRD holder was placed in the instrument, spun and the powder pattern collected from 3.5° to 40° 2-theta, with a scan time of 25 to 30 minutes depending on the pattern intensity. Measured powder X-ray diffraction patterns for the polymorph of compound (I-A) and compound (I-B) A are shown in FIG. 1 and FIG. 4, respectively.

### Single Crystal Intensity Data:

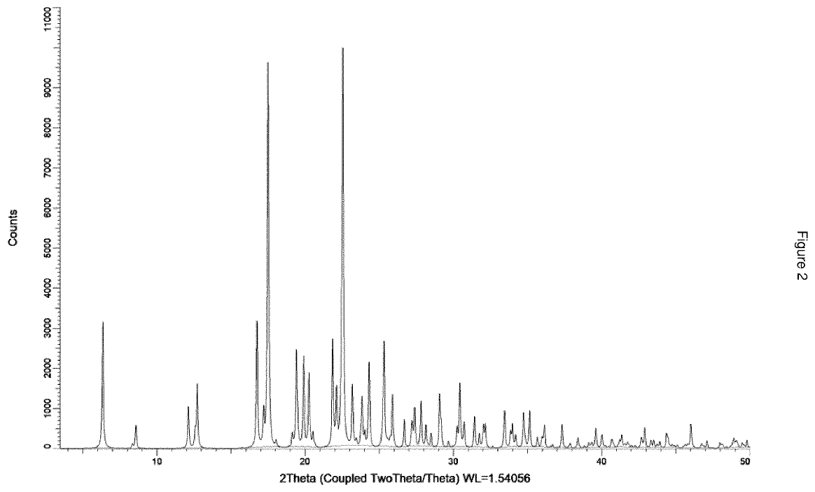

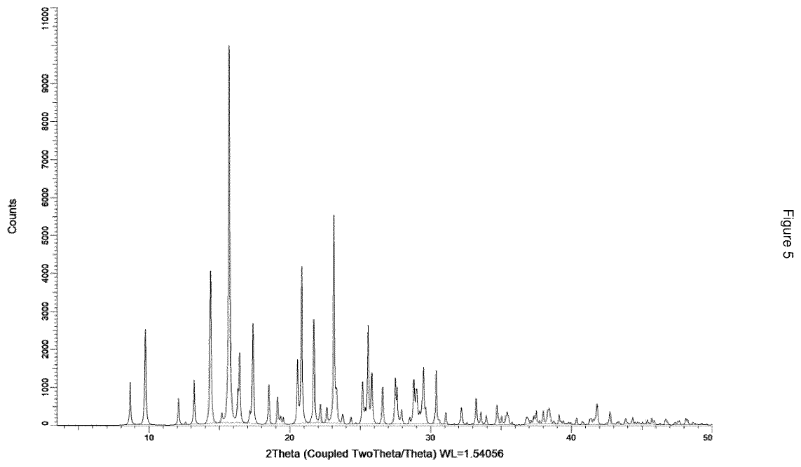

Single crystal intensity data was collected on an Rigaku SuperNova diffractometer using Cu Kα radiation (λ=1.54056 Å) with a graphite monochromator. The crystal was mounted in NVH oil at 100 K for data collection. The data was solved using the CRYSTALS software package. This data was used to produce a predicted powder X-ray diffraction pattern for the polymorph of compound (I-A) (FIG. 2) and compound (I-B) (FIG. 5).

### Differential Scanning Calorimetry (DSC):

DSC was carried out using a Mettler Toledo DSC1. A sample loading of around 1-3 mg was loaded in a 40 μL aluminum sample holder and heated from 25° C. to 260° C. at a rate of 10° C./minute. The lid of the DSC crucible was pierced to allow the escape of any gas formed during the heating of the sample.

The DSC analysis confirmed the presence of the polymorphs of the invention with a melting point of compound (I-A) and compound (I-B):

| | | |
|---|---|---|
| Compound | Sample loading | Melting point |

| | | | |
|---|---|---|---|
| I-A | 2.57 | mg | 38.6° C. |
| I-B | 1.8 | mg | 63.5° C. |

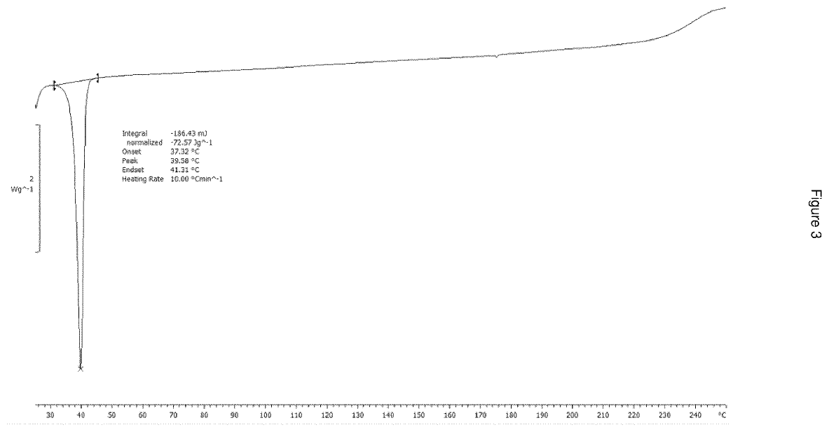

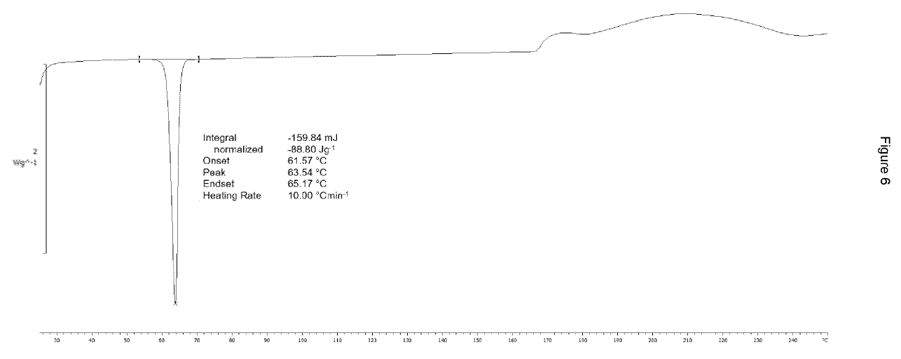

The DSC trace for the polymorph of compound (I-A) is shown in FIG. 3 and for the polymorph of compound (I-B) A in FIG. 6, respectively.

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims. All publications cited herein are hereby incorporated by reference in their entirety for all purposes to the same extent as if each individual publication were specifically and individually indicated to be so incorporated by reference

1. A crystalline polymorph of the compound of formula I-A 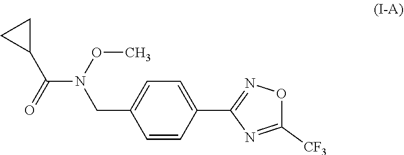 [SMILES: CON(Cc1ccc(-c2noc(C(F)(F)F)n2)cc1)C(=O)C1CC1]
which has an X-ray diffraction pattern comprising four or more 2-theta angle values selected from the group 11.9±0.2, 12.5±0.2, 16.5±0.2, 17.1±0.2, 19.5±0.2, 22.1±0.2, 23.9±0.2 and 24.9±0.2.

2. A crystalline polymorph of the compound of formula I-B 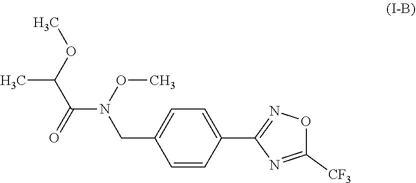 [SMILES: COC(C)C(=O)N(Cc1ccc(-c2noc(C(F)(F)F)n2)cc1)OC]
which has by an X-ray diffraction pattern comprising four or more 2-theta angle values selected from the group 8.7±0.2, 9.7±0.2, 14.4±0.2, 15.8±0.2, 17.3±0.2 and 20.7±0.2.

3. The crystalline polymorph of formula (I-A) of claim 1, which has the following lattice parameters: a=15.2±0.1 Å, b=4.8±0.1 Å, c=21.5±0.1 Å, α=90±0.2º, β=106.4±0.2º, γ=90±0.2º and volume 1508±16 Å³.

4. The crystalline polymorph of formula (I-A) of claim 1, which has a melting point of 38.6° C.

5. The crystalline polymorph of formula (I-B) of claim 2, which has the following lattice parameters: a=7.4±0.1 Å, b=18.2±0.1 Å, c=12.7±0.1 Å, α=90±0.2º, β=104.3±0.2º, γ=90±0.2º and volume 1664±17 Å³.

6. The crystalline polymorph of formula (I-B) of claim 2, which has a melting point of 63.5° C.

7. An agricultural composition comprising a polymorph of formula (I-A), or polymorph of formula (I-B) as claimed in claim 1 and at least one agriculturally acceptable carrier or diluent.

8. A method of controlling or preventing infestation of useful plants by phytopathogenic microorganisms, wherein a fungicidally effective amount of a polymorph of formula (I-A) or polymorph of formula (I-B) according to claim 1, or a composition comprising said polymorph as active ingredient, is applied to the plants, to parts thereof or the locus thereof.

9. The method according to claim 8, wherein the compound is the compound of formula I-A.

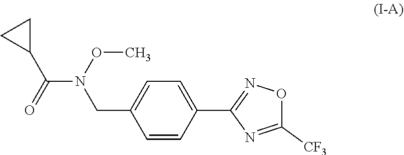 [SMILES: CON(Cc1ccc(-c2noc(C(F)(F)F)n2)cc1)C(=O)C1CC1]

10. The method according to claim 8, wherein the compound is the compound of formula I-B.

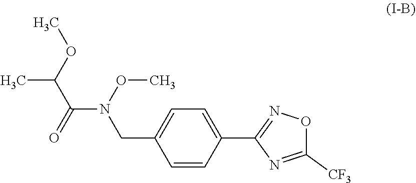 [SMILES: COC(C)C(=O)N(Cc1ccc(-c2noc(C(F)(F)F)n2)cc1)OC]

11. The method according to claim 8, wherein the plants are genetically modified plants selected from soybean.

12. The method according to claim 8, wherein said plants are Bt soybean plants.

13. Use of a polymorph of formula (I-A) or a polymorph of formula (I-B) according to claim 1 as a fungicide.

14. Use of a polymorph of formula (I-A) or a polymorph of formula (I-B) according to claim 1 for controlling Phakopsora pachyrhizi in genetically modified soybean plants.

15. The use according to claim 14, wherein said genetically modified soybean plants are Bt soybean plants.
